(19) United States
(12) Patent Application Publication
(10) Pub. No.: US 2026/0250630 A1
(43) Pub. Date: Aug. 27, 2026

(54) PROTEOMICS-BASED RECEPTOR-LIGAND MATCHING FOR OPTIMIZING STEM CELL REPROGRAMMING

(71) Applicant: OKINAWA INSTITUTE OF SCIENCE AND TECHNOLOGY SCHOOL CORPORATION, Okinawa (JP)

(72) Inventors: Zacharie TAOUFIQ, Okinawa (JP); Dimitar DIMITROV, Okinawa (JP); Marina KHANDARKHAEVA, Okinawa (JP); Tomoyuki TAKAHASHI, Okinawa (JP); Alejandro VILLAR-BRIONES, Okinawa (JP); Roy C. MICHEAL, Okinawa (JP)

(21) Appl. No.: 18/715,004

(22) PCT Filed: Nov. 30, 2022
(86) PCT No.: PCT/JP2022/044253

§ 371 Date: May 30, 2024

(30) Foreign Application Priority Data
Nov. 30, 2021 (JP) 2021-194719

(51) Int. Cl. C12N 5/0793; A61K 35/30

(52) U.S. Cl. CPC C12N 5/0619; A61K 35/30

(57) ABSTRACT

A composition comprising a ligand for modulating a function of a stem cell-derived cell culture is provided, along with method for identifying a protein in a population of proteins in a tissue and/or an organ and/or an in vitro cell culture and/or purified subcellular fraction is also provided. A method is also provided for quantifying a target protein in a tissue and/or an organ and/or an in vitro cell culture and/or purified subcellular fraction, further comprising identifying a protein, the expression level of which is larger or lower in the iPSC- or ESC-derived cell culture than in the iPSC or ESC culture, and identifying a ligand or transcription factor against the protein, the expression level of which is larger or lower in the iPSC- or ESC-derived cell culture than in the iPSC or ESC culture.

### CROSS-REFERENCE TO RELATED APPLICATIONS

This application is a national stage filing under 35 USC 371 of International Application No. PCT/JP2022/044253, filed Nov. 30, 2022, and claims the benefit of Japanese Application No. 2021-194719, filed Nov. 30, 2021, each application of which is hereby incorporated herein by reference in its entirety.

### SEQUENCE LISTINGS

The instant application contains a sequence listing which has been submitted in XML format via EFS-Web in the parent International Application No. PCT/JP2022/044253, filed Nov. 30, 2022, which is hereby incorporated by reference in its entirety. Said XML copy, created on May 29, 2024, is named JPOXMLDOC01-seql.xml and is 34,602 bytes in size.

### BACKGROUND

### Field of the Invention

The present invention relates to a method for identifying a hidden protein, a method for identifying a ligand against the protein, a method for quantifying a target protein, a protein or a ligand identified by the method, and uses thereof.

The present invention also relates to a method for identifying a proteotypic peptide, a method for producing an isotope-labeled proteotypic peptide, a peptide or an isotope-labeled peptide produced by the method, and a composition comprising the peptide or the isotope-labeled peptide. The present invention also relates to a composition comprising a ligand for modulating a function of a stem cell-derived cell, for optimizing stem cell differentiation in the stem cell-derived cell culture, wherein the ligand is determined based on ‘ultra-definition’ (UD) proteomics.

The present invention also relates to a method for diagnosing a disease and/or a disorder in a subject, a method for disease modeling, disease therapy and immunotherapy, a method for treating a disease and/or a disorder associated with a nervous system in a subject, comprising administering an effective amount of the ligand.

### Background Art

In the non-targeted proteomics, which was conventionally used, protein samples are fractionated by liquid chromatography, and m/z spectra and peptide sequences are obtained by tandem mass spectrometry (MS/MS). However, this method has a problem that the number of detectable proteins is limited. Particularly in the case of a complex sample such as a biological sample, even if it is separated by liquid chromatography, each fraction contains many peptides with similar mass but different sequences. Then, from the spectra derived from the various peptides observed in the MS of the first step, one having a high spectral intensity is automatically selected as a parent ion and fragmented, and the m/z spectrum of the generated fragment ion is obtained. Then, from the spectrum, the sequence of the peptide from which the parent ion originates is determined. As described above, since this method analyzes preferentially those with high spectral intensity, proteins with high abundance are analyzed preferentially, whereas proteins with low abundance are not analyzed, or it will take a very long time to analyze proteins with low abundance. In addition, in the case of two or more peptides with similar mass but different sequences, only the most abundant ones are automatically analyzed.

On the other hand, there is targeted proteomics as a method for solving the above problem. In this method, parameters such as retention time of liquid chromatography are set so that it can be analyzed by focusing only on specific peptides derived from the protein to be analyzed. Moreover, a peptide labeled with a stable isotope having the same sequence as the target peptide (hereinafter referred to as “stable isotope proteotypic peptide”) was mixed in a sample in a known amount and analyzed, and it becomes possible to estimate the abundance of the target protein in the sample by comparing the area ratio of the m/z spectrum (of parent ions before fragmentation) of the native peptide and the stable isotope proteotypic peptide.

However, with this method, it is necessary to select proteotypic peptide from the sequence of the protein to be analyzed that is ‘visible’ or detectable by mass spectrometry. As to proteotypic peptide, if there is a peptide sequence published in a paper or the like so far, such peptide sequence may be used, but in the absence of such a known peptide sequence, a peptide sequence selected by software shall be used. However, the number of peptide sequences that can be proteotypic peptides published in papers etc. is still small, and the candidate sequences of proteotypic peptides selected by software are often not detectable even when analyzed by mass spectrometry in practice, it is necessary to enrich libraries of reliable proteotypic peptides.

The hidden proteome cannot be confirmed and monitored with antibody-based technique. Problems of antibodies for exploring unknown proteins are as follows:
costly and time-consuming to make for an unknown protein (e.g. express and purify the target protein, immunize animals over several months, serum collection, ab purification and testing);
availability, for a large majority of the proteome antibodies are still not commercially available;
detectability, may not detect lower abundant protein ranges;
accessibility, may not bind the target within complexes; and
specificity, may not recognized isoforms or closely related proteins within families.

The functions of eukaryotic cells, in all their complexity, depend upon highly specific compartmentalization into subcellular domains, including organelles. These compartments represent functional units characterized by specific supramolecular protein complexes. A major goal of modern biology is to establish an exhaustive, quantitative inventory of the protein components of each intracellular compartment. Such inventories are points of departure, not only for functional understanding and reconstruction of biological systems, but also for a multitude of investigations, such as evolutionary diversification and derivation of general principles of biological regulation and homeostasis.

Essential to communication within the nervous system, chemical synapses constitute highly specific compartments that are connected by axons to frequently distant neuronal cell bodies. Common to all chemical synapses are protein machineries that orchestrate exocytosis of synaptic vesicles (SVs) filled with neurotransmitters in response to presynaptic action potentials, resulting in activation of postsynaptic receptors. Moreover, synapses are composed of structurally and functionally distinct sub-compartments, such as free and docked SVs, endosomes, active zones (AZs) at the presynaptic side, and receptor-containing membranes with associated scaffold proteins on the postsynaptic side. Thus, it is not surprising, that mass-spectrometry (MS)-based proteomics, combined with subcellular fractionation, yields protein inventories of high complexity. For instance, >2,000 protein species were identified in synaptosomes (1), ~400 in the SV fraction (2), ~1,500 in post-synaptic densities (3) and ~100 in an active zone (AZ)-enriched preparation (4).

While these studies provide insights into the protein composition of synaptic structures, they are still inherently limited for two reasons. First, synapses are functionally diverse with respect to the chemical nature of their neurotransmitters, as well as their synaptic strength, kinetics, and plasticity properties (5). Therefore, analyzed subcellular fractions represent ‘averages’ of a great diversity of synapses (6) or SVs (2). The second limitation is that proteins known to be present in specific subsets were not found in these studies despite the unprecedented sensitivity of modern mass spectrometers. In fact, many functionally critical synaptic proteins have remained undetected. For example, the synaptotagmin (Syt) family, major Ca²⁺ sensors of SV exocytosis, comprises >15 members, of which only 5 had been identified in previous SV proteomics (2, 4, 7). Missing isoforms included Syt7, involved in asynchronous transmitter release (8), synaptic plasticity (9), and SV recycling (10). Likewise, the vesicular transporters for monoamines (VMATs) and acetylcholine (VAChT) neurotransmitters were missing in these studies. Clearly, known components of the diversified synaptic proteome have been missing, and it is not possible to predict how many more such proteins remain hidden.

What are the reasons for the continuing incompleteness of synaptic protein inventory? Proteome identification and quantification rely heavily on MS detectability of peptides generated by digestion of extracted proteins with sequence-specific enzymes, such as trypsin. However, in MS analysis of complex biological samples, peptide signals from a few abundant proteins often mask those that are less abundant. Additionally, the probability of obtaining peptides with similar masses, but different amino acid sequences, increases with increasing sample complexity (11, 12). To overcome these limitations, we have elaborated a workflow with dual-enzymatic protein digestion in sequence combined with an extensive peptide separation prior to MS analysis. As proof-of-concept, we have utilized purified SV fractions from rat whole brain, which serve as a benchmark for quantitative organellar proteomics (2). As a result, we detected ~1,500 proteins in the SV fraction, three times more than reported previously. This new proteome not only covers all known canonical SV proteins, but also contains proteins previously overlooked such as the low-abundance Syts and SV transporters. Moreover, peptide quantification allowed for differentiating ‘SV-resident’ from ‘SV-visitor’ proteins. In fact, most ‘SV-resident’ proteins newly detected in our SV proteomics are of low abundance, with an average copy number of less than 1 per SV, suggesting a larger molecular and functional diversity of SVs than previously thought. Remarkably, more than 200 proteins detected in the SV fraction are genetically associated with brain disorders, 76% of which are newly identified in the SV fraction.

Eukaryotic cells organize their intracellular space into multiple specialized membrane bounded and unbounded cytosolic compartments. These compartments contain a high density of organized proteins machineries that collectively control and perform almost all biological reactions. Subcellular compartmentalization is a fundamental life strategy which has allowed cells to optimize activities and interactions of their proteins, creating many new biological processes. In multicellular organisms, specialized subcellular compartments exist in many copies. These compartments comprise stable core proteome, but at the same time, a part of their proteome has undergone great diversification in time and space (Jacob, 2001; Holland, 2009). This complex molecular diversity is foundational to the evolution and emergence of new and more sophisticated biological processes. Therefore, to resolve the mechanisms of complex life phenotypes, it is essential to characterize the deep organization of subcellular proteomes and monitor their spatio-temporal dynamics.

In the brain, synapses constitute a striking example of a subcellular compartment of high physiological importance. Synapses not only connect neuronal cells to one another, but also play a central role in the process/storage/control of information that flows within neural circuits. Common to all chemical synapses are protein machineries that orchestrate membrane fusion of neurotransmitter-containing vesicles following presynaptic action potentials and activation of receptors at the postsynaptic side by the released neurotransmitter. However, beyond intrinsic transmission process and the canonical proteome, synapses are functionally diverse and may operate on a wide range of synaptic transmission strength, kinetics and plasticity properties (Abbott and Regehr, 2004; O'Rourke et al., 2012). Thus, anatomical and functional specializations of brain neural circuits are thought to arise from molecular diversity in different types of synapses. The deep diversity of the synaptic proteome may underlie cognition capacities, learning, memory processes, and other complex attributes of mammalian brain (Emes and Grant, 2012). In fact, mutations of genes encoding synaptic proteins frequently accompany human mental and neurological disorders (Grant, 2012).

Mass-spectrometry (MS)-based proteomics, combined with subcellular fractionation, has provided an extensive inventory of proteins species detected in synaptic fractions with >2000 species in synaptosomes (Biesemann et al., 2014), ~400 in synaptic vesicles (SVs) (Takamori et al., 2006), ~1500 in the post-synaptic density (Bayes et al., 2012), and ~100 in the active zone (Boyken et al., 2013)). However, current quantitative proteomes for synaptosomal (Wilhelm et al., 2014) and SV proteins (Takamori et al., 2006) are restricted mostly to ubiquitous and abundant proteins, missing number of functionally characterized. Also, in these synapse proteomics inventories, many disparately expressed in the brain, yet functionally critical proteins are still missing. For example, the synaptotagmin (Syt) family (major Ca²⁺ sensors of SV exocytosis in neurons) comprises at least 15 distinct members beyond the canonical Syt1 and Syt2 (Sudhof, 2002; Chen and Jonas, 2017). However, present SV proteomics typically identify up to 5 of them (Burre et al., 2006; Takamori et al., 2006; Gronborg et al., 2010; Boyken et al., 2013). Missing isoforms include Syt7, yet recently highlighted in short-term synaptic plasticity (Jackman et al., 2016; Turecek et al., 2017), asynchronous transmitter release (Li et al., 2017) and/or SV recycling (Liu et al., 2014; Chen et al., 2017). Other prominent missing examples are among transporters that fill vesicles with non-ubiquitous neurotransmitters (NT). Beyond the canonical (~90% of synapses) NTs glutamate (excitatory synapses) and GABA (inhibitory synapses), the brain uses various less ubiquitous yet physiologically essential NTs such as dopamine, serotonin, histamine, and norepinephrine loaded into SVs by vesicular monoamine transporters (vMAT1 and 2). Likewise, vesicular acetylcholine transporter (vAChT), which is used for SV filling with ACh, remains undetected in SV proteomics. Therefore, at present, it is impossible to determine how much of the diversified synaptic proteomes remain hidden. (v) safe, stable and easily available and administrable.

Proteome identification and quantification rely heavily on the MS detectability of specific peptides generated by digestion of extracted proteins with a sequence-specific enzyme, such as trypsin. However, in complex biological mixtures, the peptide signals from few abundant proteins often mask lots of those from less abundant ones. Additionally, the probability of getting peptides with similar masses but different amino acid sequences remain high (Righetti and Boschetti, 2007; Aebersold and Mann, 2016). Thus, many functionally critical synaptic proteins remain undetected, being masked by more abundant and/or structurally similar proteins, even using a high-resolution MS device.

Here we elaborated a multi-enzymatic protein digestion method with a peptide separation based on the multiple biophysical properties of amino acids, searching for peptide signatures of hidden synaptic proteins in the highly purified SV fraction from rat whole brain tissue homogenates (Takamori et al., 2006). As a result, we uncovered ~1,500 proteins in the SV fraction. a proteome three times larger than previously reported. This new proteome includes all the canonical SV proteins, but also the less ubiquitous synaptotagmins and NT transporters found in restricted SV populations in the brain. Moreover, our approach quantitatively revealed the organization of the SV proteome within synapses, with two spatially distinct repertoires: proteins residing with SVs and those transiently interacting with SVs. Most SV proteins newly detected in our proteomics had a copy number less than 1 per SV, revealing a larger SV molecular and functional diversity than previously thought. We constructed a database listing nomenclature, structural, functional and pathological information related to all the detected SV fraction proteins. We demonstrated that this resource can be utilized for exploring unidentified compositions and organizations of the SV proteome, such as SV kinome, rabome, vacuolar (v) ATPase complex, SV transporters and novel SV-resident proteins. More importantly in our data analyses, we found 236 brain disorders associated with 210 proteins detected in the SV fraction, of which 159 proteins (76%) were hidden to previous proteomics approach. Thus, our study has reached a deep subcellular proteomics, by uncovering a large and previously hidden SV proteome of functional importance and with a complex and diverse spectrum. Our new method marks a significant milestone towards the deep explorations of (subcellular) synaptic proteomes and enables their dynamics (variome/variation) during development, evolution and/or diseases of the brain.

### SUMMARY OF THE INVENTION

Synapses are specialized cellular structures connecting neurons that are essential to communication within the brain. They receive-process-store-control all information that flows within neuronal networks. In fact, alteration of synaptic protein expression is often at the root of many brain diseases such as Alzheimer's, autism, ADHD, and schizophrenia. Therefore, there is a tremendous interest in dissecting their whole protein composition or ‘proteome’.

As current knowledge of the full proteome of mammalian synapses was still lacking, we have recently developed a new synapse proteomic workflow (‘UD proteomics’) using animal models (Taoufiq et al, PNAS 2020, Taoufiq et al.). This protocol enables the identification and quantification of 4,500 synaptic proteins species (including 1,500 synaptic vesicles proteins), three times more than any reported previously (Takamori S et al, Cell 2006; Wilhelm B G et al, Science 2014; Koopmans et al, Neuron 2019). This is because the workflow, compared to others, uses extensive protein digestion steps and a synapse-specific multi-dimensional peptide separation during proteomic sample preparation, resolving in particular the many overlapping peptide signals in mass spectrometry with same masses but different amino sequences. Conditions of the UD workflow (number of fractions, solvents pH, ERLIC gradients, chromatography duration, etc.) were optimized for our synaptic samples.

More importantly, our data have revealed more than 200 synaptic vesicles proteins genetically associated with distinct brain disorders, 76% of which were undetectable by current published studies.

The low abundant synaptic proteome is strongly related to neurological disorders: (A) Numbers of synaptic vesicles (SV) proteins identified in Takamori et al Cell 2006 and using the synapse UD proteomics workflow. (B) Quantitative representation in a rank-abundance plot of the SV proteome. Y-axis: log base 10 of iBAQ score; X-axis: abundance score rank in the proteome. Proteins having disease(s) caused by mutation(s) affecting the gene represented in the database entry are indicated (black circles). Of the ~1,500 reported SV proteins, 210 are genetically associated with distinct brain diseases. Remarkably, a majority of these are low abundant and were ‘invisible’ to previous synapse proteomics studies (Takamori et al Cell 2006; Wilhelm et al Science 2014; Koopmans et al Neuron 2019)

We have optimized iPS reprogramming and cell culture medium by means of our proteomics workflow (Taoufiq et al PNAS 2020) to make healthier and more ‘connected’ differentiated neurons. UD proteomics allows to detect and quantify receptors that are not seen by conventional proteomics. We added matching ligands in the medium, thereby neuronal growth and synaptogenesis are largely improved. Neurons are more functional.

IPS cell reprogramming is a relatively new field (<10 yrs) with great potential in applications such as regenerative medicine and drug discovery. Reprogramming methods were made to simply differentiate cells into basic neurons. However, little has been done on optimizing neuronal morphogenesis and functions. For example, one conventional method is to supplement cell culture medium with BDNF and NT3 growth factors. With our proteomics data, we showed that BDNF receptors TrKB but not NT3 receptors TrKC is expressed in the reprogrammed neurons. Thus, NT3 could be excluded from the medium. Additionally, proteomics data revealed the presence of CNTFR, GDNFR1, GDNFR2, GDNFR3, FGFR2, FGFR3 receptors on reprogrammed neurons. Thus, we match with their ligands (CNTF, GDNF, FGF16, FGF22) by adding those in the medium. As a result, this significantly improved neuronal morphogenesis and enhanced synapse formation. This process may be adapted for further stem cell reprogramming into any cell types.

Current proteomics studies clarified canonical synaptic proteins that are common to many types of synapses. However, proteins of diversified functions in a subset of synapses are largely hidden because of their low abundance or structural similarities to abundant proteins. To overcome this limitation, we have developed an ‘ultra-definition’ (UD) subcellular proteomic workflow. Using purified synaptic vesicle (SV) fraction from rat brain, we identified 1,466 proteins, three times more than reported previously. This refined proteome includes all canonical SV proteins as well as numerous proteins of low abundance, many of which are newly identified. Comparison of UD quantifications between SV and synaptosomal fractions has enabled us to distinguish SV-resident proteins from potential SV-visitor proteins. We found 134 SV-residents, of which 86 are present in average copy number per SV less than one, including vesicular transporters of non-ubiquitous neurotransmitters in the brain. We provide a fully annotated resource of all newly categorized SV-residents and potential SV-visitor proteins, which can be utilized to drive novel functional studies, as we characterized here Aak1 as a novel regulator of synaptic transmission. Moreover, proteins in SV fraction are associated with more than 200 distinct brain diseases. Remarkably, a majority of these proteins was found in the low-abundance proteome range, highlighting its pathological significance. Our deep SV proteome will provide a fundamental resource for a variety of future investigations on the function of synapses in health and disease.

The keywords relating to the present invention are proteomics, iPS cell differentiation, neurons, growth factors, synaptogenesis, receptors, ligands, synapse, deep proteomics, synaptic vesicles, brain disorders, neurotransmission, hidden proteome, peptide synthesis, neurological diseases, mass spectrometry identification and quantification.

Stem cell differentiation methods were made to simply differentiate iPS cells into other cell types. However, little has been done on optimizing the differentiated cells' morphogenesis and functions. Yet, this may be crucial in the growing field of regenerative medicine. Based on our UD proteomics method, we are able to create new cell culture recipes and viral-based differentiation paths to largely improve the health and functions of iPSC-derived cells. we have optimized differentiation to make psychiatric patients' iPSC-neurons healthier and ‘more connected’. UD-proteomics allowed to detect and quantify numerous receptors and nuclear factors that remain invisible to proteomics. We added selected matching ligands in the medium, thereby neuronal growth, synaptogenesis and activity were largely improved. This process may be adapted for further differentiation of iPSCs into any cell types, such as iPSC-derived heart, skin, liver, lung, retinal, and pancreatic cells.

Mammalian central synapses of diverse functions contribute to highly complex brain organization, but the molecular basis of synaptic diversity remains open. This is because current synapse proteomics are restricted to “average” composition of abundant synaptic proteins. Here we demonstrate a subcellular proteomic workflow that can identify and quantify the deep proteome of synaptic vesicles, including previously missing proteins present in a small percentage of central synapses. This synaptic vesicle proteome newly detected many proteins of physiological and pathological relevance particularly in low abundance range, thus providing a resource for future investigations on diversified synaptic functions and neuronal dysfunctions.

According to the present invention disclosure, conditions of proteolysis were optimized (conditions of denaturation, conditions of proteolytic enzyme digestion), then fractions were fractionated by a column called ERLIC, and peptides contained in each fraction were analyzed by LC MS/MS (hereinafter referred to as UD method). This method per se is an optimization of conditions published so far in papers and the like for proteins of the synapse fraction. However, as a result of this condition examination, proteins which were known to be in synapses but have not been detected by mass spectrometry, or proteins which were annotated as genes but whose roles and expression sites were not known were detected, and the type of protein detected by mass spectrometry increased more than three times from the time of 2006 (from 408 types to 1500 types).

We focused on one protein SVx from the proteins thus newly detected in the synaptic vesicle fraction, and analyzed it. A stable isotope proteotypic peptide in which lysine at the C-terminus (peptides after proteolysis with trypsin and lysC become those with lysine or arginine at the C terminus) was synthesized with lysine containing the stable isotopes 13C and 15N with respect to a peptide sequence in SVx detected in the UD method was mixed in a sample at a known concentration, and the UD method was performed to estimate the concentration in the sample by comparing the signal intensity of the native peptide sequence with the signal intensity of the stable isotope proteotypic peptide.

By quantitatively analyzing the protein found in synaptic vesicles in this manner, we succeeded in estimating the number of molecules per synaptic vesicle. As a result, it became clear that 236 kinds of cranial nerve-related diseases are associated with 210 synaptic vesicle proteins and that many proteins with a small number of molecules per synaptic vesicle are involved in brain-related diseases. We believe that, from now on, by using the proteotypic peptides obtained in this analysis and quantitatively analyzing synapse-related proteins in fractions other than synaptic vesicles by the UD method, it is possible to clarify the localization and dynamics at the time of normal in the synapse, and by further studying those changes in brain-related diseases, it will be useful for elucidation and diagnosis of the mechanism of brain diseases in the future.

The human brain is capable of complex intelligence because it is composed of billions of neurons assembled into communicating high-order networks, connected by trillions of specialized subcellular structures called ‘synapses’. Synapses not only connect neuronal cells, but also receive/process/store/control information that flows within neural circuits. Therefore, there is tremendous interest in dissecting their specific proteome.

In the synapse, proteins machineries orchestrate exocytosis of neurotransmitter-containing synaptic vesicles (SV) in response to presynaptic action potentials, which is followed by activation of receptors at the postsynaptic side.

Thus, mutations and abnormalities of many synaptic proteins are related to brain diseases. However, it is hardly understood how each synaptic protein changes in each disease condition. In the future, in order to understand molecular mechanisms underlying individual brain diseases and develop patient-personalized therapeutic methods for each disease, it is essential to understand the changes of various synaptic proteins for each patient disease.

In order to comprehensively and quantitatively understand synaptic protein changes in brain diseases, the UD method which can comprehensively analyze almost all synaptic proteins is effective. The UD method comprises the steps of (i) purification of synapse fraction, (ii) 2-step proteolysis with lysC and trypsin, (iii) fractionation of peptide by ERLIC column, and (iv) RPC LC-MS/MS. In Takamori et al., 2006, the step (ii) uses trypsin only, and the step (iii) does not exist.

In order to analyze changes in synaptic proteins in brain diseases, previously, antibodies against synaptic proteins to be studied have been prepared; disease condition and normal condition have been compared using Western blotting, antibody staining, etc., with the antibodies; changes in synaptic proteins to be studied in the disease condition have been analyzed. However, with this method, it was no exaggeration to say that it is impossible to capture the comprehensive change of synaptic proteins in disease conditions due to limitations of the time and cost of antibody production and the number of samples that can be processed by one analysis. On the other hand, in proteome analysis using mass spectrometry, the types of proteins that can be analyzed at one time are far greater than those using antibodies, and it is possible to quantitatively analyze these proteins. However, there is nevertheless a problem that the types of proteins that can be detected in the synapse fraction are fewer than those present in synapses, and even proteins that have been shown to exist in synapses by analytical methods using antibodies cannot be detected from the synapse fraction in mass spectrometry.

We succeeded in dramatically improving the detection sensitivity of mass spectrometry by the UD method and making it possible to detect almost all of synaptic proteins from synapse fraction. Such improvement of the detection sensitivity is a result of optimizing each step of the UD method and it is not easily obtained. In the future, by analyzing human neurons induced from patient cell-derived iPS and disease animal model by the UD method, it is possible to capture the changes (increase and decrease, change in distribution, post-translational modifications) of each synaptic molecule comprehensively, and it is expected to be able to understand disease conditions more deeply.

The peptide detected by the UD method can be used as a proteotypic peptide for synaptic protein in the future (see JP-A 2010-085103 “peptide used for simultaneous protein quantification of metabolic enzyme group using mass spectrometer” because it is similar in idea). For example, in order to know the quantitative change of a specific synaptic protein B in disease model mouse A, a known amount of a proteotypic peptide of synaptic protein B is added to each of synapse fraction derived from disease model mouse A and synaptic fraction derived from normal mouse and analysis with mass spectrometry is made. The concentration of protein B in the synaptic fraction of the disease model mouse A and normal mouse can be determined and compared by comparing the signal intensity of the target peptide and the signal intensity of the added proteotypic peptide.

Although the sequence of the synapse protein B itself is known, it has been unclear in many cases (especially in the case of small amount of protein present in the synapse or proteins present in a subset of synapses) which part of peptide therein can be detected by mass spectrometry and how easily it can be detected even if it can be detected. Thus, in case that a proteotypic peptide is not known when quantitatively analyzing synaptic proteins by mass spectrometry, there is no options except for using peptides selected from computer-presented lists as proteotypic peptides, but the probability for those actually able to be detected was very low. Thus, if there is information on peptides that can actually be detected by mass spectrometry, it is possible to reduce the enormous cost that has been involved in the synthesis of peptides that are not known whether can be used or not. Thus, based on the list of peptides of synaptic proteins that can be detected using the UD method at this time, we are planning to chemically synthesize proteotypic peptides in which lysine or arginine at the C terminus is labeled with stable isotope (peptides after proteolysis with lysC and trypsin become those with lysine or arginine at the C terminus) and commercialize them for research and diagnosis.

In the disclosed studies, a list of proteotypic peptides of rat synaptic proteins has been obtained. Many synaptic proteins are conserved in mammals, but by selecting and using peptides conserved among humans and mice, they can be used for analysis in those species.

With the list of peptides, we can design and synthesized the correct peptide for identification and absolute quantification of any synaptic proteins. The peptides are superior to antibodies for both detection and quantification. The idea is to build up a company which will sell peptides for the study of synaptic proteins, based on the UD list, which contain about 110,000 peptides to protect.

Synaptic dysfunction is a major determinant of neurological diseases. With current proteomics approaches, a large part of the synaptic proteome has remained hidden. We have developed a new approach called ‘Ultra-definition proteomics’ for deep subcellular proteome identification and quantification. As a Proof-of-Concept: we have tripled the known proteome of synaptic vesicles (SV) previously described in Takamori et al Cell 2006 (from 409 to 1483 proteins). Many novel SV accessory protein groups, isoforms and lower abundant proteins were revealed and quantified. One aspect of the hidden SV proteome analysis was particularly unexpected: 236 distinct brain diseases were found associated with 210 SV-interacting proteins, of which 159 (=76%) were non-reported proteins in Takamori et al Cell 2006, bringing to light the immense challenge we are facing for drug targeting of neurological disorders in the 21st century. The ‘UD peptide list or library’ generated by the new method is a key knowledge to further track, confirm and quantify the presence of any synaptic protein in complex samples using a highly specific and precise peptide-based technology.

### In Some Embodiments, the Present Invention Relates to the Following:

<1> A composition comprising a ligand for modulating a function of a stem cell-derived cell.
<2> The composition of <1>, wherein the ligand is one or more selected from the group consisting of FGF3, FGF7, FGF10, FGF22, FGF8, FGF9, FGF16, FGF17, FGF18, FGF20, BDNF, GDNF, NRTN, PSPN, and CNTF.
<3> The composition of <1> or <2>, wherein the ligand is FGF22 or a combination of (i) BDNF and GDNF, (ii) BDNF, CNTF, and GDNF, (iii) BDNF, CNTF, GDNF, and FGF16, or (iv) BDNF, CNTF, GDNF, FGF16, and FGF22.
<4> The composition of any one of <1> to <3>, wherein the stem cell-derived cell is selected from the group consisting of a stem cell-derived neuronal cell, a stem cell-derived muscle cell, a stem cell-derived liver cell, a stem cell-derived pancreatic cell, a stem cell-derived lung cell, a stem cell-derived adipocyte, a stem cell-derived cardiomyocyte, a stem cell-derived hematopoietic cell, a stem cell-derived keratinocyte, a stem cell-derived epithelial cell, a stem cell-derived endothelial cell, a stem cell-derived astrocyte, a stem cell-derived oligodendrocyte, a stem cell-derived glial cell, a stem cell-derived retinal cell, a stem cell-derived epidermal cell, a stem cell-derived ear cell, a stem cell-derived erythroid cell, a stem cell-derived immune cell, and a stem cell-derived germ cell.
<5> The composition of any one of <1> to <4>, wherein the stem cell-derived cell is a stem cell-derived neuronal cell.
<6> The composition of any one of <1> to <5>, wherein modulating the function of the stem cell-derived cell is controlling differentiation of the stem cell.
<7> The composition of any one of <1> to <5>, wherein modulating the function of the stem cell-derived cell is enhancing synaptogenesis, improving neuronal morphogenesis, improving neuronal growth, and/or improving neuronal activity.
<8> The composition of any one of <1> to <7>, wherein the stem cell is an induced pluripotent stem cell (iPSC) and/or an embryonic stem cell (ESC).
<9> The composition of any one of <1> to <8>, wherein the composition is a pharmaceutical composition for treating a disease and/or a disorder associated with a nervous system.
<10> The composition of <9>, wherein the disease and/or the disorder associated with a nervous system comprises Alzheimer's disease, dementia, schizophrenia, autism, ADHD, Parkinson's, multiple sclerosis, epilepsy, Charcot-Marie-Tooth disease, Retinitis, metabolic neurodegenerative disease, dystonia, intellectual disability, deafness, spinocerebellar ataxia, sleep disorders, cortical dysplasia, dyslexia, microphthalmia, leukodystrophy, and/or dyskinesia.
<11> A method for identifying a protein in a population of proteins in a tissue and/or an organ and/or an in vitro cell culture and/or purified subcellular fraction, comprising the following steps (a) to (c):
(a) digesting the protein in the population of proteins with one or more enzymes;
(b) repeating the step (a) one or more times to produce peptides; and
(c) separating the peptides into fractions.
<12> The method of <11>, further comprising detecting and sequencing the peptides by using mass spectrometry.
<13> The method of <11> or <12>, wherein the enzymes comprise lys-C and/or trypsin.
<14> The method of any one of <11> to <13>, wherein the steps (a) and (b) consist of sequential protein digestion steps with LysC and trypsin-LysC in combination.
<15> The method of <14>, wherein the sequential protein digestion steps comprise a first step of protein digestion with LysC and a second protein digestion with trypsin and LysC at the same time.
<16> The method of any one of <11> to <15>, wherein the separation in the step (c) is based on electrostatic repulsion/hydrophilic interaction chromatography (ERLIC).
<17> The method of <16>, further comprising performing reverse phase chromatography (RPC) of each of the ERLIC fractions.
<18> A method for quantifying a target protein in a tissue and/or an organ and/or an in vitro cell culture and/or purified subcellular fraction, comprising the following steps (d) to (i):
(d) selecting one specific protein from the proteins identified by the method of any one of <11> to <17>;
(e) identifying a proteotypic peptide within the specific protein based on the peptides detected and sequenced by the method of <12>;
(f) labelling at least one amino acid within the proteotypic peptide with an isotope to produce an isotope-labeled proteotypic peptide;
(g) adding a predetermined quantity of the isotope-labeled proteotypic peptide to a fraction from a sample;
(h) performing mass spectrometry for the fraction; and
(i) comparing a signal from a target peptide derived from the target protein and a signal from the isotope-labeled proteotypic peptide added to the fraction in the step (g).
<19> The method of <18>, wherein the in vitro cell culture is an induced pluripotent stem cell (iPSC) culture, an embryonic stem cell (ESC) culture, and/or an iPSC- or ESC-derived cell culture.
<20> The method of <19>, further comprising identifying a protein, the expression level of which is larger or lower in the iPSC- or ESC-derived cell culture than in the iPSC or ESC culture.
<21> The method of <20>, further comprising identifying a ligand or transcription factor against the identified protein, the expression level of which is larger or lower in the iPSC- or ESC-derived cell culture than in the iPSC or ESC culture.
<22> The method of <20> or <21>, wherein the identified protein is a receptor.

### In Some Embodiments, the Present Invention Relates to the Following:

[1] A method for identifying a protein in a population of proteins in a tissue and/or an organ and/or an in vitro cell culture and/or purified subcellular fraction, comprising the following steps (a) to (c):
(a) digesting the protein in the population of proteins with one or more enzymes;
(b) repeating the step (a) one or more times to produce peptides; and
(c) separating the peptides into fractions.
[2] The method of [1], further comprising isolating the protein in the population of proteins from a tissue and/or an organ and/or an in vitro cell culture and/or purified subcellular fraction.
[3] The method of [1] or [2], further comprising denaturing the protein in the population of proteins.
[4] The method of any of [1] to [3], wherein a mass spectrometry is performed after the step (c).
[5] The method of any of [1] to [4], further comprising detecting and sequencing the peptides by using a mass spectrometry.
[6] The method of any of [1] to [5], further comprising identifying the protein in the population of proteins.
[7] The method of any of [1] to [6], wherein the tissue and/or the organ comprises a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[8] The method of any of [1] to [7], wherein the in vitro cell culture is associated with a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[8bis] The method of any of [1] to [7], wherein the purified subcellular fraction is associated with a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[9] The method of any of [1] to [8bis], wherein the tissue and/or the organ comprises a nervous system.
[10] The method of any of [1] to [9], wherein the in vitro cell culture is associated with a nervous system.
[10bis] The method of any of [1] to [9], wherein the purified subcellular fraction is associated with a nervous system.
[11] The method of any of [1] to [10bis], wherein the population of proteins is defined as a population of proteins present in a synapse.
[12] The method of any of [1] to [11], wherein the population of proteins is defined as a population of proteins present in a synaptic vesicle.
[12bis] The method of any of [1] to [11], wherein the population of proteins is defined as a population of proteins present in a synaptic mitochondria, presynaptic membrane, active zone, postsynaptic density, synaptic cleft.
[13] The method of any of [1] to [12bis], wherein the enzymes are defined as proteinase, protease, and/or proteolytic enzyme.
[14] The method of any of [1] to [13], wherein the enzymes comprise lys-C, trypsin, Lys-N, Asp-N, Arg-C, Glu-C, chymotrypsin, thermolysin, pepsin, elastase, and/or factor Xa.
[15] The method of any of [1] to [14], wherein the enzymes comprise lys-C and/or trypsin.
[16] The method of any of [1] to [15], wherein the separation in the step (c) is based on chromatography.
[17] The method of any of [1] to [16], wherein the separation in the step (c) is based on electrostatic repulsion/hydrophilic interaction chromatography (ERLIC).
[18] The method of [17], wherein the ERLIC is performed by using first and second solvents.
[19] The method of [18], wherein the first and second solvents comprise organic solvent and carboxylic acid.
[20] The method of or [19], wherein the first and second solvents comprise acetonitrile and formic acid.
[21] The method of any of [18] to [20], wherein the first solvent comprises 70 to 100% (v/v) acetonitrile and 0.01 to 1.0% (v/v) formic acid.
[22] The method of any of [18] to [21], wherein the first solvent comprises 90% (v/v) acetonitrile and 0.1% (v/v) formic acid.
[23] The method of any of [18] to [22], wherein the second solvent comprises 10 to 50% (v/v) acetonitrile and 0.01 to 1.0% (v/v) formic acid.
[24] The method of any of [18] to [23], wherein the second solvent comprises 30% (v/v) acetonitrile and 0.1% (v/v) formic acid.
[24bis] The method of any of [18] to [23], wherein the first solvent comprises ammonium hydroxide to adjust the pH at 4.5.
   (This is important to create a pH gradient which also participate to peptide separation)
[25] The method of any of [18] to [24bis], wherein the first solvent comprises 90% (v/v) acetonitrile and 0.1% (v/v) formic acid, and the second solvent comprises 30% (v/v) acetonitrile and 0.1% (v/v) formic acid.
[26] The method of any of [17] to [25], wherein the ERLIC is performed by using a gradient mode.
[27] The method of [26], wherein the gradient mode comprises the following steps (i) and (ii): (i) 1 to 10 min for the first solvent, and (ii) to 1 to 100% for the second solvent in 1 to 30 min.
[28] The method of [27], wherein the step (ii) is repeated one or more times.
[29] The method of any of [26] to [28], wherein the gradient mode comprises the following steps: 1 to 10 min for the first solvent, to 1 to 20% for the second solvent in 1 to 15 min, 1 to 20% for the second solvent to 10 to 40% for the second solvent in 10 to 30 min, 10 to 40% for the second solvent to 50 to 90% for the second solvent in 5 to 25 min, 50 to 90% for the second solvent to 70 to 95% for the second solvent in 1 to 10 min, and 70 to 95% for the second solvent to 90 to 100% for the second solvent in 1 to 10 min.
[30] The method of any of [26] to [29], wherein the gradient mode is followed by wash at 90 to 100% for the second solvent for 1 to 10 min.
[31] The method of [30], wherein the wash is followed by re-equilibration at 90 to 100% for the first solvent for 10 to 30 min.
[32] The method of any of [26] to [31], wherein the gradient mode is performed at a flow rate of 10 to 100 μL/min.
[33] The method of any of [26] to [32], wherein the gradient mode comprises the following steps: 3 min for the first solvent, to 10% for the second solvent in 7 min, 10% for the second solvent to 25% for the second solvent in 24 min, 25% for the second solvent to 70% for the second solvent in 16 min, 70% for the second solvent to 81% for the second solvent in 6 min, 81% for the second solvent to 100% for the second solvent in 3 min, with wash at 100% for the second solvent for 6 min and re-equilibration at 100% for the first solvent for 20 min at a flow rate of 40 μL/min.
[34] A method for identifying a proteotypic peptide, comprising the following steps (d) and (e):
(d) selecting one specific protein from the proteins identified by the method of any of [1] to [33]; and
(e) identifying the proteotypic peptide within said protein based on the peptides detected and sequenced by the method of any of [1] to [33].
[35] The method of [34], wherein the proteotypic peptide is defined as a peptide which has a sequence found in only a single protein and is used to identify said protein in a population of proteins in a tissue and/or an organ and/or an in vitro cell culture and/or purified subcellular fraction.
[36] The method of or [35], wherein the tissue and/or the organ comprises a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[37] The method of any of [34] to [36], wherein the in vitro cell culture is associated with a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[37bis] The method of any of [34] to [36], wherein the purified subcellular fraction is associated with a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[38] The method of any of [34] to [37bis], wherein the tissue and/or the organ comprises a nervous system.
[39] The method of any of [34] to [38], wherein the in vitro cell culture is associated with a nervous system.
[39bis] The method of any of [34] to [38], wherein the purified subcellular fraction is associated with a nervous system.
[40] The method of any of [34] to [39bis], wherein the population of proteins is defined as a population of proteins present in a synapse.
[41] The method of any of [34] to [40], wherein the population of proteins is defined as a population of proteins present in a synaptic vesicle.
[41bis] The method of any of [34] to [40], wherein the population of proteins is defined as a population of proteins present in a synaptic mitochondria, presynaptic membrane, active zone, postsynaptic density, synaptic cleft.
[42] The method of any of [34] to [41bis], wherein an amino acid sequence of the proteotypic peptide is at least 4 amino-acid length.
[43] A method for producing an isotope-labeled proteotypic peptide, comprising the following steps (f) and (g):
(f) identifying the proteotypic peptide by using the method of any of [34] to [42]; and
(g) labelling at least one amino acid within the peptide with an isotope.
[44] The method of [43], wherein the isotope-labeled proteotypic peptide is defined as a peptide which has a sequence found in only a single protein and is used to identify said protein in a population of proteins in a tissue and/or an organ and/or an in vitro cell culture and/or purified subcellular fraction and is isotope-labeled.
[45] The method of or [44], wherein lysine and/or arginine within the peptide is labeled with an isotope.
[46] An isotope-labeled proteotypic peptide produced by the method of any of to [45].
[47] An isotope-labeled peptide derived from a single trace protein in a population in a tissue and/or an organ and/or an in vitro cell culture and/or purified subcellular fraction for use in quantifying the trace protein in a tissue and/or an organ and/or an in vitro cell culture and/or purified subcellular fraction, wherein at least one amino acid within the peptide is isotope-labeled.
[48] The isotope-labeled peptide of [47], wherein the trace protein is identified by the method of any of [1] to [33].
[49] The isotope-labeled peptide of or [48], wherein an amino acid sequence of the isotope-labeled peptide is detected by the method of any of [1] to [33].
[50] The isotope-labeled peptide of any of [47] to [49], wherein an amino acid sequence of the isotope-labeled peptide is at least 4 amino-acid length.
[51] A peptide derived from a single trace protein in a population in a tissue and/or an organ and/or an in vitro cell culture and/or purified subcellular fraction.
[52] The peptide of [51], wherein the trace protein is identified by the method of any of [1] to [33].
[53] The peptide of or [52], wherein an amino acid sequence of the peptide is detected by the method of any of [1] to [33].
[54] The peptide of any of [51] to [53], wherein an amino acid sequence of the peptide is at least 4 amino-acid length.
[55] The peptide of any of [47] to [54], wherein the tissue and/or the organ comprises a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[56] The peptide of any of [47] to [55], wherein the in vitro cell culture is associated with a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[56bis] The peptide of any of [47] to [55], wherein the purified subcellular fraction is associated with a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[57] The peptide of any of [47] to [56bis], wherein the tissue and/or the organ comprises a nervous system.
[58] The peptide of any of [47] to [57], wherein the in vitro cell culture is associated with a nervous system.
[58bis] The peptide of any of [47] to [57], wherein the purified subcellular fraction is associated with a nervous system.
[59] The peptide of any of [47] to [58bis], wherein the population of proteins is defined as a population of proteins present in a synapse.
[60] The peptide of any of [47] to [59], wherein the population of proteins is defined as a population of proteins present in a synaptic vesicle.
[61] A method for quantifying a target protein in a tissue and/or an organ and/or an in vitro cell culture and/or purified subcellular fraction, comprising the following steps (h) to (j):
(h) adding predetermined quantity of the peptide of any of [46] to [60] to a fraction from a sample;
(i) performing a mass spectrometry for the fraction; and
(j) comparing a signal from a target peptide derived from the target protein and a signal from the peptide of any of [46] to [60] added to the fraction in the step (h).
[62] The method of [61], further comprising calculating an m/z spectral area of the signal from the target peptide and that of the signal from the peptide of any of [46] to [60] added to the fraction in the step (h).
[63] The method of or [62], wherein the sample is from a subject suffering from a disease and/or a disorder.
[64] The method of [63], wherein the disease and/or the disorder is those associated with a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[65] The method of or [64], wherein the disease and/or the disorder comprises a brain cognitive, motor, sensory processing, neurodegenerative diseases, and/or neurodevelopmental diseases.
[66] The method of any of [63] to [65], wherein the disease and/or the disorder associated with a nervous system comprises Alzheimer's disease, dementia, schizophrenia, autism, ADHD, Parkinson's, multiple sclerosis, Epilepsia, Charcot-Marie-Tooth disease, Retinitis, metabolic neurodegenerative disease, dystonia, mental retardation, deafness, spinocerebellar ataxia, sleep disorders, cortical dysplasia, dyslexia, microphthalmia, leukodystrophy, and/or dyskinesia.
[67] The method of any of [61] to [66], further comprising the following steps (k) and (l):
(k) performing a mass spectrometry with a target peptide derived from the target protein and the peptide of any of [46] to [60] at predetermined concentration levels to create a calibration curve, wherein the target peptide has the amino acid sequence same as that of the peptide of any of [46] to [60] and not labelled; and
(l) obtaining a quantitative value from the area ratio using the calibration curve.
[68] A method for diagnosing a disease and/or a disorder in a subject, comprising comparing a profile of a target protein in a sample obtained from a subject having a disease and/or a disorder with that of a healthy control, wherein the profile comprises an expression level of the target protein quantified by the method of any of [61] to [67].
[69] The method of [68], wherein the disease and/or the disorder is those associated with a nervous system, a heart, a lung, a liver, a spleen, a kidney, a stomach, a small intestine, a large intestine, a gall, a bladder, a skin, a muscle, a blood, a lymphatic system, an adrenal gland, a testis, an ovary, a rectum, a pancreas, an esophagus, a thyroid gland, a bone marrow, a retina, a placenta, and/or body fluids.
[70] The method of [68] or [69], wherein the disease and/or the disorder comprises a brain cognitive, motor, sensory processing, neurodegenerative diseases, and/or neurodevelopmental diseases.
[71] The method of any of [68] to [70], wherein the disease and/or the disorder associated with a nervous system comprises Alzheimer's disease, dementia, schizophrenia, autism, ADHD, Parkinson's, multiple sclerosis, Epilepsia, Charcot-Marie-Tooth disease, Retinitis, metabolic neurodegenerative disease, dystonia, mental retardation, deafness, spinocerebellar ataxia, sleep disorders, cortical dysplasia, dyslexia, microphthalmia, leukodystrophy, and/or dyskinesia.
[72] A composition comprising the peptide of any of [46] to [60].
[73] A method for identifying a protein in a population of proteins in a sample, comprising the following steps (m) to (o): (m) digesting the protein in the population of proteins with one or more enzymes; (n) repeating the step (m) one or more times to produce peptides; and (o) separating the peptides into fractions.
[74] A method for quantifying a target protein in a sample, comprising the following steps (p) to (r):
(p) adding predetermined quantity of the peptide of any of [46] to [60] to a fraction from a sample;
(q) performing a mass spectrometry for the fraction; and
(r) comparing a signal from a target peptide derived from the target protein and a signal from the peptide of any of [46] to [60] added to the fraction in the step (p).
[75] A peptide having a sequence defined in the list of peptide information, which is derived from a single protein.
[76] The peptide of [75], wherein the single protein is present in a synapse.
[77] The peptide of [75], wherein the single protein is present in a synaptic vesicle.
[78] The method of any of [61] to [67], wherein the in vitro cell culture is an induced pluripotent stem cell (iPSC) culture, an embryonic stem cell (ESC) culture, and/or an iPSC- or ESC-derived cell culture.
[79] The method of [78], further comprising identifying a protein, the expression level of which is larger or lower in the iPSC- or ESC-derived cell culture than in the iPSC or ESC culture.
[80] The method of [79], further comprising identifying a ligand or transcription factor against the protein, the expression level of which is larger or lower in the iPSC- or ESC-derived cell culture than in the iPSC or ESC culture.
[81] The method of any of [78] to [80], wherein the iPSC- or ESC-derived cell culture is selected from the group consisting of an iPSC- or ESC-derived neuronal cell culture, an iPSC- or ESC-derived muscle cell culture, an iPSC- or ESC-derived liver cell culture, an iPSC- or ESC-derived pancreatic cell culture, an iPSC- or ESC-derived lung cell culture, an iPSC- or ESC-derived adipocyte culture, an iPSC- or ESC-derived cardiomyocyte culture, an iPSC- or ESC-derived hematopoietic cell culture, an iPSC- or ESC-derived keratinocyte culture, an iPSC- or ESC-derived epithelial cell culture, an iPSC- or ESC-derived endothelial cell culture, an iPSC- or ESC-derived astrocyte culture, an iPSC- or ESC-derived oligodendrocyte culture, an iPSC- or ESC-derived glial cell culture, an iPSC- or ESC-derived retinal cell culture, an iPSC- or ESC-derived epidermal cell culture, an iPSC- or ESC-derived ear cell culture, an iPSC- or ESC-derived erythroid cell culture, an iPSC- or ESC-derived immune cell culture, and an iPSC- or ESC-derived germ cell culture.
[82] The method of any of [78] to [81], wherein the iPSC- or ESC-derived cell culture is an iPSC- or ESC-derived neuronal cell culture.
[83] The method of any of [79] to [82], wherein the protein, the expression level of which is larger in the iPSC- or ESC-derived cell culture than in the iPSC or ESC culture, is selected from the group consisting of FGFR2, FGFR3, TrKB, GDNF receptor, and CNTFR.
[84] A ligand identified by the method of any of [80] to [83].
[85] The ligand of [84], wherein the ligand is selected from the group consisting of FGF3, FGF7, FGF10, FGF22, FGF8, FGF9, FGF16, FGF17, FGF18, FGF20, BDNF, GDNF, NRTN, PSPN, and CNTF.
[86] The ligand of or [85], wherein the ligand is FGF22 or the combination of (i) BDNF and GDNF, (ii) BDNF, CNTF, and GDNF, (iii) BDNF, CNTF, GDNF, and FGF16, or (iv) BDNF, CNTF, GDNF, FGF16, and FGF22.
[87] A composition comprising the ligand of any of [84] to [86].
[88] The composition of [87], wherein the composition is a pharmaceutical composition.
[89] The composition of [88], further comprising a pharmaceutically acceptable carrier.
[90] The composition of any of [87] to [89], wherein the composition is for modulating the function of the iPSC- or ESC-derived cell culture.
[91] The composition of [90], wherein the iPSC- or ESC-derived cell culture is selected from the group consisting of an iPSC- or ESC-derived neuronal cell culture, an iPSC- or ESC-derived muscle cell culture, an iPSC- or ESC-derived liver cell culture, an iPSC- or ESC-derived pancreatic cell culture, an iPSC- or ESC-derived lung cell culture, an iPSC- or ESC-derived adipocyte culture, an iPSC- or ESC-derived cardiomyocyte culture, an iPSC- or ESC-derived hematopoietic cell culture, an iPSC- or ESC-derived keratinocyte culture, an iPSC- or ESC-derived epithelial cell culture, an iPSC- or ESC-derived endothelial cell culture, an iPSC- or ESC-derived astrocyte culture, an iPSC- or ESC-derived oligodendrocyte culture, an iPSC- or ESC-derived glial cell culture, an iPSC- or ESC-derived retinal cell culture, an iPSC- or ESC-derived epidermal cell culture, an iPSC- or ESC-derived ear cell culture, an iPSC- or ESC-derived erythroid cell culture, an iPSC- or ESC-derived immune cell culture, and an iPSC- or ESC-derived germ cell culture.
[92] The composition of [90] or [91], wherein the iPSC- or ESC-derived cell culture is an iPSC- or ESC-derived neuronal cell culture.
[93] The composition of [92], wherein the modulating the function of the iPSC- or ESC-derived cell culture is enhancing synaptogenesis in the iPSC- or ESC-derived neuronal cell culture.
[94] The composition of any of [87] to [93], wherein the composition is for treating a disease and/or a disorder associated with a nervous system in a subject.
[95] The composition of [94], wherein the disease and/or the disorder associated with a nervous system comprises Alzheimer's disease, dementia, schizophrenia, autism, ADHD, Parkinson's, multiple sclerosis, Epilepsia, Charcot-Marie-Tooth disease, Retinitis, metabolic neurodegenerative disease, dystonia, mental retardation, deafness, spinocerebellar ataxia, sleep disorders, cortical dysplasia, dyslexia, microphthalmia, leukodystrophy, and/or dyskinesia.
[96] A method for modulating the function of the iPSC- or ESC-derived cell culture, comprising adding the ligand of any of [84] to [86] or the composition of any of [87] to [95] to the iPSC- or ESC-derived cell culture.
[97] The method of [96], wherein the iPSC- or ESC-derived cell culture is selected from the group consisting of an iPSC- or ESC-derived neuronal cell culture, an iPSC- or ESC-derived muscle cell culture, an iPSC- or ESC-derived liver cell culture, an iPSC- or ESC-derived pancreatic cell culture, an iPSC- or ESC-derived lung cell culture, an iPSC- or ESC-derived adipocyte culture, an iPSC- or ESC-derived cardiomyocyte culture, an iPSC- or ESC-derived hematopoietic cell culture, an iPSC- or ESC-derived keratinocyte culture, an iPSC- or ESC-derived epithelial cell culture, an iPSC- or ESC-derived endothelial cell culture, an iPSC- or ESC-derived astrocyte culture, an iPSC- or ESC-derived oligodendrocyte culture, an iPSC- or ESC-derived glial cell culture, an iPSC- or ESC-derived retinal cell culture, an iPSC- or ESC-derived epidermal cell culture, an iPSC- or ESC-derived ear cell culture, an iPSC- or ESC-derived erythroid cell culture, an iPSC- or ESC-derived immune cell culture, and an iPSC- or ESC-derived germ cell culture.
[98] The method of [96] or [97], wherein the iPSC- or ESC-derived cell culture is an iPSC- or ESC-derived neuronal cell culture.
[99] The method of [98], wherein the modulating the function of the iPSC- or ESC-derived cell culture is enhancing synaptogenesis in the iPSC- or ESC-derived neuronal cell culture.
[100] A method for treating a disease and/or a disorder associated with a nervous system in a subject, comprising administering an effective amount of the ligand of any of [84] to [86] or the composition of any of [87] to [95] to the subject.
[101] The method of [100], wherein the disease and/or the disorder associated with a nervous system comprises Alzheimer's disease, dementia, schizophrenia, autism, ADHD, Parkinson's, multiple sclerosis, Epilepsia, Charcot-Marie-Tooth disease, Retinitis, metabolic neurodegenerative disease, dystonia, mental retardation, deafness, spinocerebellar ataxia, sleep disorders, cortical dysplasia, dyslexia, microphthalmia, leukodystrophy, and/or dyskinesia.

### Effects of the Invention

According to the present invention, a new approach called ‘Ultra-definition proteomics’ (UD method) for deep subcellular proteome identification and quantification can be provided. We succeeded in dramatically improving the detection sensitivity of mass spectrometry by the UD method and making it possible to detect almost all of synaptic proteins from synapse fraction. By analyzing human neurons induced from patient cell-derived iPS and disease animal model by the UD method, it is possible to capture the changes (increase and decrease, change in distribution, post-translational modifications) of each synaptic molecule comprehensively, and it is expected to be able to understand disease conditions more deeply.

Furthermore, according to the present invention, a composition comprising a ligand for reprogramming stem cell and/or modulating functions of stem cell-derived cell culture is provided. Stem cell reprograming has great potential in applications such as regenerative medicine and drug discovery. Conventional reprogramming methods were made to simply differentiate cells into basic neurons. However, the stem cell programming of the present invention can optimize neuronal morphogenesis and functions. For example, one conventional method is to supplement cell culture medium with BDNF and NT3 growth factors. With our proteomics data, we showed that BDNF receptors TrKB but not NT3 receptors TrKC is expressed in the reprogrammed neurons. Thus, NT3 could be excluded from the medium. Additionally, proteomics data revealed the presence of CNTFR, GDNFR1, GDNFR2, GDNFR3, FGFR2, FGFR3 receptors on reprogrammed neurons. Thus, we match with their ligands (CNTF, GDNF, FGF16, FGF22) by adding those in the medium. As a result, this significantly improved neuronal morphogenesis and enhanced synapse formation. This process can be adapted for further stem cell reprogramming into any cell types.

In addition, according to the present invention, a pharmaceutical composition for treating a disease and/or a disorder associated with a nervous system is provided.

The method of the present invention can conduct comprehensive and quantitative proteomic analysis of cellular proteins. The composition comprising a ligand of the present invention can enhance cellular health of differentiated stem cells, and enhance cellular functions of differentiated stem cells, and can be used for stem cell differentiation, regenerative medicine development, patient-personalized and precision diagnostics.

### BRIEF DESCRIPTION OF THE DRAWINGS

FIGS. 1A and 1B. The low abundant synaptic proteome is strongly related to neurological disorders.
(A) Numbers of synaptic vesicles (SV) proteins identified in Takamori et al Cell 2006 and using the synapse UD proteomics workflow.
(B) Quantitative representation in a rank-abundance plot of the SV proteome. Y-axis: log base 10 of iBAQ score; X-axis: abundance score rank in the proteome. Proteins having disease(s) caused by mutation(s) affecting the gene represented in the database entry are indicated (black circles). Of the ~1,500 reported SV proteins, 210 are genetically associated with distinct brain diseases. Remarkably, a majority of these are low abundant and were ‘invisible’ to previous synapse proteomics studies (Takamori et al Cell 2006; Wilhelm et al Science 2014; Koopmans et al Neuron 2019).

FIGS. 2A-2F. Ultra-definition proteomics tripled the known SV proteome size.
(A) Key steps in the ultra-definition (UD) proteomics method: sequential enzymatic digestion steps followed by orthogonal peptide separations using multiple biophysical properties of amino acids (See also FIG. 6).
(B) Unique peptide coverage by mass spectrometry of the active zone protein Piccolo (highlighted amino acid sequence) in Takamori et al. (2006), HD and UD proteomic methods.
(C) Numbers of proteins identified in the synaptic vesicle (SV) fraction by Takamori et al. (2006) (yellow), HD (white) and UD (grey) methods.
(D) Synaptotagmin family members identified in the SV fraction by Takamori et al. (2006), HD and UD methods.
(E) EM images of the purified synaptosome (P2′) and SV fractions, showing clear vesicles with diameters of ~40 nm. Bottom panels: Representative synaptic structures in the P2′ fraction in EM images, showing intact subsynaptic compartments, as illustrated.
(F) SDS-PAGE profiles of proteins extracted from the P2′ and SV fractions.

FIGS. 3A1-A3, 3B1-B3, and 3C1-C3. UD proteomics unveiled hidden proteins in both high- and low-abundance ranges of the SV proteome.
(A) UD proteomics distinguishes highly homologous protein isoforms, Rab11A and Rab11B. Left panel: Positions of Rab11A and Rab11B in the ranked (iBAQ) abundance curve. Middle panel: Amino acid sequence alignment of Rab11A and Rab11B, showing 91% identity. Right panel: List of unique Rab11 peptides (SEQ ID NOs: 1-17) detected in the SV fraction by HD and UD methods. Rab11A is identified only by UD from a unique peptide at the C-terminal region of Rab GTPase.
(B) Left panel: V-ATPase-related proteins detected in the SV fraction on the ranked (iBAQ) abundance curve. Right illustration: Structural model of the V-ATPase protein set in SVs. Proteins newly detected by UD proteomics are indicated in red.
(C) SV-resident transporter proteins newly detected in the SV fraction by UD proteomics (red), known but missing in previous proteomic studies (purple) in the SV-P2′ volcano plot (left) and Venn diagram (right top). Right bottom panel: Position of the transporters in the ranked (iBAQ) abundance curve of the SV proteome.

FIGS. 4A1-A2, 4B, 4C1A-C1D, 4C2, and 4D1-D2. A previously hidden SV-resident protein shows high amino acid sequence homology among mammals.
(A) ‘Uncharacterized Protein RGD1305455’ (SEQ ID NO: 18; Uniprot ID: A0A0G2KAX2), identified in UD proteomics from unique peptides (highlighted within amino acid sequence) and its position in the ranked (iBAQ) abundance plot (lower panel). No unique peptide could be detected with the HD method.
(B) SV-resident position of RGD1305455 in the SV-P2′ volcano plot.
(C) Amino acid sequence comparison of RGD1305455 homologs in various animal species. Black and grey shading indicates identical and similar amino acids, respectively. Dashes represent gaps in sequences. See FIG. 10 for protein accession numbers and reference sequences. Mammal species are framed in dashed red line boxes (left panel) and species pictures with >97% identity (right panel).
(D) Confirmation of the existence of RGD1305455 protein in the SV fraction using a targeted proteomic approach. VLVVEPVK peptide (SEQ ID NO: 19; detected in UD proteomics) was synthesized using C-terminal ‘heavier’ [¹³C₆ ¹⁵N₂] lysine (+8 neutrons=a predefined shift of 8 Da) and was used to track native peptides after mixing with digested SV proteins. Left: MS2 spectra of heavy VLVVEPVK peptide (SEQ ID NO: 19). Right: MS2 spectra of a native peptide detected in the SV fraction that coincides with that of the heavy peptide. Red, blue, and black peaks indicate matched y-ion series, b-ion series, and unmatched ions respectively (upper panels). Amino acid sequences corresponding to the ion fragments (middle panels). Plotted mass errors of detected versus expected peptide fragments (lower panels). Errors of native peptide fragments were all <0.02 Dalton. Expected masses of all fragments are specified in Table S5.

FIGS. 5A1-A8, and 5B. Diversity of neuronal functions and dysfunctions related to the UD-SV proteome.
(A) Functional mosaic of the SV proteome. Each protein detected in the SV fraction by UD proteomics was associated with one or more functional keywords. Top left panel: Sunburst diagrams show distributions of the functional categories (inner circle) and subcategories (outer circle) represented in the total SV fraction proteome and in the SV-resident repertoire. List of functional keywords (left) and subcategories (right) with number of proteins representing each category.
(B) The deep low-abundant SV proteome is related to brain diseases. Proteins detected in the SV fraction having ‘disease(s) caused by mutation(s) affecting the gene represented in the entry’ were marked and their rank in the iBAQ abundance curve is specified. The analysis was performed manually using the Uniprot and GeneCards databases for human diseases. Markers indicate proteins associated with cognitive (purple), motor (red), and/or sensory processing (yellow) disabilities. The vertical dashed line indicates rank 409, the number of proteins identified by a previous SV proteomics study (Takamori et al. 2006). Proteins to the right hand of the dashed line were mostly revealed by UD proteomics.

FIGS. 6A1-A2, 6B1-B3, and 6C. Methodological advancement of SV proteomics
(A) Schematic description of different protocols and MS instruments employed by Takamori et al. (2006), the HD method, and the UD method. Takamori et al and the HD method utilize conventional one-step trypsin digestion and on-line reverse-phase chromatography (RPC), whereas the UD method utilizes sequential protein digestion steps with LysC and trypsin-LysC in combination, followed by off-line electrostatic repulsion-hydrophilic interaction chromatography (ERLIC) and on-line RPC of each of the ERLIC fractions. This protocol enabled identification of 4,439 proteins comprising 87,931 unique peptides (right panels) in the purified synaptosomal fraction (P2′). This outnumbers those identified by the HD methods by 2.5 and 3.7 times, respectively.
(B) ERLIC fractions resolved by C₁₈-reverse phase chromatography (RPC). Orthogonal peptide separation with a mechanism based on multiple biophysical properties of amino acids maximizes separation of peptides with similar masses, but different compositions and sequences. ERLIC separates peptides according to their charges, polarities, pls, orientations, post-translational modifications (e.g. phosphorylation), and RPC, according to hydrophobicity. A small unmasked peak in a square contains many peptides that were analyzed by LC-MS/MS and sequenced for an in-depth proteome identification.
(C) Number and distribution of unique peptides identified across the 24 ERLIC fractions (F) in the P2′ sample. The highest numbers of unique peptides were detected in F7-9 and F12-17.

FIG. 7. Absolute quantity and copy number per SV for selected proteins. Absolute quantification was performed by mixing known quantities of heavy-isotope synthetic peptides (0.1 mg) with 50 mg of digested SV proteins. The exact quantity of the peptide of interest (native) was calculated using the intensity of the heavy-isotope-labeled standard peptide (heavy): Absolute quantity (mg)=0.1× (native/heavy peak areas)

Average copy number of a protein per SV (#/SV) was estimated using synaptotagmin 1 (Syt1), a transmembrane SV-resident protein of molecular weight (MW)=47 kDa, as a reference (15 Syt1 per SV, estimated by Takamori et al., 2006): Protein #/SV=absolute quantity/MW×[(Syt1 #/SV)×(Syt1 MW)/(Syt1 absolute quantity)].

FIG. 8. Transporters and their putative substrates newly detected as SV-residents using UD proteomics.

FIG. 9. Reference sequences and accession numbers used for the amino acid sequence comparison of RGD1305455 homologs.

FIGS. 10A and 10B. Tables of fragment ion masses expected from heavy and native VLVVEPVK peptides (SEQ ID NO: 19).

Masses were generated in silico with PEAK (v7, Bioinformatics Solutions Inc.). Colored masses indicate those from matched ion MS2 spectra observed in our experiments (FIG. 4C). Note that detected y-ions masses (in red) that include the C-terminal amino acid are 8 Daltons heavier in the heavy peptide (upper table) compared to the native peptide (lower table).

FIG. 11. Heavy peptides used to confirm the presence of newly identified SV-resident proteins (SEQ ID NOs: 19-34).

Peptide tracking experiments using these synthesized heavy peptides, as described in FIG. 4C gave uniformly positive results, providing additional evidence of a hitherto undetected SV proteome revealed by UD proteomics.

FIG. 12. Proteomics-based optimization of human iPS cells reprogramming into much healthier and more ‘connected’ neurons.

FIGS. 13A and 13B. Rapid and efficient reprogramming of patient-derived IPS cells into neurons.
A) DIC image of iPS cells generated from patient, depicting the flat colony structure with cobblestone morphology. Scale bar: 100 μm.
B) DIC image of patient's iPSCs differentiated into neurons (iN), 19 days after reprogramming with lentiviral-based delivery of Neurogenin-2. iN cells show extensive features of neuronal morphology, such as branching axons and dendrites. Scale bar: 100 μm.

FIG. 14A-14E. The differentiated neurons (iN) in culture are making functional synapses.
A) DIC image of human ipsc-derived neurons (iN) in culture of DIV25. Scale bar: 100 μm.
B) Immunofluorescence imaging of iN at DIV25 (neuron-specific marker MAP2 (green); DAPI-stained cell nuclei (blue); Scale bar: 50 μm).
C) Immunofluorescence imaging showing iN synapses (synaptic marker synaptophysin (green); neurite marker MAP2 (white); DAPI cell nuclei (blue); Scale bar: 10 μm).
D) Action potentials induced in iN by current injection demonstrating normal basic neuronal membrane properties of iN.
E) Spontaneous synaptic currents in iN (black trace) abolished by addition of glutamate neurotransmitter receptor inhibitor CNQX (red trace) demonstrating the formation of functional excitatory synapses in iN cultures.

FIG. 15A-15C. UD proteomics unveils a large number of hidden proteins in human iPS and induced neuronal (iN) cells.

Mass spectrometry-based proteomics was conducted using HD versus US workflows. HD proteomics seems to be limited in both iPS and iN cells to ~4,000 identified protein species, whereas UD proteomics unveils a significantly larger number of proteins.

Many of the proteins introduced in the figures remain ‘hidden’ in the samples when using HD proteomics.

(‘HD proteomics’=conventional method prior to the use of highly sensitive Orbitrap Fusion Lumos MS device (Thermo). ‘UD proteomics’=the method of the present invention (Taoufiq et al PNAS 2020) prior to the use of highly sensitive Orbitrap Fusion Lumos MS device (Thermo))

FIG. 16A-16E. UD proteomics successfully reprogramming of psychiatric patient's stem cells into neurons.

FIG. 17A-17G. UD proteomics used to optimize iPSC-derived neuronal cultures.

FIG. 18A-18H. Proteomics-based receptor-ligand matching optimizes human iNeurons growth and synaptogenesis.

FIG. 19A-19H. UD proteomic information used to optimize iPSC-derived neuronal cultures.

FIG. 20A-20C. A BDNF-CNTF-GDNF cocktail of growth factors enhances synaptogenesis in patients' iPSC-derived neurons.

FIGS. 21A, 21B1-B2, and 21C. A BDNF-CNTF-GDNF-FGF16-FGF22 cocktail of growth factors greatly enhances synaptogenesis in patients' iPSC-derived neurons.

FIG. 22. UD Proteomics-based receptor-ligand matching optimizes human iNeurons growth and synaptogenesis.

FIG. 23. UD proteomics conduction on nuclear proteins iPS vs iN.

FIG. 24. Ultra-Definition (UD) Proteomics-based optimization of human patients' iPS cells differentiation.

FIG. 25. Key aspects of UD proteomics-based optimization of stem cell differentiation.

FIGS. 26A, 26B, and 26C. Key aspects of UD proteomics-based optimization of stem cell differentiation: Step 1.

FIG. 27. Key aspects of UD proteomics-based optimization of stem cell differentiation: Step 2.

FIG. 28. Proteomics-based receptors-ligands matching.

FIGS. 29A1-A3, 29B, and 29C1 and C2. A breakthrough culture medium recipe makes stem cells (e.g. psychiatric patients) differentiating into much healthier, more ‘connected’, and more active neurons.

FIG. 30. Nuclear UD proteomics-based ‘precision differentiation’ of human iPS cells.

FIG. 31A, 31B, 31C1-C3. Bio chemical purification and proteomics analysis of nuclei from iPS cells.

FIGS. 32A1-A3 and 32B1-B2. Optimization of nuclear protein extraction.

FIGS. 33A and 33B. Identification of new potential master transcription factors for neuronal differentiation.

FIGS. 34A and 34B. Proteomic control of the newly identified neuronal transcription factors using IPSC-derived cardiomyocytes (iCM).

FIGS. 35A, 35B, and 35C. Identification confirmation of potential master transcription factors for neuronal differentiation.

FIGS. 36A, 36B, and 36C. Volcano plots summary.

FIG. 37. UD-proteomics optimization of human iPSC-T cell differentiation as an example.

### DETAILED DESCRIPTION OF INVENTION

The term “reprogramming”, as used herein, is used to refer to a process of changing one cell fate to another, for example, converting a mature differentiated cell into a less-committed precursor (for example, stem cells) or converting a less-committed precursor (for example, a stem cell) to a differentiated cell. That is, the term “reprogramming” may mean controlling differentiation. Our approach for optimizing iPSC-derived T-cell differentiation and for enhancing the differentiated T-cell functional attributes (e.g. cytokine secretion). First, we will use our UD proteomics to identify and quantify all proteins extracted from cell membranes and nuclei of iPSCs versus differentiated T-cells and (if available) versus blood T-cells. The generated data will then serve our two following strategies:

### 1) UD Proteomics-Based Receptor-Ligand Matching:

Create proprietary culture medium recipes, based on the presence, levels and/or absence of cell membrane receptors (e.g. growth factor receptors), by supplementing the culture medium with a combination of matching ligands.

### 2) T-Cell Nuclear UD Proteomic Analyses:

Identify T-cell-type-specific ‘master transcription factors’ by comparing the nuclear proteomes of iPSCs, differentiated T-cells, and (if available) blood T-cells. Deliver by viral vectors the identified factors to iPSC and determine those that contribute the most to developmental maturity. Expected outcome is:
Enhanced cellular health of differentiated T cells (e.g. speed of differentiation).
Enhanced cellular functions of differentiated stem cells (e.g. immune cell stimulation and secreted cytokines).

We believe the project outcome will support and facilitate the development of next-gen therapeutics such as cancer immunotherapy (FIG. 37).

A New Workflow with Enhanced Peptide Recovery and Separation Greatly Extended Synaptic Proteome Coverage

A new workflow was developed to increase coverage of protein-specific sequences or ‘unique peptides’ prior to MS identification. First, to increase the number of accessible cleavage sites, we introduced Lys-C treatments before and during tryptic digestion (FIGS. 2A and 6). Second, to improve separation of the peptides, we introduced off-line fractionation using electrostatic repulsion-hydrophilic interaction chromatography (ERLIC), based on their charges, polarities, pHi, post-translational modifications, and orientations (13, 14), prior to conventional hydrophobicity-based reverse-phase chromatography (RPC).

To evaluate the contribution of this new workflow to greater protein coverage, we also ran a conventional protein digestion-peptide separation protocol combined with modern mass spectrometer (Q-Exactive Plus) analyses, which we designated as ‘high-definition’ (HD) method, whereas we refer to our new workflow as ‘ultra-definition’ (UD) method (FIGS. 2B and 6). The UD-based proteomics revealed 1,466 proteins in the SV fraction (FIG. 2C). This is twice as many as with the HD method (766), and more than three times as many as previously reported (2). The increased sensitivity of the UD method is also evident from the recovery of unique peptides of individual proteins. For instance, 116 unique peptides were identified for the large AZ protein, Piccolo, whereas the HD method recovered only 14, and only 1 was identified in the previous study (2) (FIG. 2B). The UD method increased not only the size of the SV proteome, but also the number of isoforms identified within individual protein families, such as the synaptotagmins (FIG. 2D), for which most known family members were detected (13/15 and extended-Syt1). The newly detected isoforms included Syt7, which was recently found to regulate multiple modes of neurotransmitter release (8-10). In contrast, the HD method added only one Syt isoform to the previous SV proteome (2).

As expected, the UD method also detected a much greater number of proteins (4,439) in synaptosomal fractions (P2′) than the HD method (1,790) (See FIG. 6A), indicating that the resolving power of the UD method is based upon improved workflow prior to MS analysis (See FIG. 6). Note that each sample used for our MS analyses was checked by electron microscopy and electrophoresis. Typical synaptosomal profiles were observed in P2′ samples, whereas uniform vesicle structures of 40-50 nm in diameter predominated SV fractions (FIG. 2E). Proteins extracted from the P2′ and SV fractions showed distinct SDS-PAGE profiles (FIG. 2F).

### Improved Quantification Revealed Synaptic Organization and Diversity of the SV Proteome

In quantitative mass spectrometry, protein abundance can be determined using intensity-based absolute quantification (iBAQ), a label-free approach in which the summed intensities of all unique peptides of a protein are divided by the total number of unique peptides detected. Thus, the increased peptide recovery achieved with the UD method is expected to improve the accuracy of protein quantification. To test this assumption, we performed immunoblot analyses for 41 proteins in the fractions during SV purification and compared with the quantification profiles of the HD and UD methods. As expected, proteins located at the postsynaptic side or in the synaptic cleft were found in the P2′ fraction, but not in the SV fraction, both in immunoblot and MS analyses. Proteins residing on SVs were found at higher levels in the SV than the P2′ fraction, in both western and UD analyses. In contrast, the HD method failed to detect some SV proteins in P2′. Similar inconsistencies between HD iBAQ data and immunoblot profiles were found for proteins in AZ, presynaptic membrane, cytoplasm, altogether in 30% (13/41) of cases. These results highlight the importance of the UD workflow for quantitative proteomics.

SVs are purified from synaptosomes (P2′), which contain all SV proteins, whereas SVs may not contain proteins from other synaptic compartments. Of 4,424 proteins in P2′ fraction, 3,005 were detected only in P2′, including postsynaptic and mitochondrial proteins. Of 1,466 SV proteins, 1,419 were detected in P2′. The remaining 47 SV proteins were of low abundance, including VGLUT3, a vesicular glutamate transporter isoform present in a limited set of CNS synapses. To evaluate possible contamination of postsynaptic proteins into SV fraction, we have referred to the Synaptic Gene Ontologies (SynGO) resource (15). Of all SV fraction proteins, 97 (7%) are annotated as postsynaptic proteins, but 47 out of 97 proteins are reportedly present and function in presynaptic compartments. Thus, contamination of postsynaptic proteins in SV proteome seems minor within the proteins detected in the SynGO database.

To distinguish SV residents from proteins transiently interacting (‘visitors’) with SVs, we determined the iBAQ ratio SV/P2′ in a volcano plot. Of 1,466 SV proteins, 134 had an SV/P2′ ratio significantly higher than 2 (p<0.05). We used this criterion to define the bona-fide ‘SV-resident’ protein group. It comprised all previously established SV proteins (2, 16, 17), as well as novel and hitherto uncharacterized proteins (see below). On the other hand, a majority of the 1,466 proteins had an SV/P2′ ratio lower than 1, suggesting that these occasionally interact with SVs. We defined them as potential SV-visitor proteins. This repertoire contains (i) cytosolic proteins, such as calmodulin, actin, and synaptojanin-1, (ii) AZ proteins, such as Piccolo and Bassoon, and (iii) plasma membrane proteins such as syntaxin-1, all of which interact transiently with SVs, for instance, in the SV trafficking pathway (4, 18, 19). Thus, UD proteomics provide quantitative information to distinguish SV-resident and SV-visitor synaptic protein repertoires.

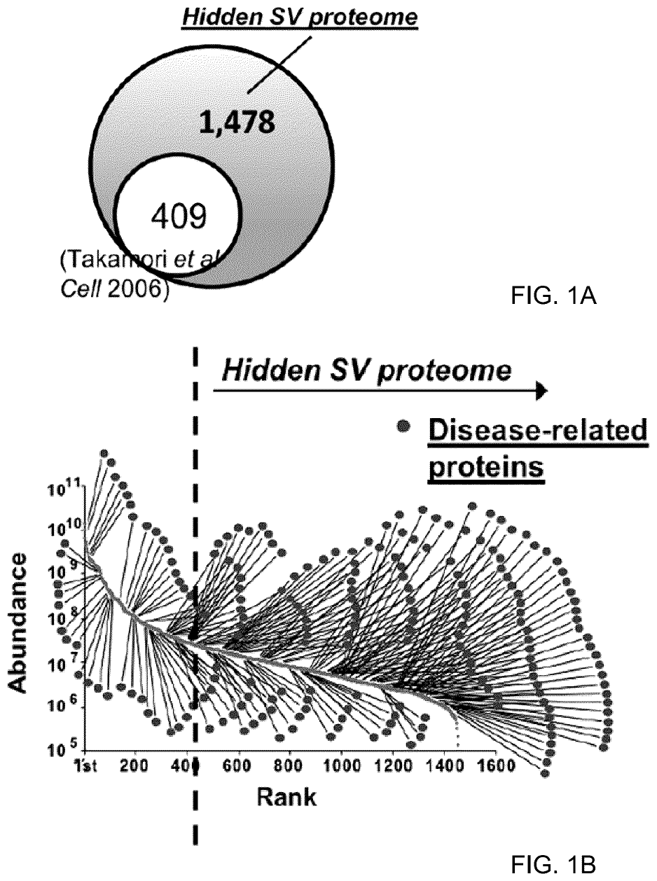

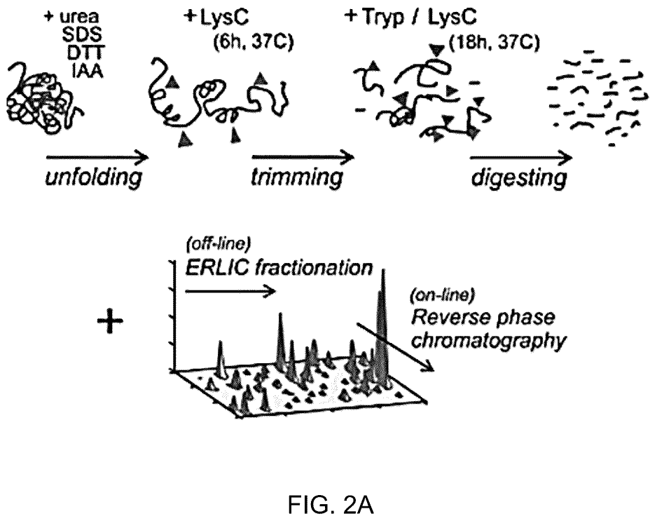

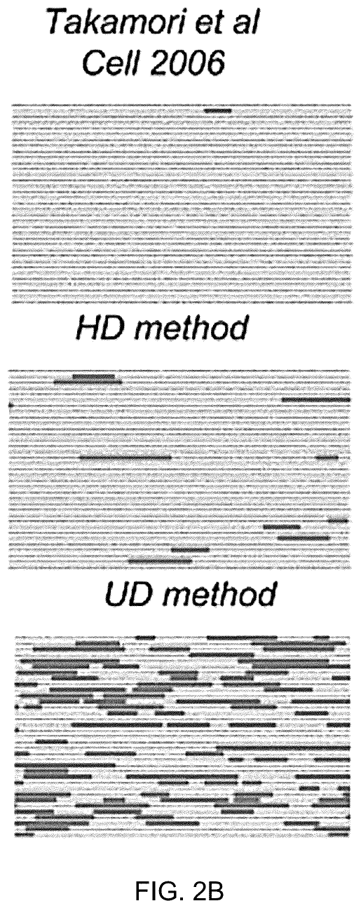

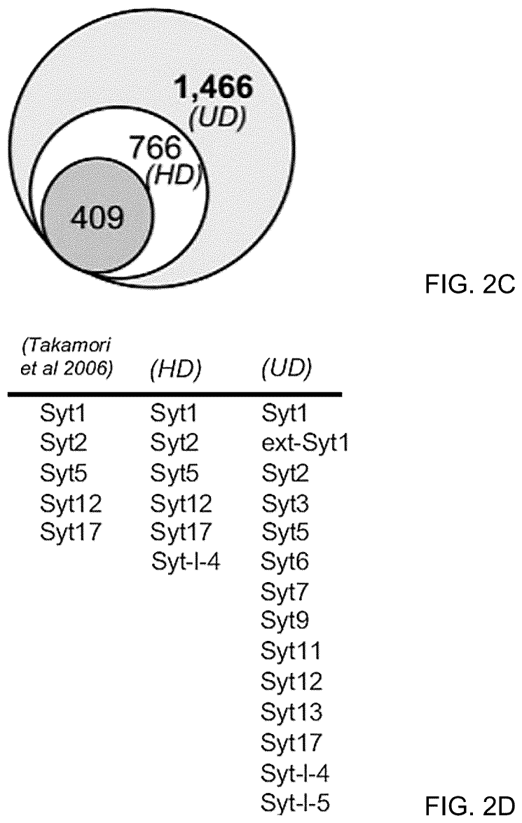

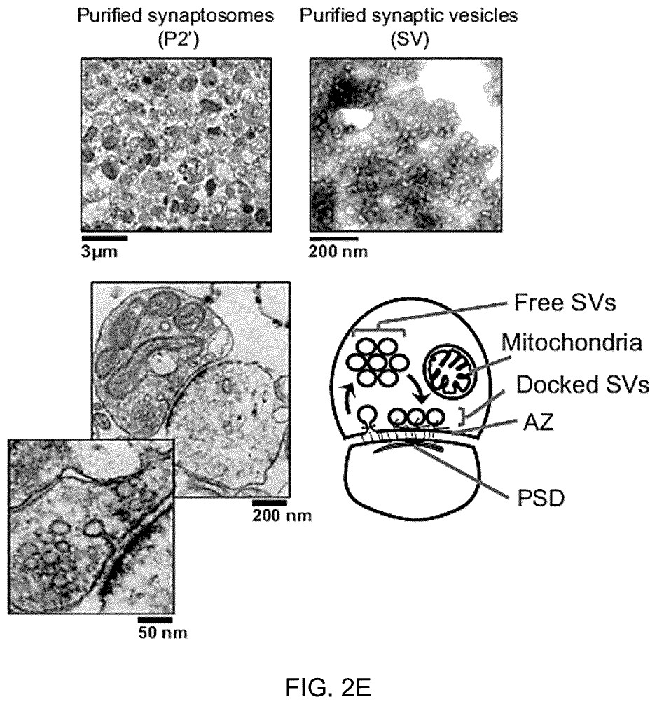

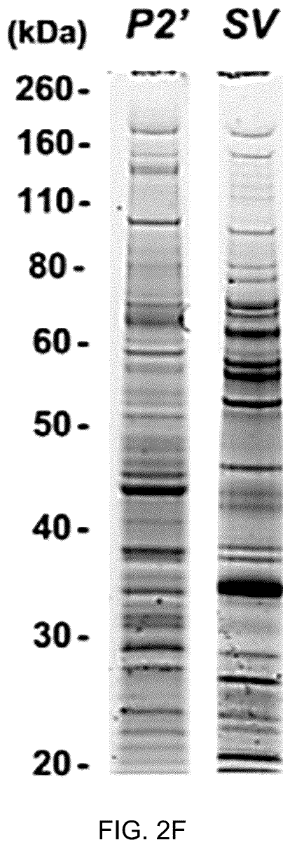

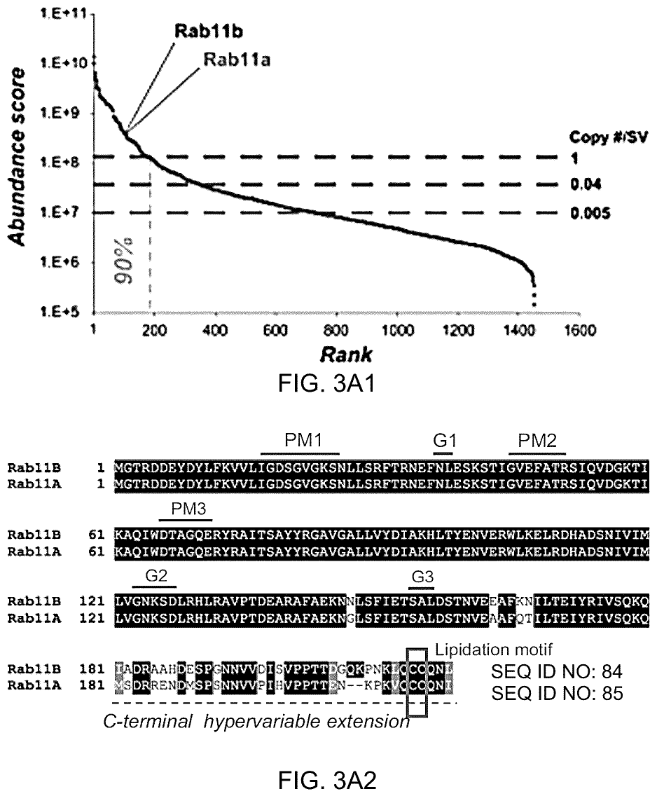

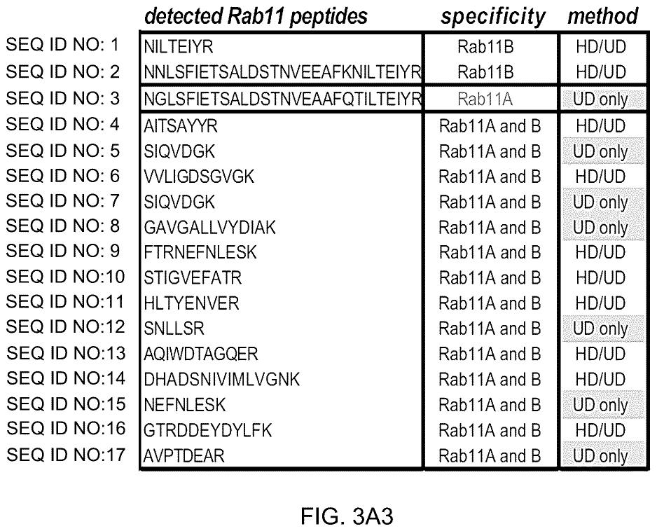

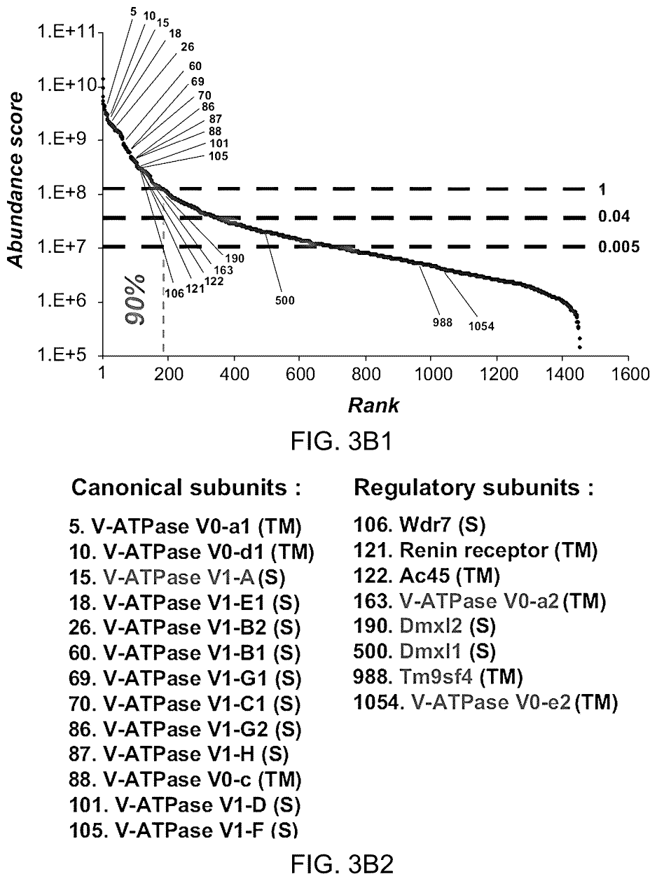

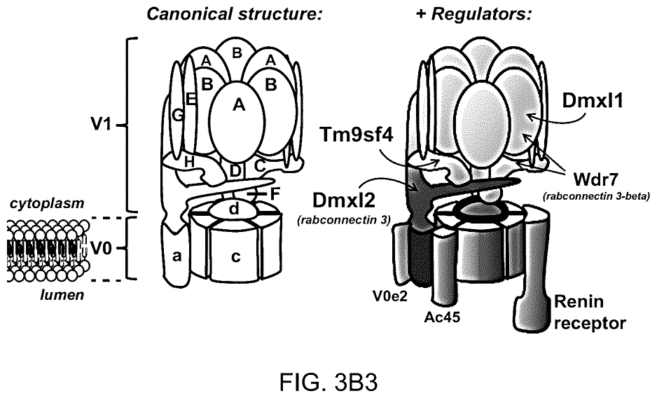

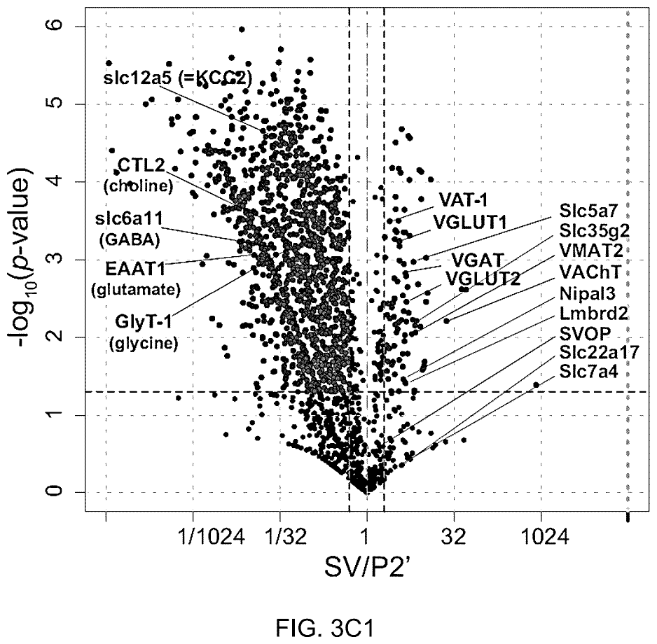

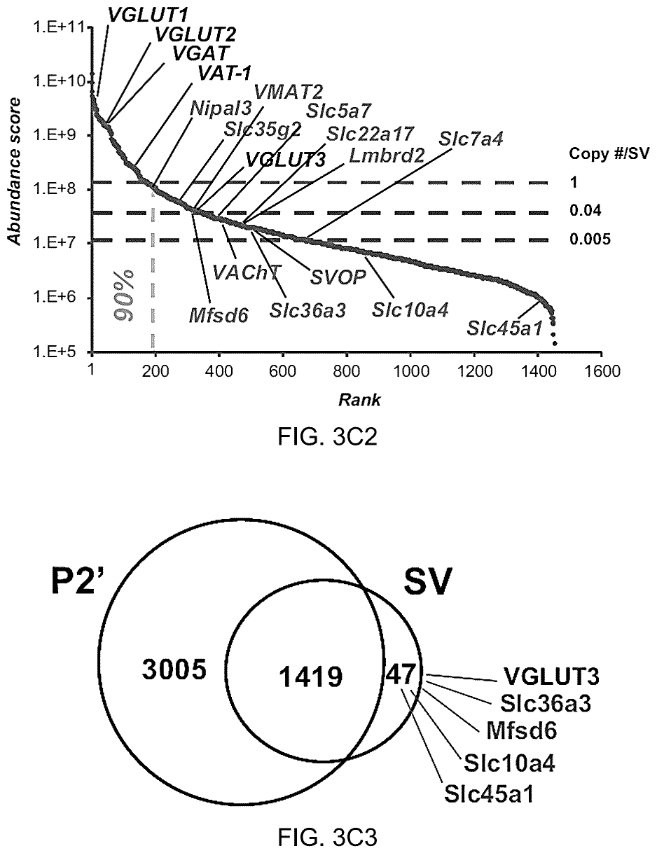

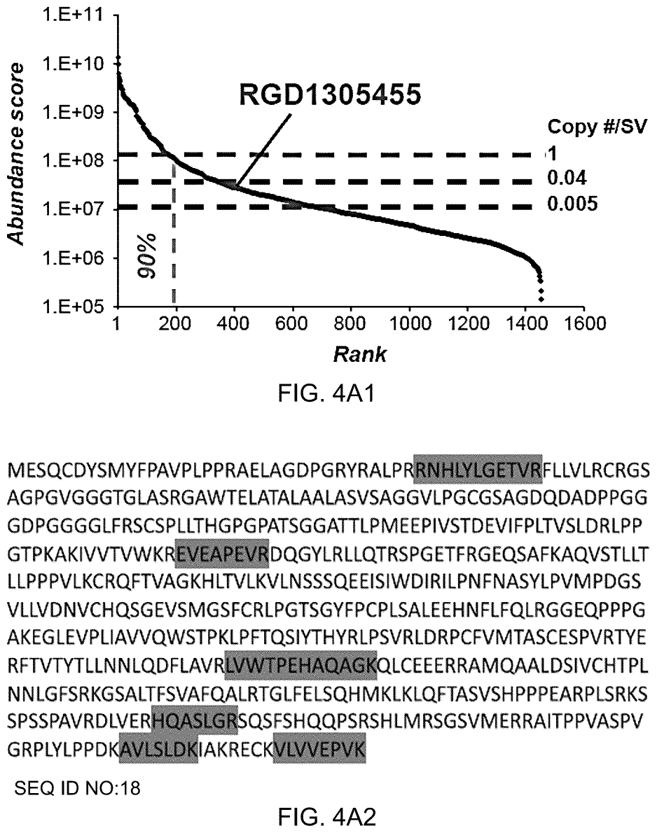

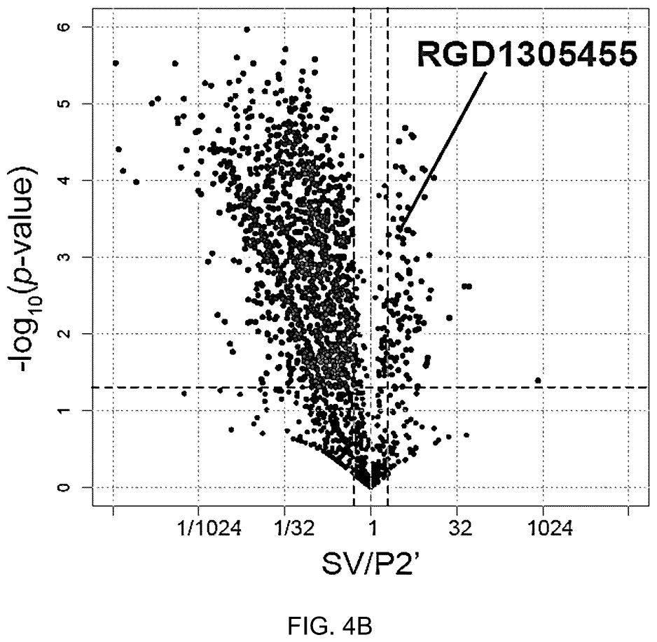

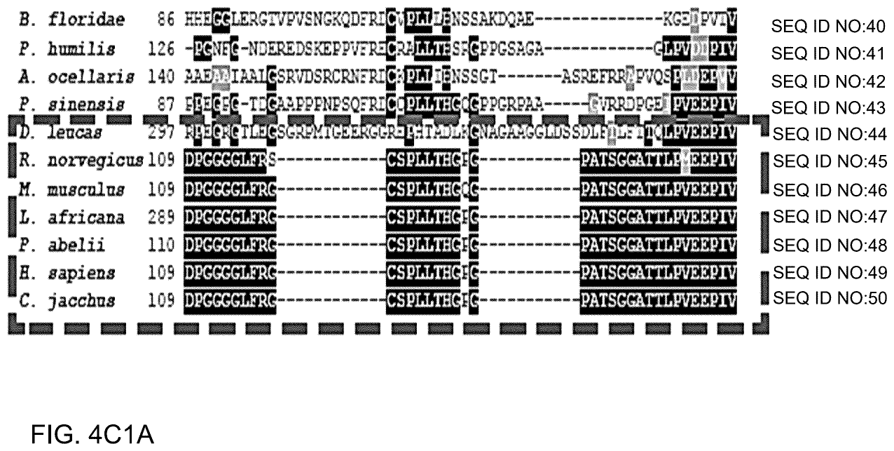

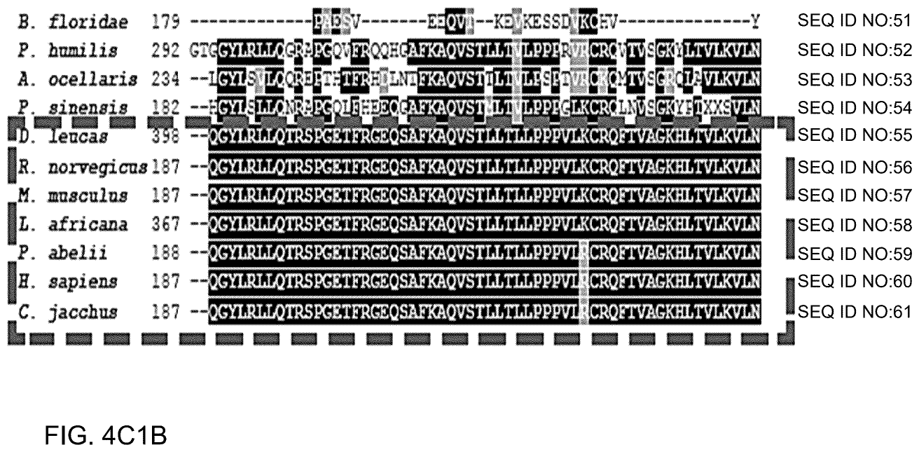

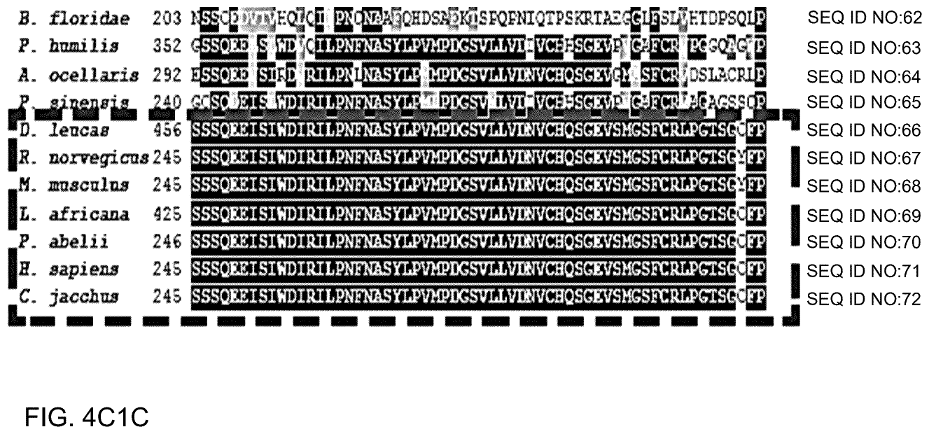

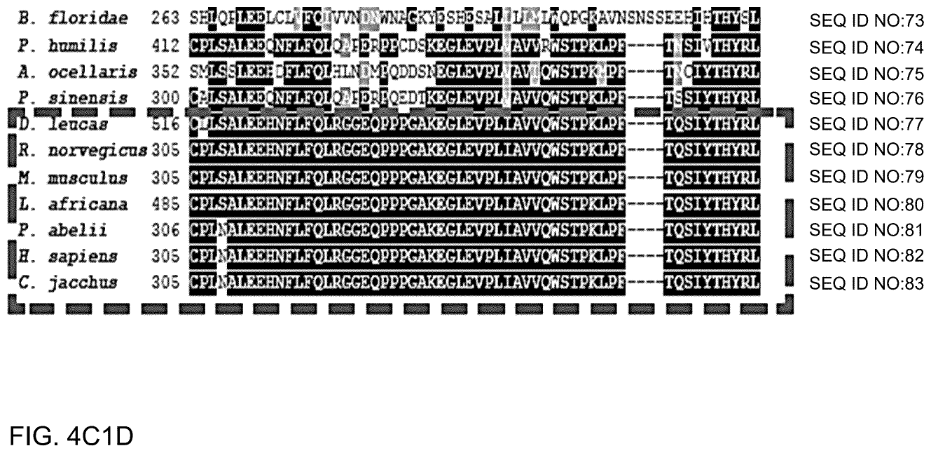

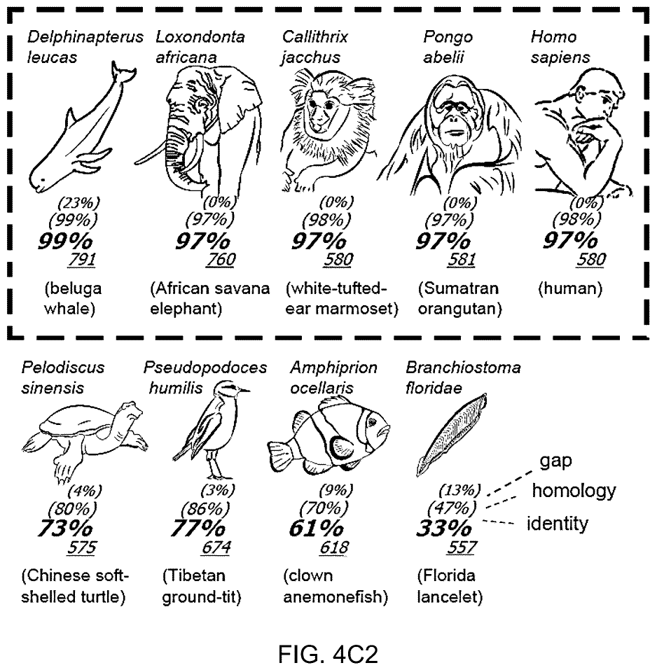

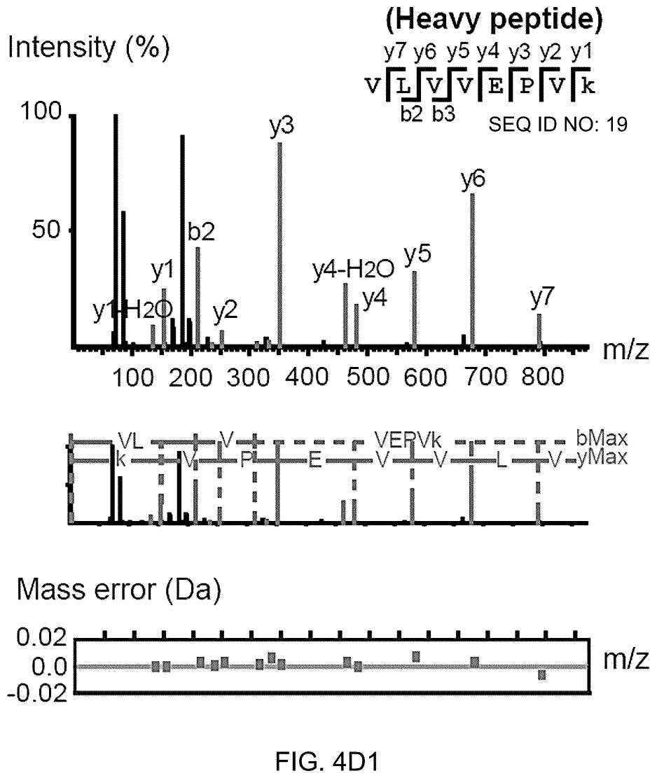

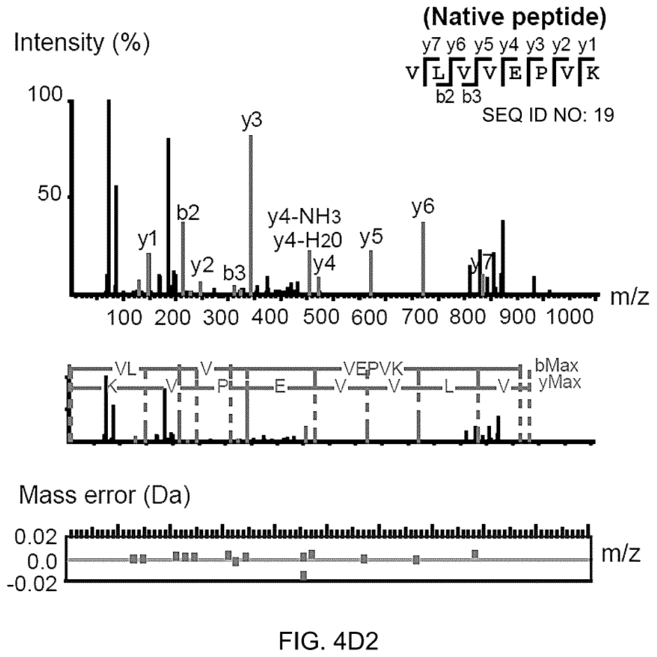

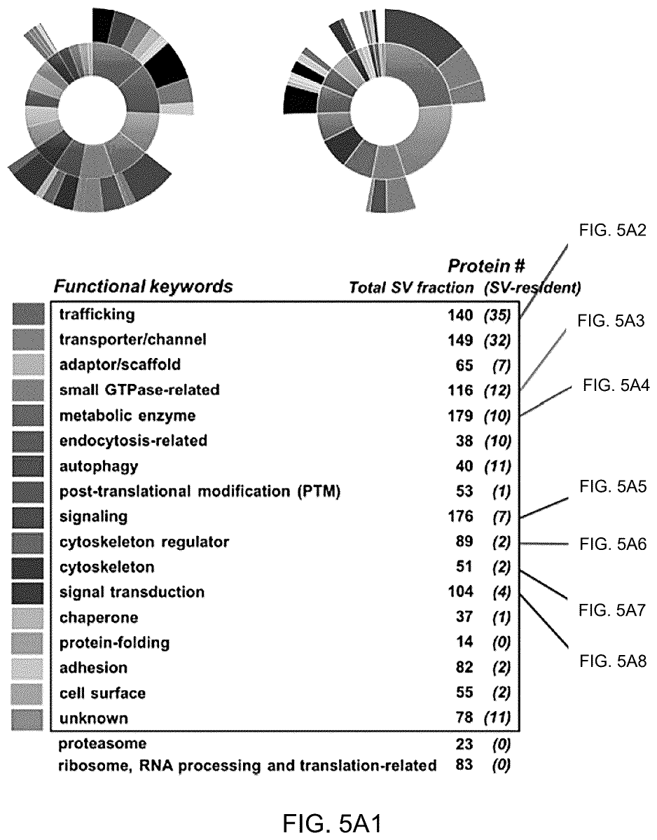

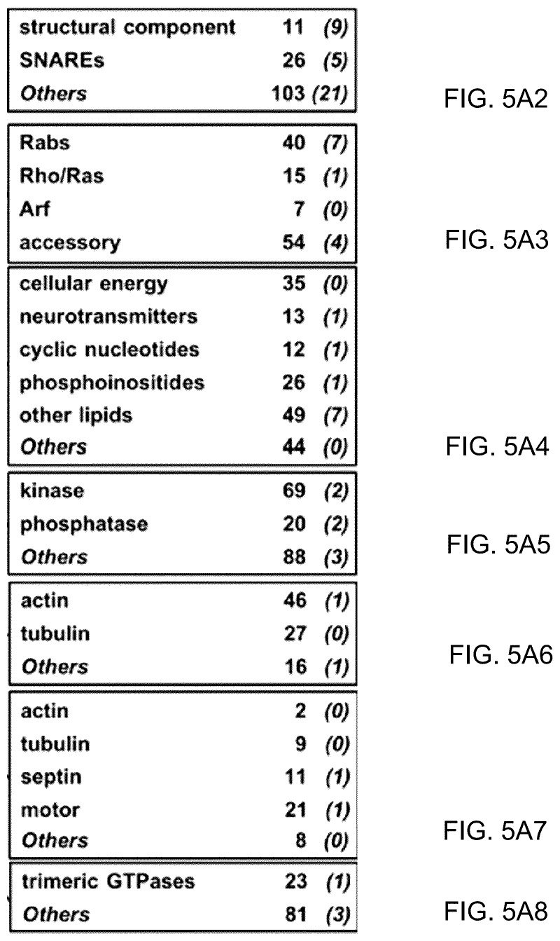

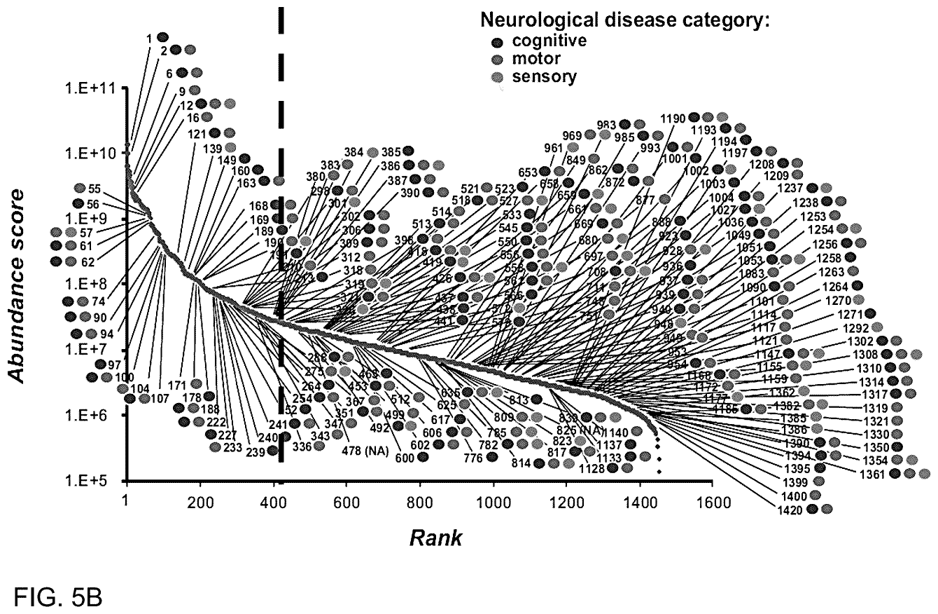

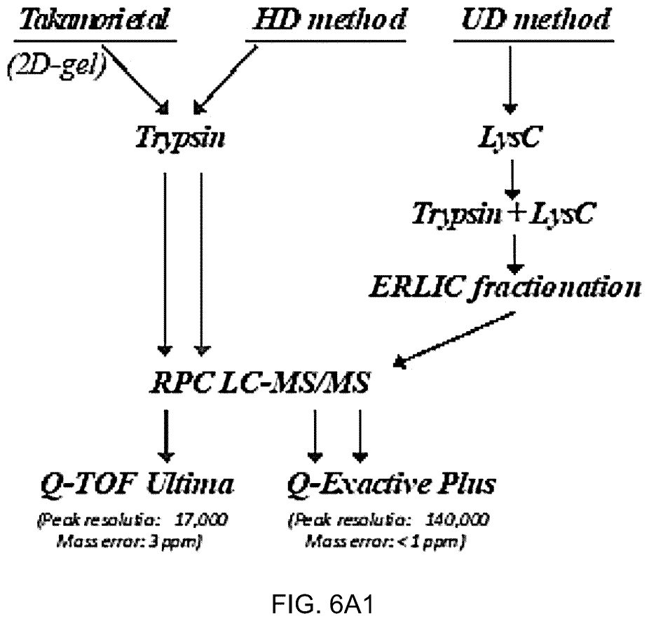

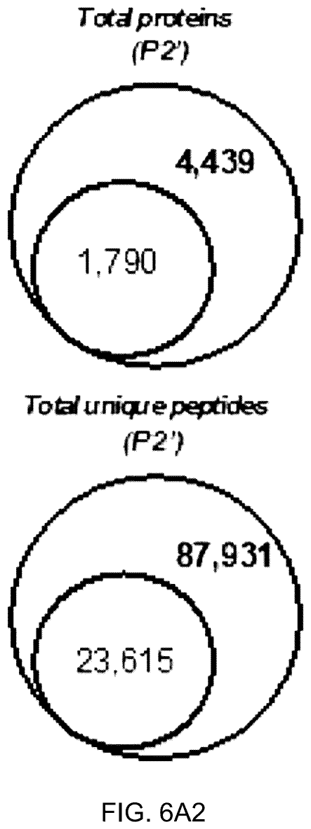

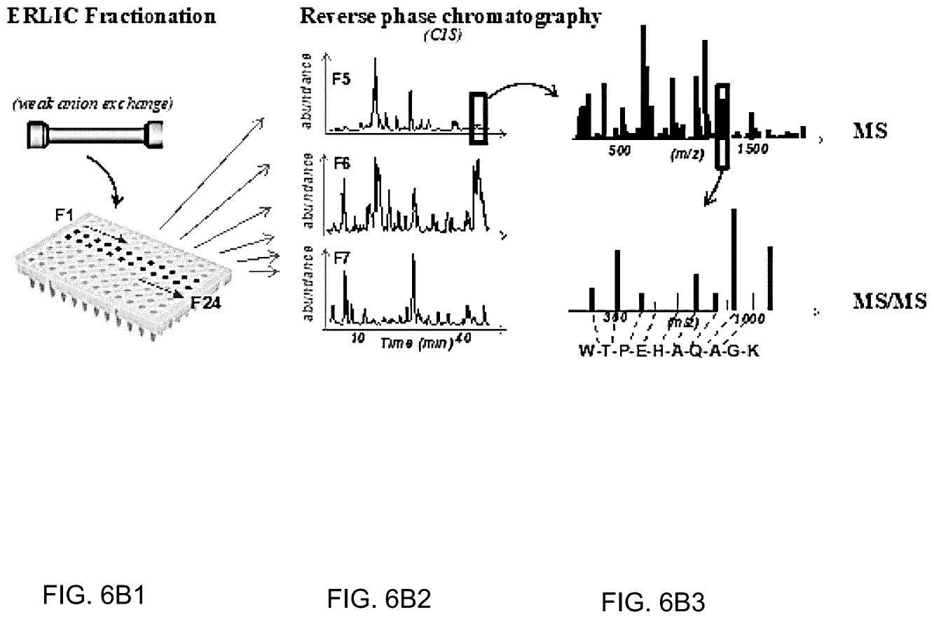

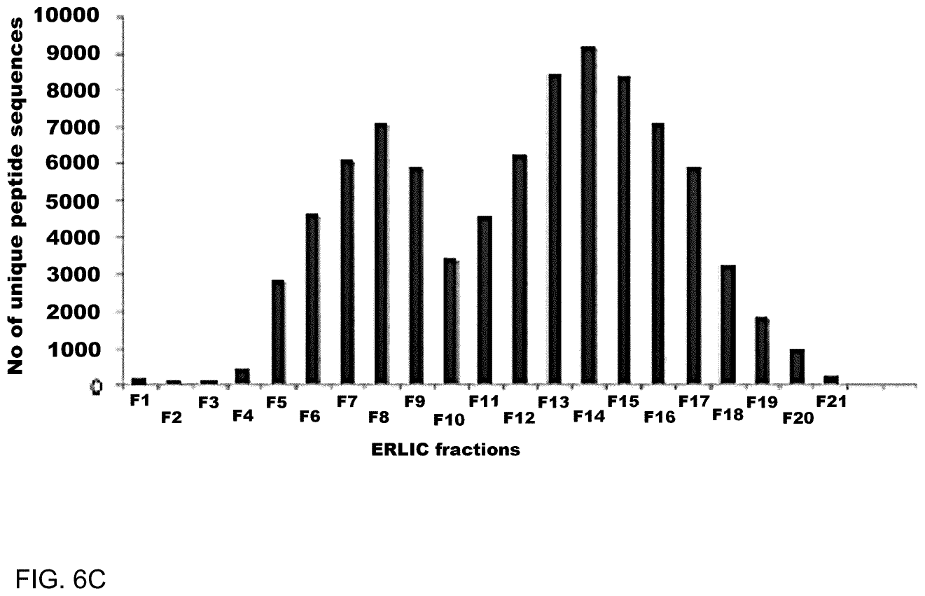

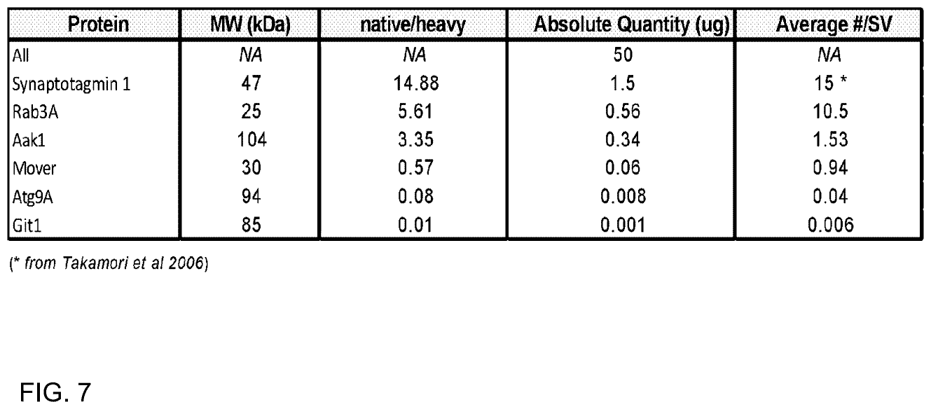

We next ranked the 1,466 proteins detected in the SV fraction by iBAQ abundance. We confirmed that previously reported canonical transmembrane proteins and lipid-anchored proteins were highly abundant. The 180 most abundant protein species accounted for 90% of the total protein mass of SVs, having iBAQs>1.2×10⁸ (1.2E8). The iBAQ of the remaining 1,286 proteins ranged from E5 to E8. Previously, copy numbers per SV were estimated for abundant SV proteins to construct an ‘average SV’ model (2, 6). Using isotope-labeled peptides, we extended the copy number estimate to newly detected SV proteins (See FIG. 7). As a calibration standard, we utilized the previously determined copy number of Syt1: 15 (2). The copy number estimated by this method for Rab3A was 10.5, which nearly coincided with the copy number of 10 previously determined by immunoblotting (2), confirming the accuracy of this method. These analyses indicated that copy numbers of many SV proteins are below 1, suggesting that they are present only in subpopulations of SVs or only transiently interact with SVs.

The “hidden SV proteome” uncovered by the UD proteomic method To reveal the hidden SV proteome, we tabulated an SV protein inventory detected by UD proteomics with annotations, including comparisons with those by Takamori et al (2006). This inventory allows to extract novel insights into SV structure and function using various filters, such as gene family names, abundance rank, and molecular, structural, or functional categories. The first example selected from the inventory is the Rab GTPases, which function in vesicle transport to specific subcellular organelles and membranes (20). They are evolutionally conserved, displaying 75-95% amino acid sequence identity. Such high homology has hampered proteomic detection, but using UD proteomics, we detected and quantified 40 Rabs in the SV fraction, of which 8 are newly reported. Of 32 Rabs previously documented (2), abundance was quantified for only 18 using western blot analysis (21). We found a majority of high-abundance Rabs (25/40) significantly enriched in the SV fraction. Among them, Rab11A and Rab11B are highly homologous, with 91% amino acid sequence identity (FIG. 3A). Despite such similarity, they reportedly function in opposing endosomal sorting routes (22). We found 14 unique peptides common to both Rab11A and B; however, only UD proteomics could detect a Rab11A signature in the C-terminal hypervariable region. Thus, UD proteomics can reveal highly homologous, but functionally distinct proteins.

The second example is the vacuolar-type H⁺-ATPase (V-ATPase) protein complex, which operates as an ATP-driven proton pump to energize SVs for neurotransmitter uptake. The V-ATPase complex is composed of a cytoplasmic domain ‘V1’ comprising 8 subunits (A-H), and a transmembrane domain ‘V₀’ assembled from 4 subunits (a, c, d, e) (23) (FIG. 3B). Previous proteomic studies estimated the copy number of V-ATPase as ~1-2/SV, but the complete set of V-ATPase proteins remains unidentified (2, 4, 17). Intriguingly, using UD proteomics, we identified all components of V-ATPase complex, most of which were found in the high-abundance range of the SV proteome (FIG. 3B). Furthermore, V-ATPase accessory proteins Wdr7 and renin receptor (atp6ap2), and previously hidden Dmxl1 and Dmxl2, were all identified (24) (FIG. 3B). These low-abundance accessory proteins, in which only renin receptors are categorized as SV-resident, may regulate V-ATPase complex functions in a restricted subset of SVs. Thus, the UD method can reveal full sets of subunits comprising large protein complexes.

The third example is SV-resident transporter proteins. Solute carrier (‘slc’) transporters are transmembrane proteins that control movements of soluble molecules across cellular membranes. To date, more than 400 slc genes have been identified in mammals, of which ~40% remain uncharacterized with respect to their expression profiles and functions (25). Our UD analysis detected slc transporters both in SV-resident and SV-visitor repertoires (FIG. 3C). The latter may include transporters partially internalized from plasma membrane into SVs during endocytosis. SV-resident transporters include VGLUT1 (slc17a7) and VGLUT2 (slc17a6) responsible for glutamate uptake, and VGAT (slc32a1) for GABA and glycine uptake, all of which define the molecular identities of the major SV populations in the brain (26), and which occur at high abundance in the SV proteome (FIG. 3C). UD proteomics also detected lower abundance SV-resident transporters that were missing in previous SV proteomic studies. These include VMAT2 (slc18a2; (27)), ChT1 (slc5a7; (28)), VAChT (29), involved in uptake of monoamines or ACh into SV subpopulations and SVOP of unknown substrate (atypical slc subfamily, (30)). In addition to these well-known transporters, UD analyses identified 9 new SV-resident transporters (FIG. 3C), among which, slc10a4 reportedly transports bile acids into SVs to modulate dopamine activity (31). The remaining 8 transporters are orphan slcs of unknown function (Table S2). Thus, UD proteomics have unveiled and quantified hidden transporter proteins of both high and low abundance in the SV proteome, having ubiquitous or restricted presence in SV populations.

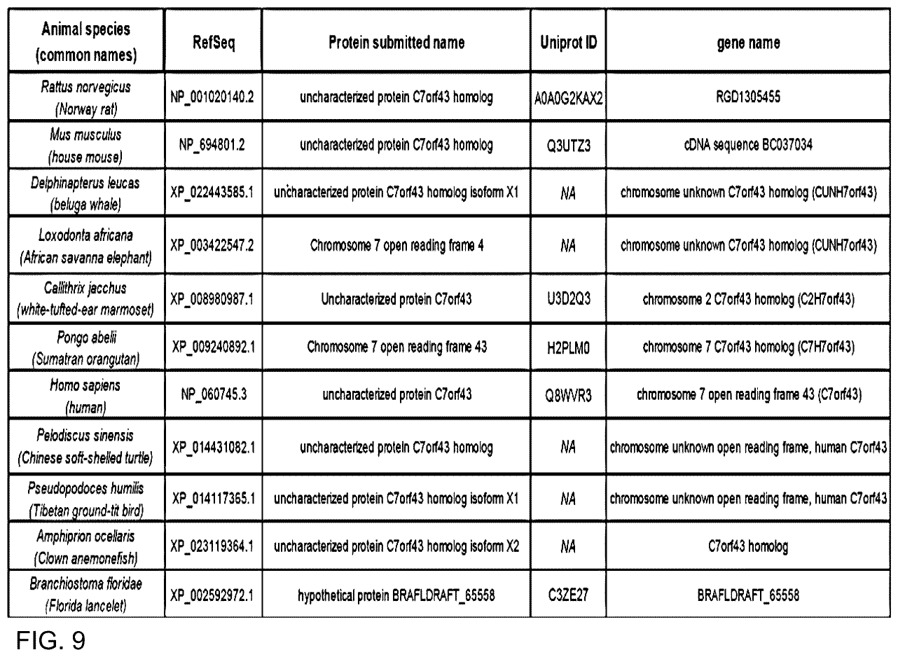

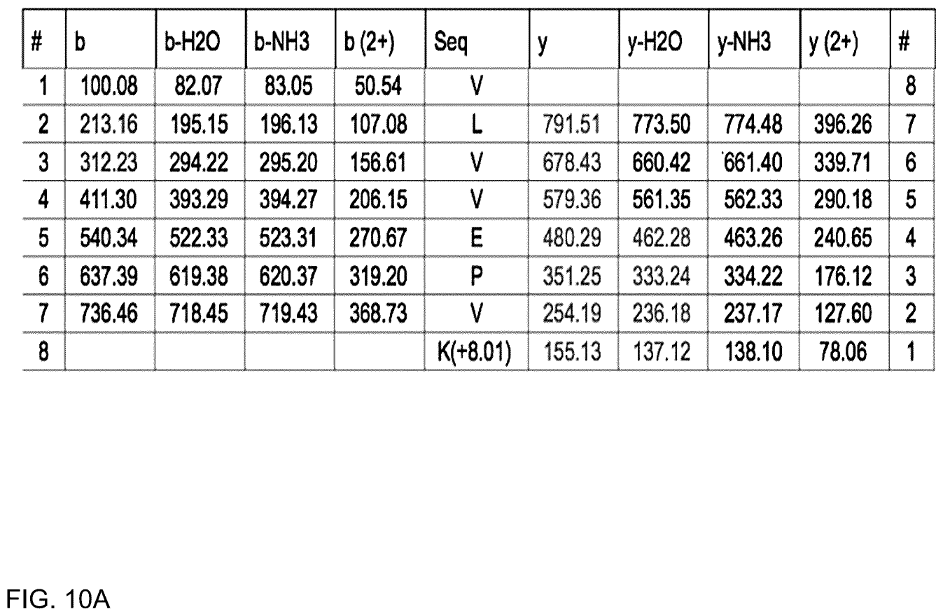

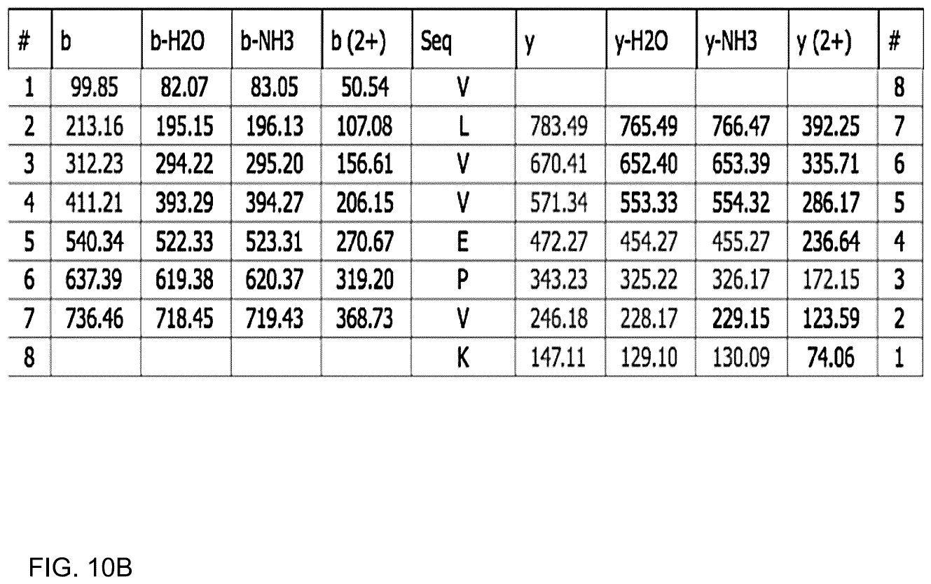

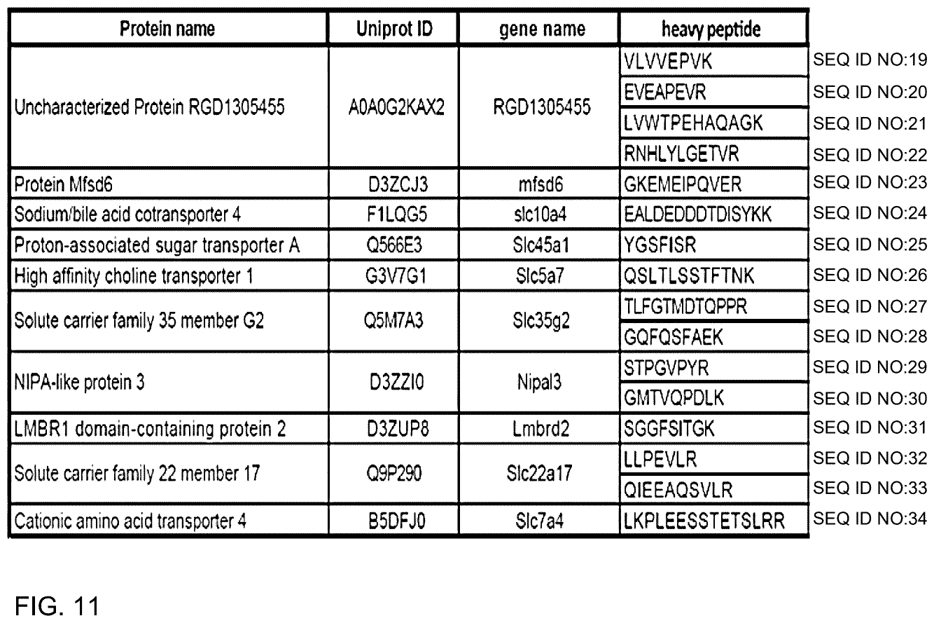

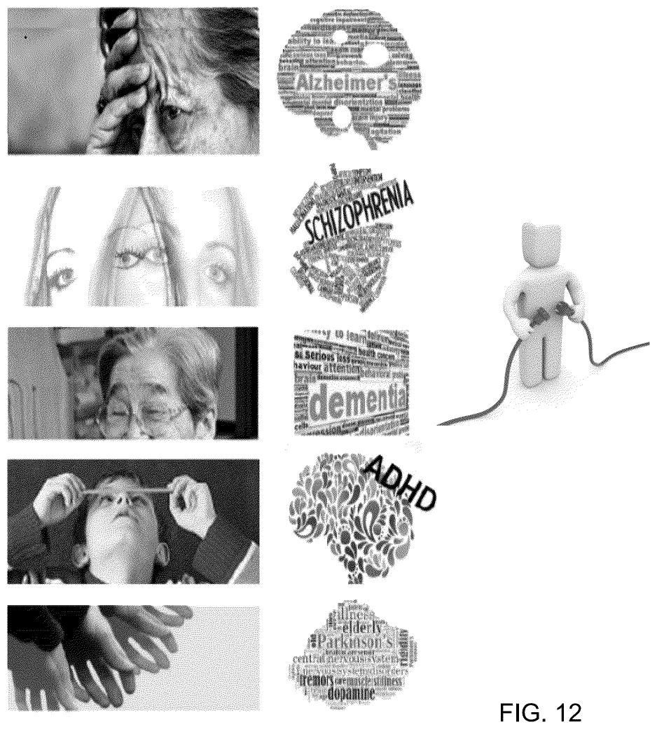

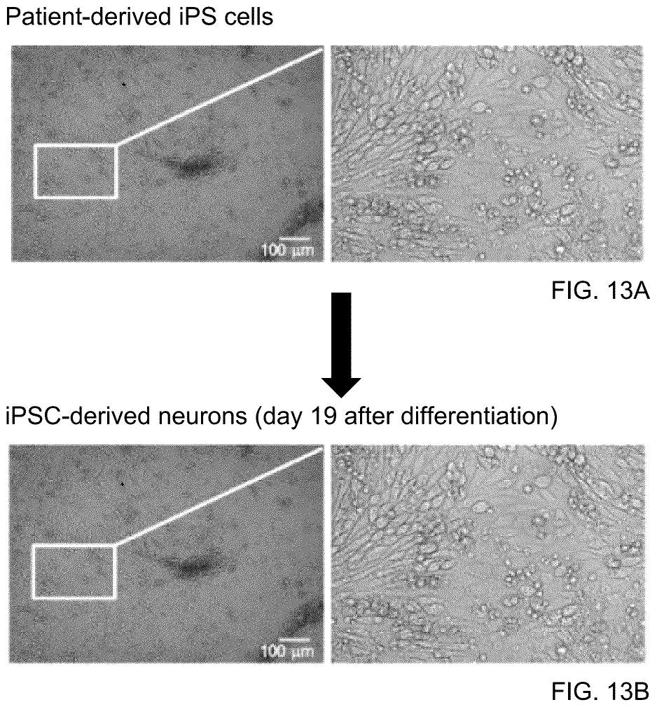

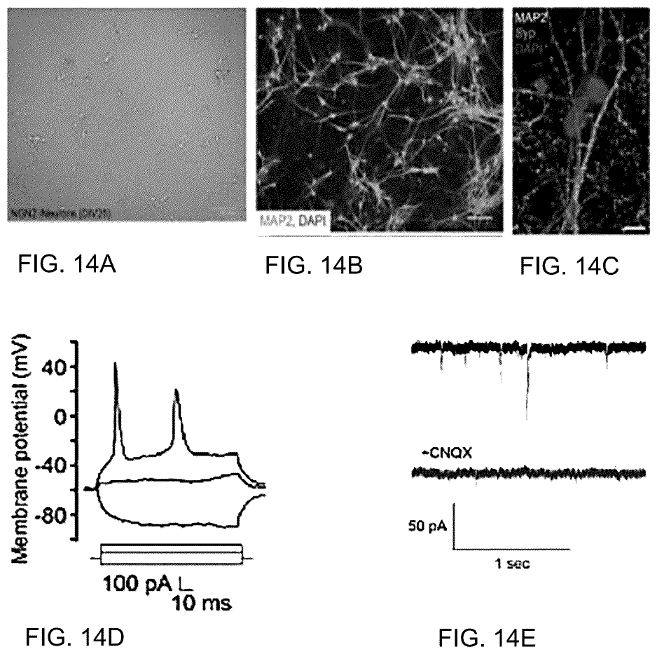

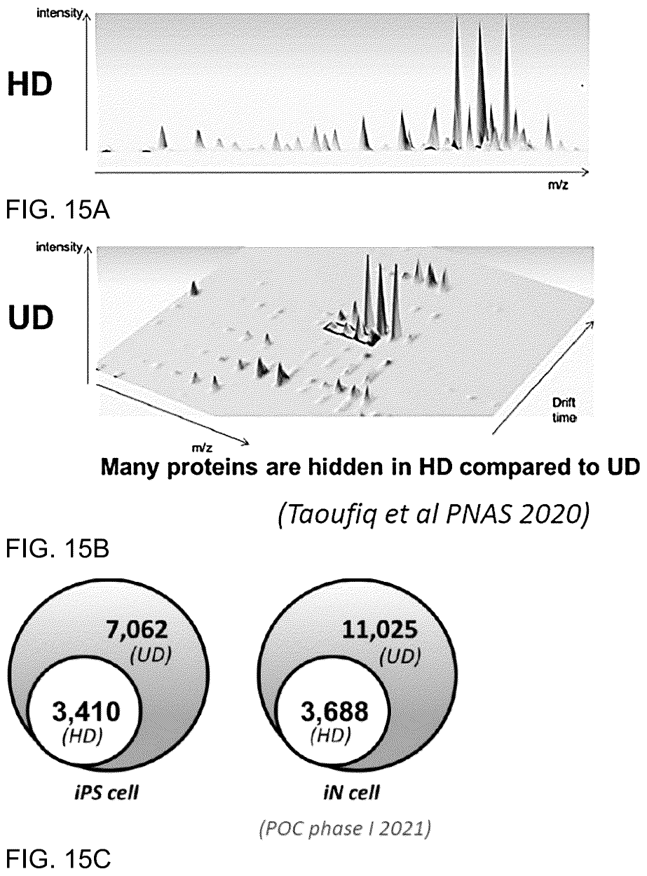

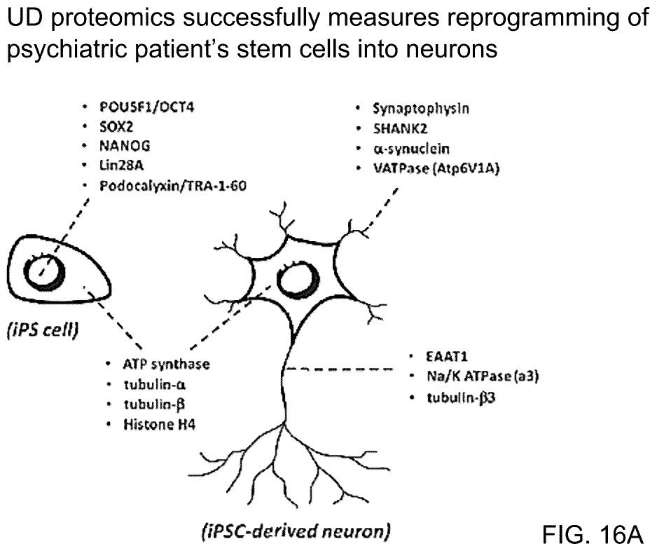

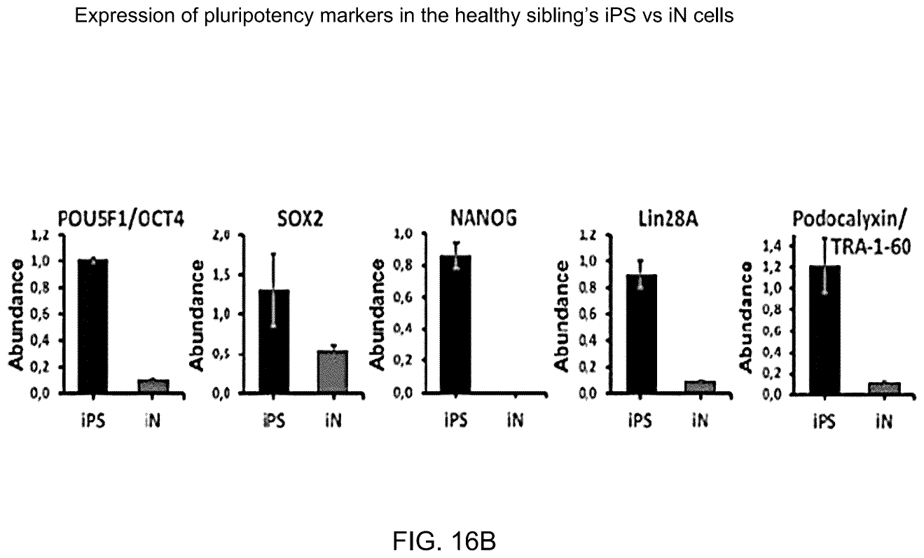

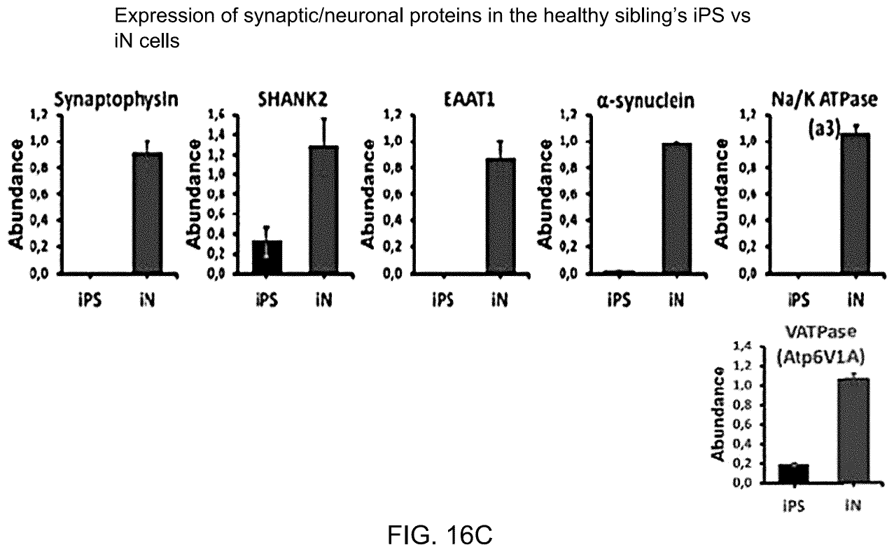

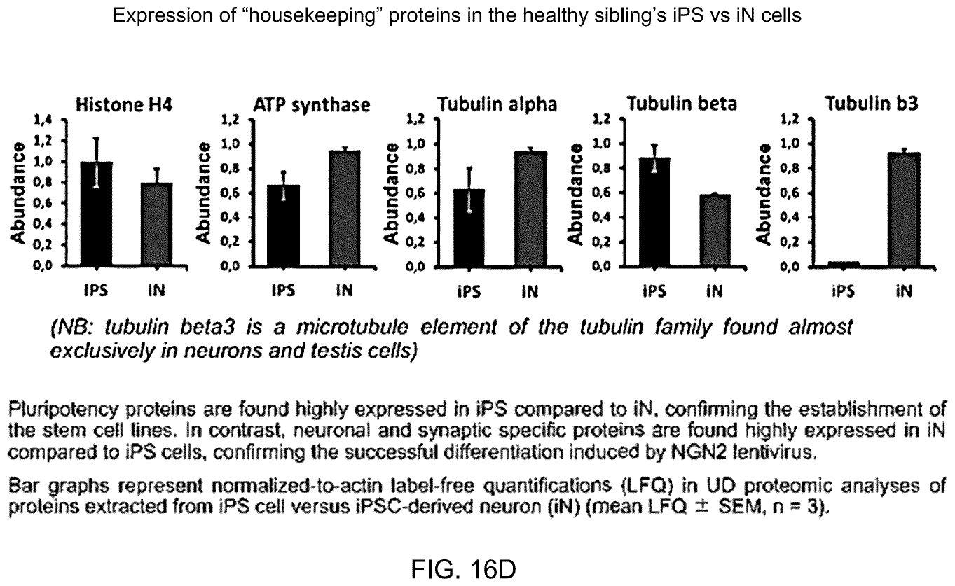

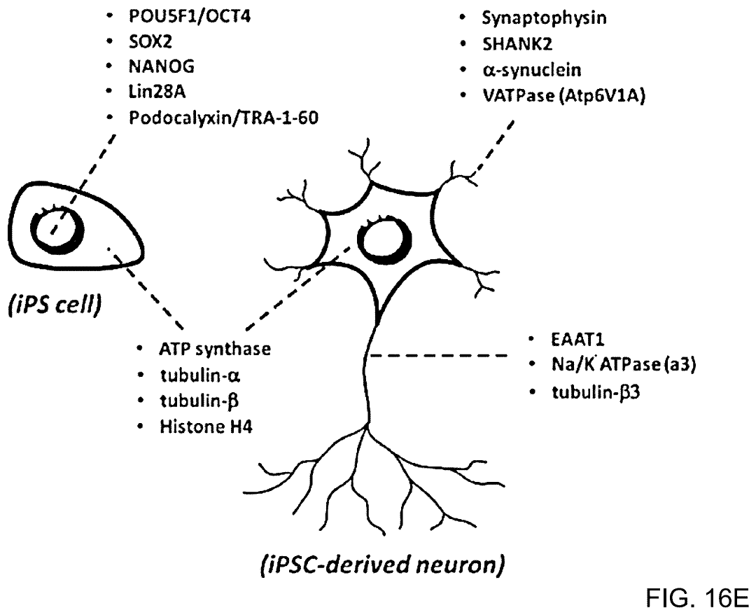

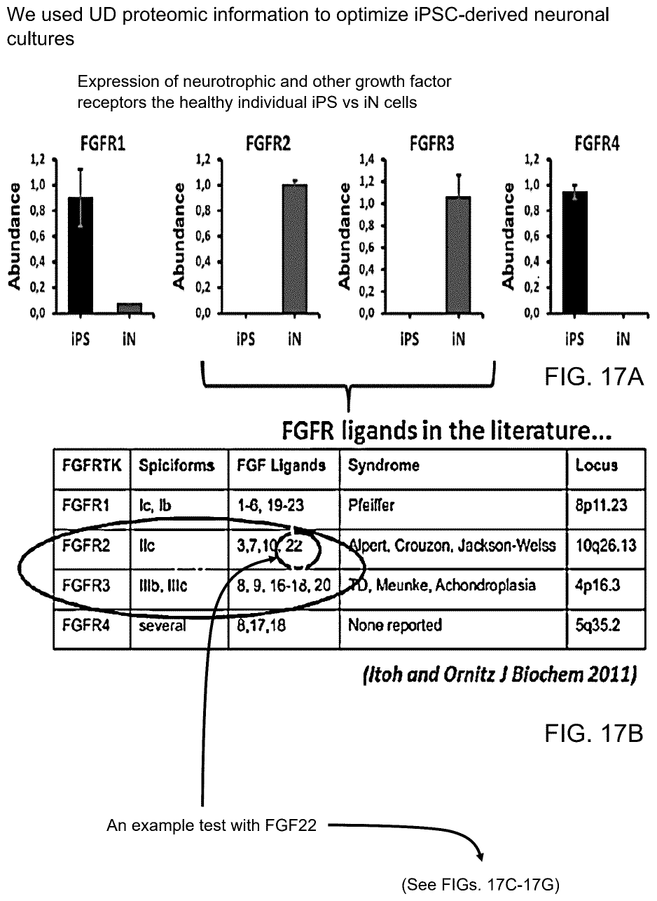

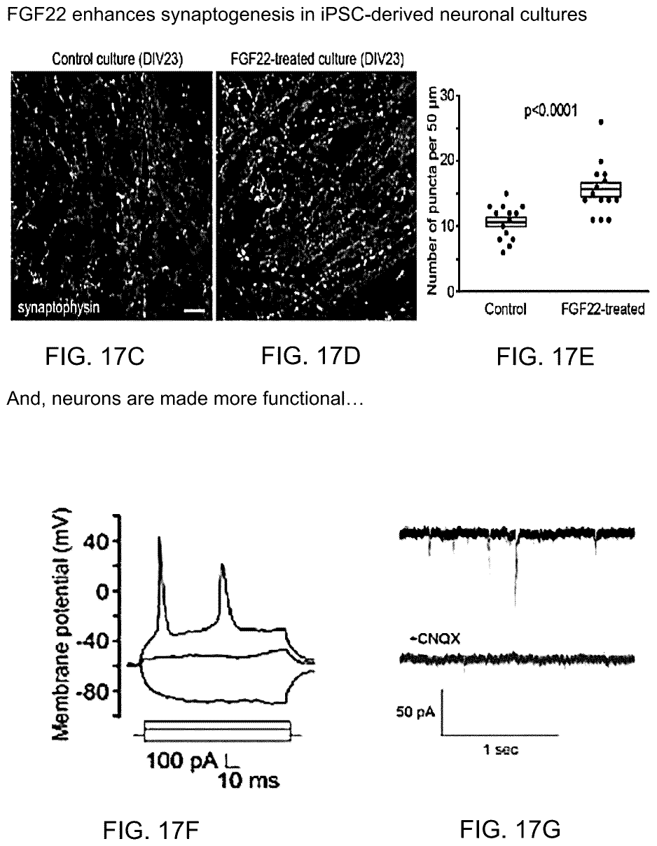

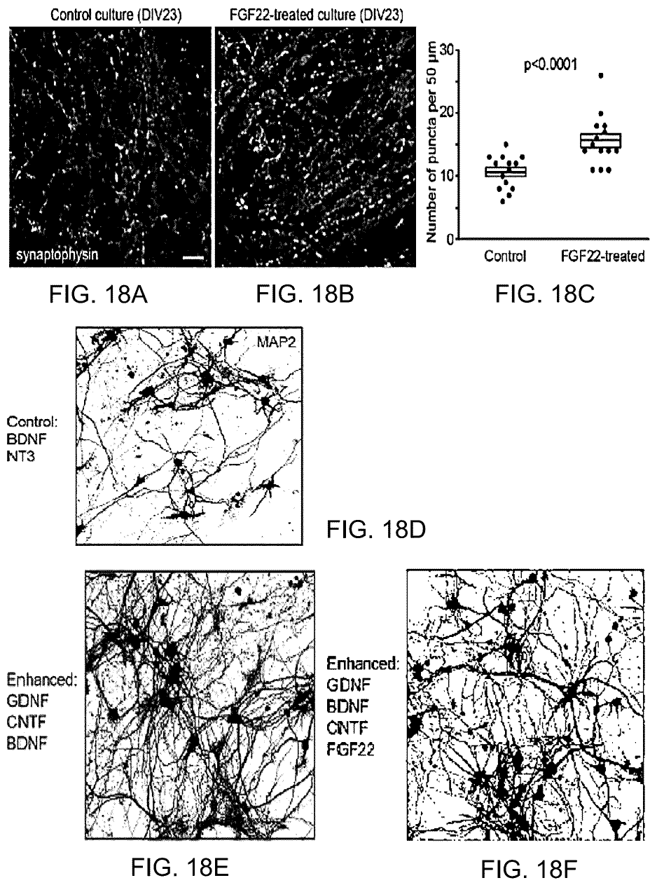

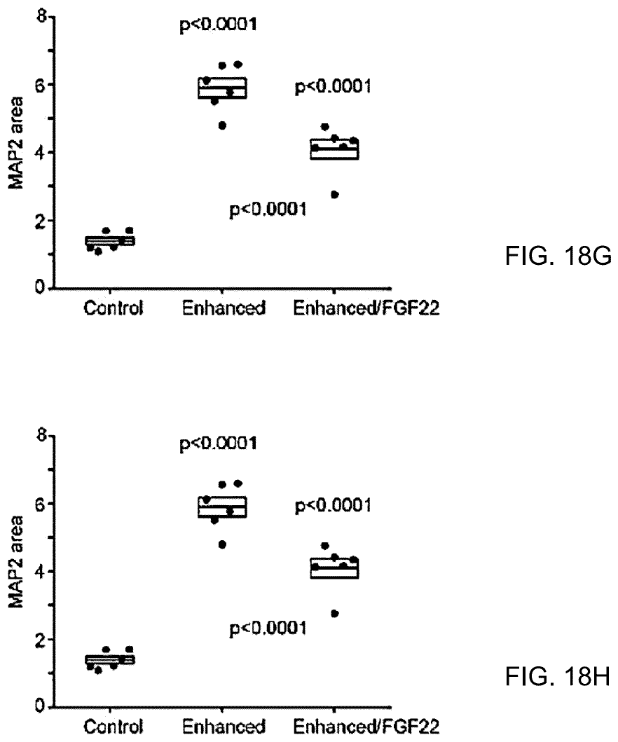

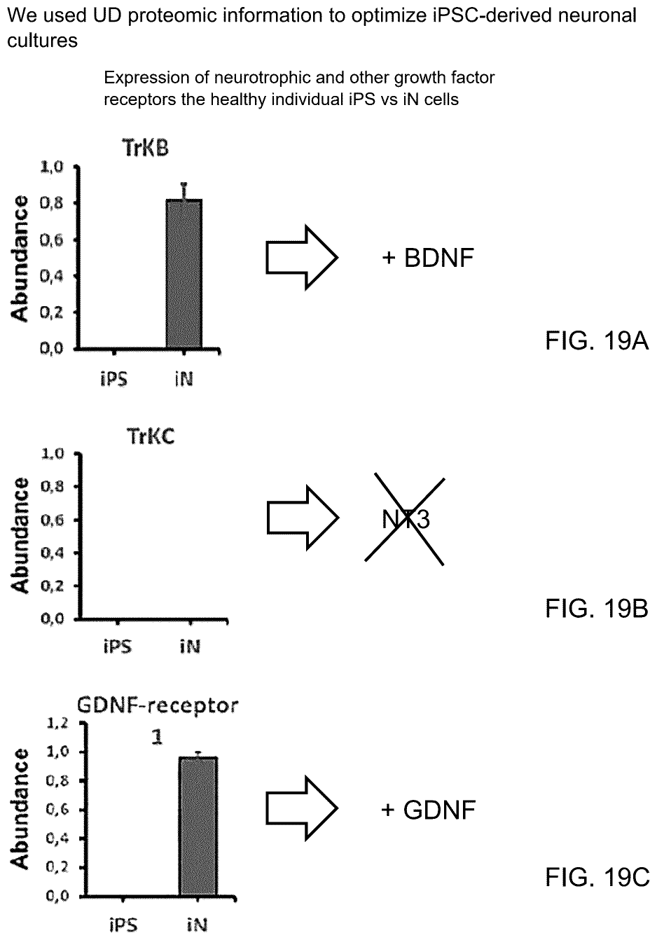

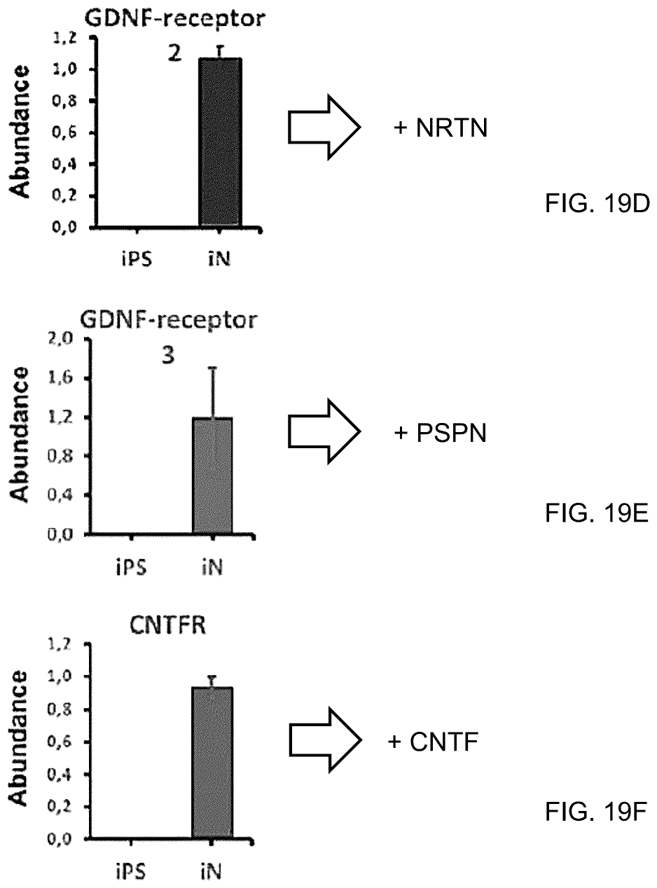

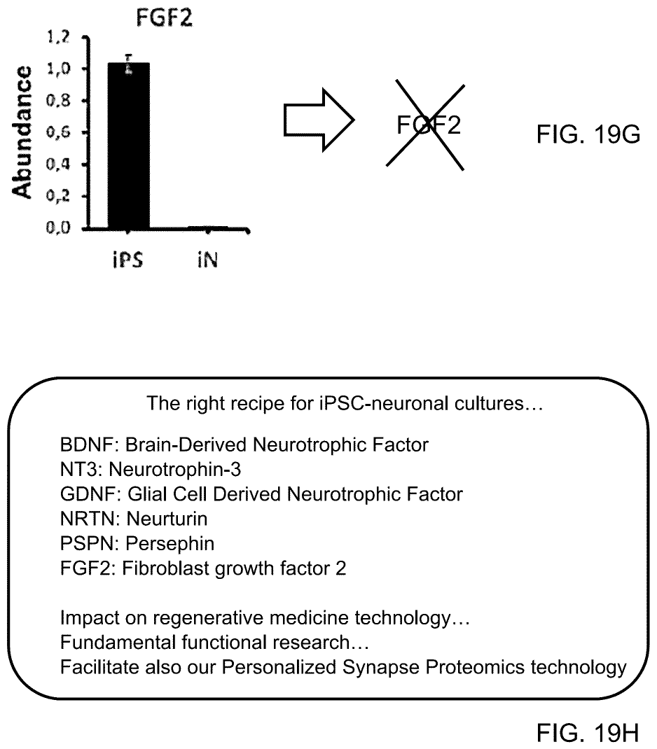

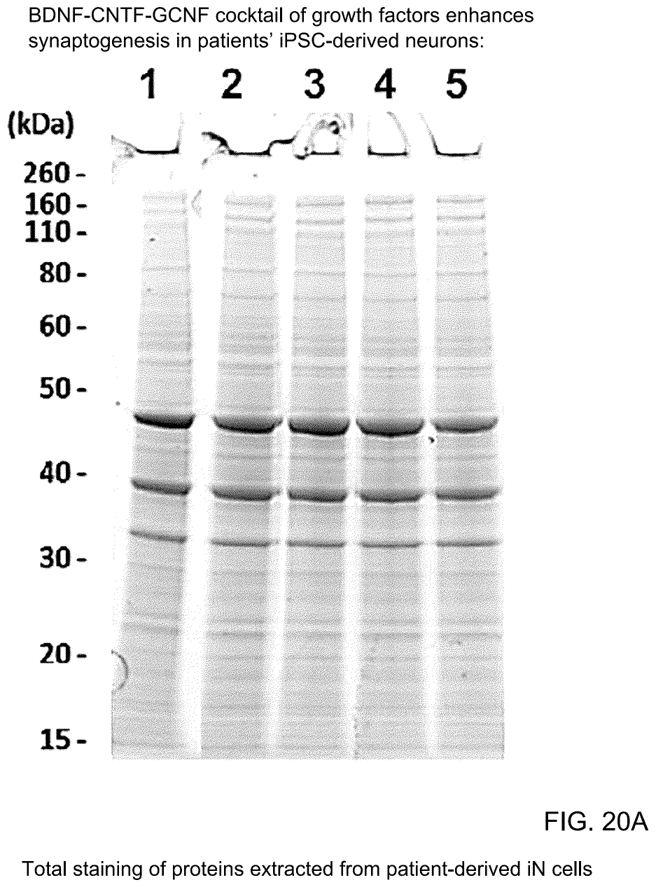

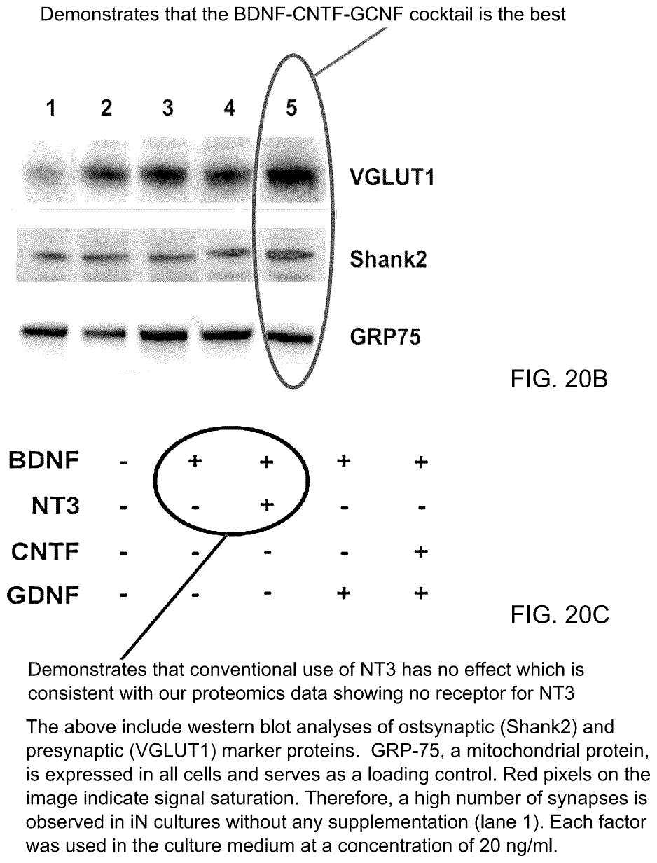

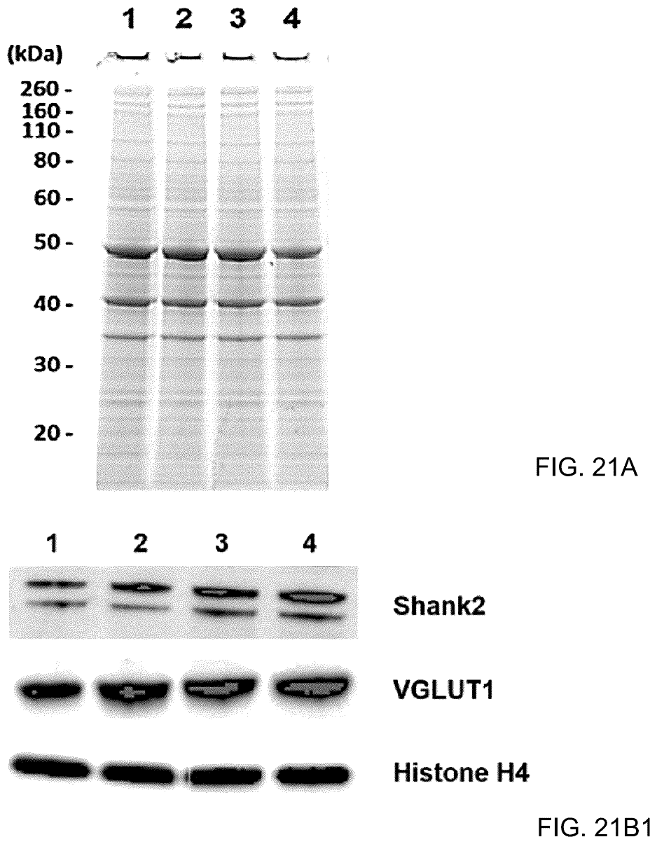

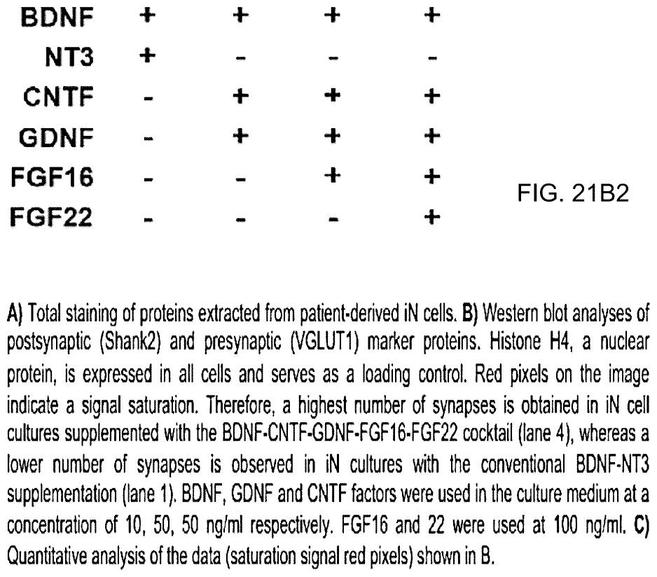

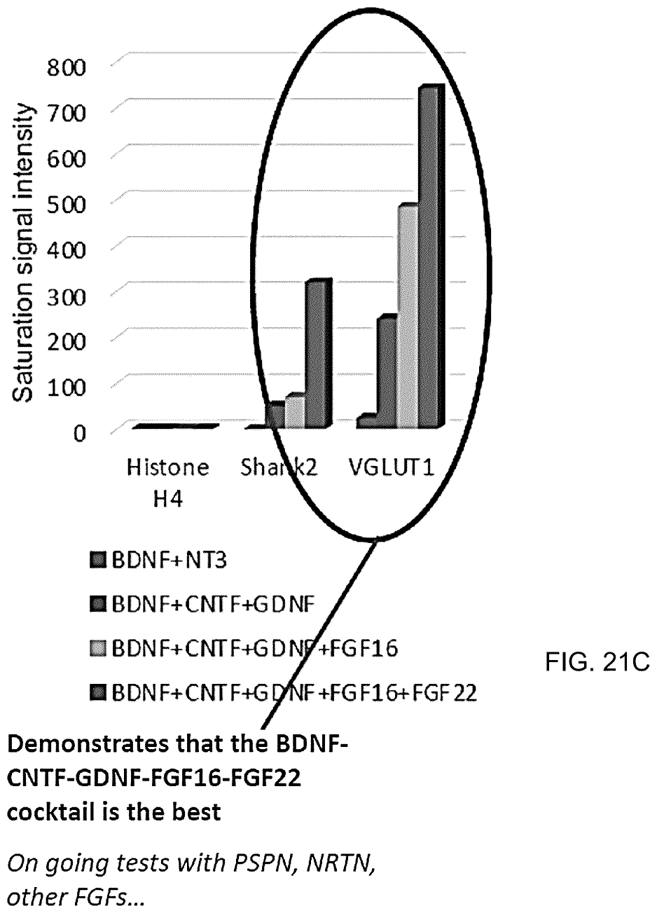

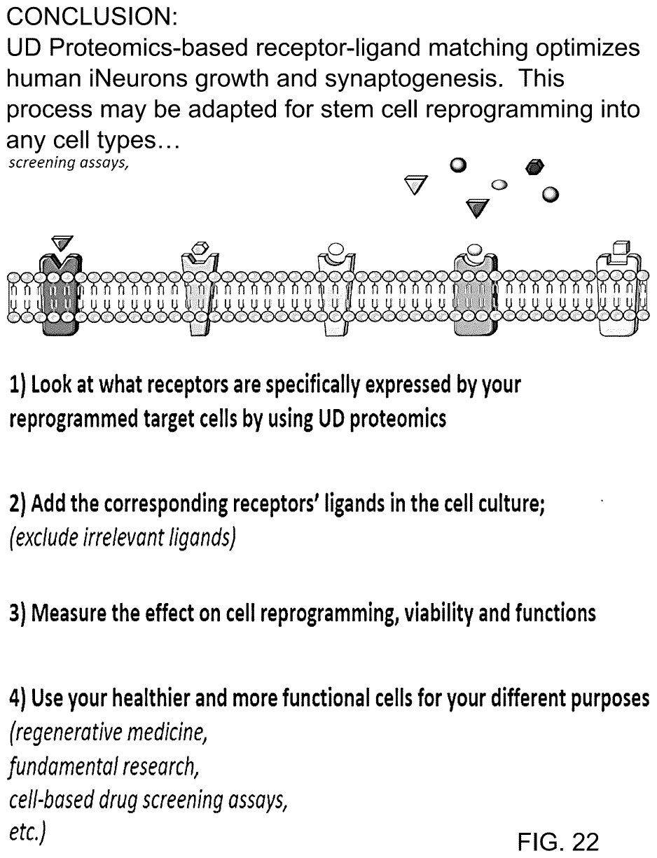

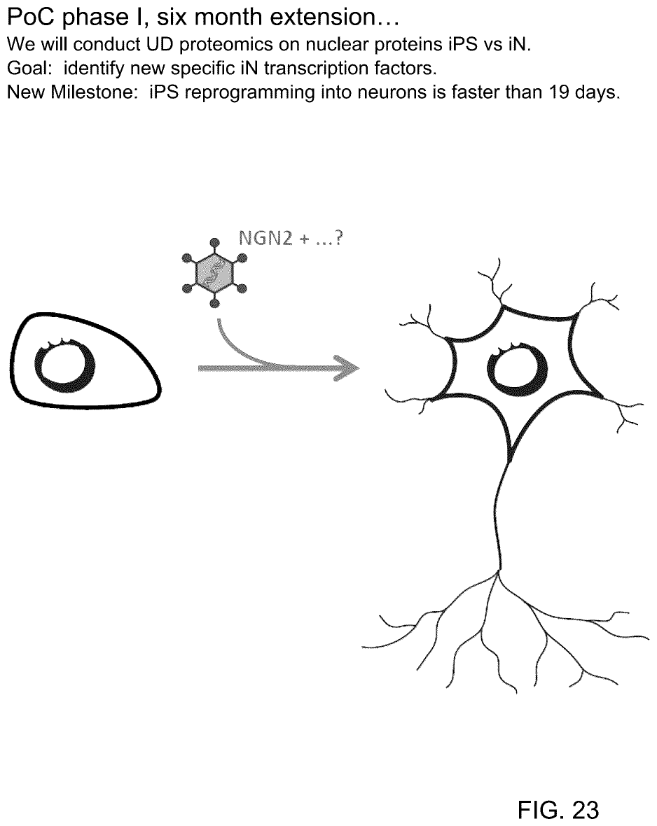

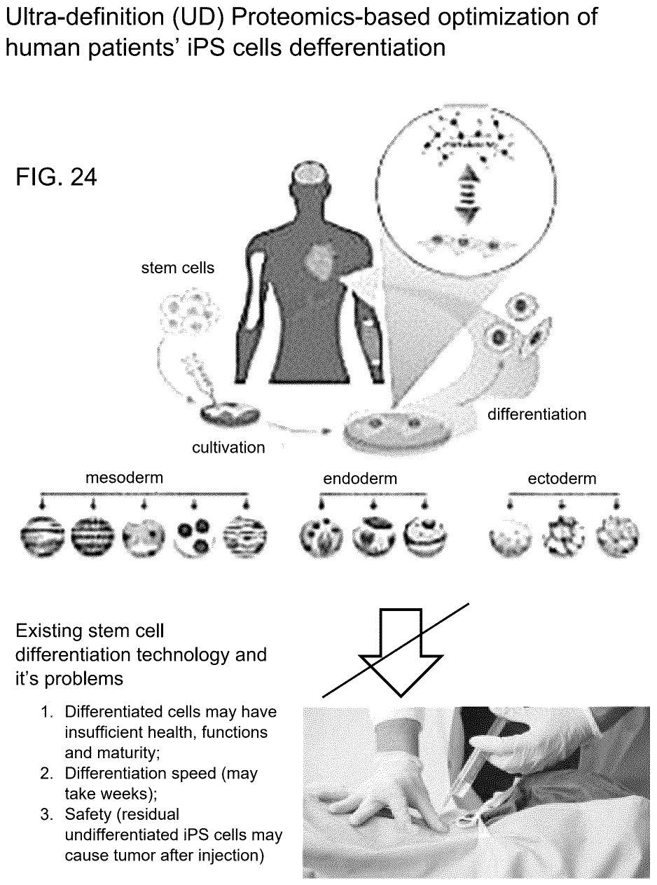

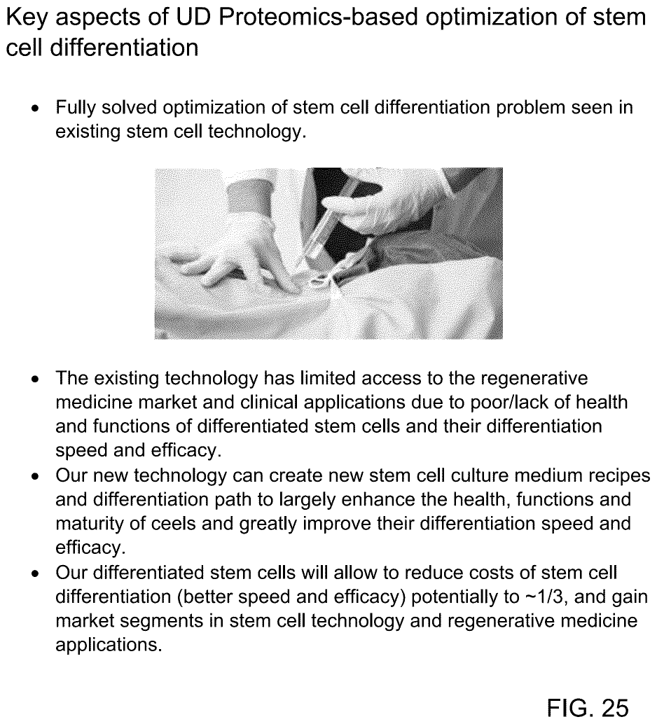

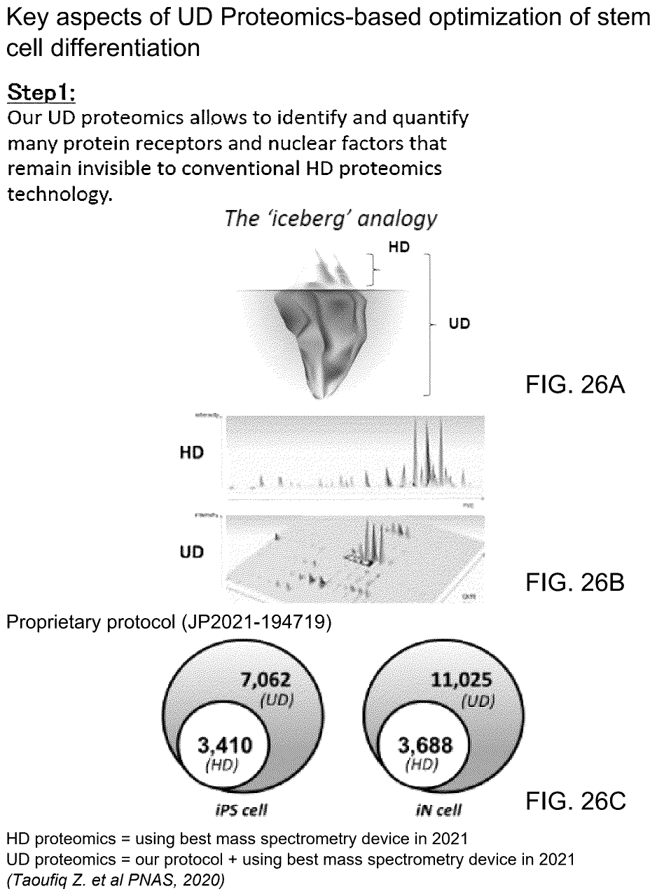

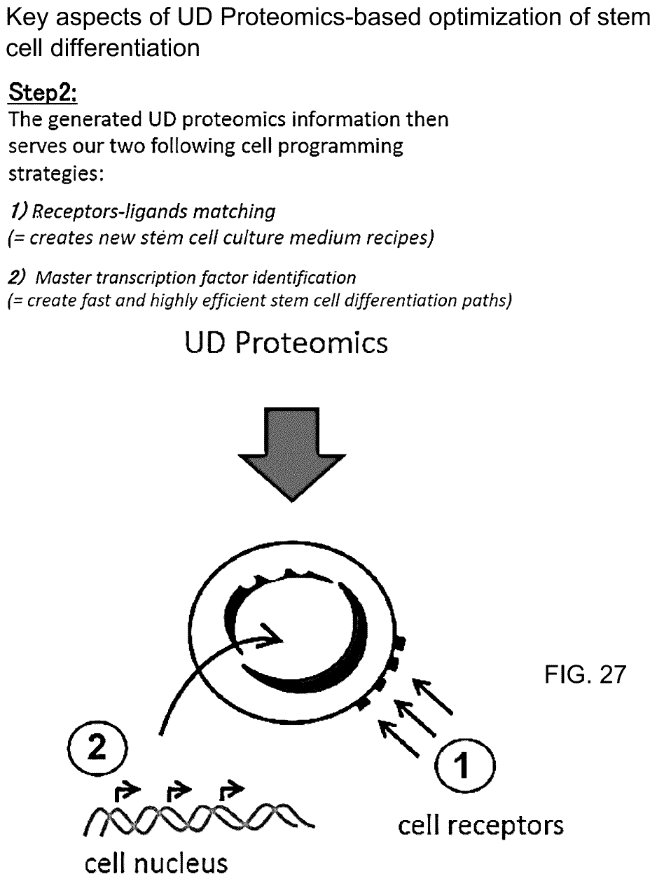

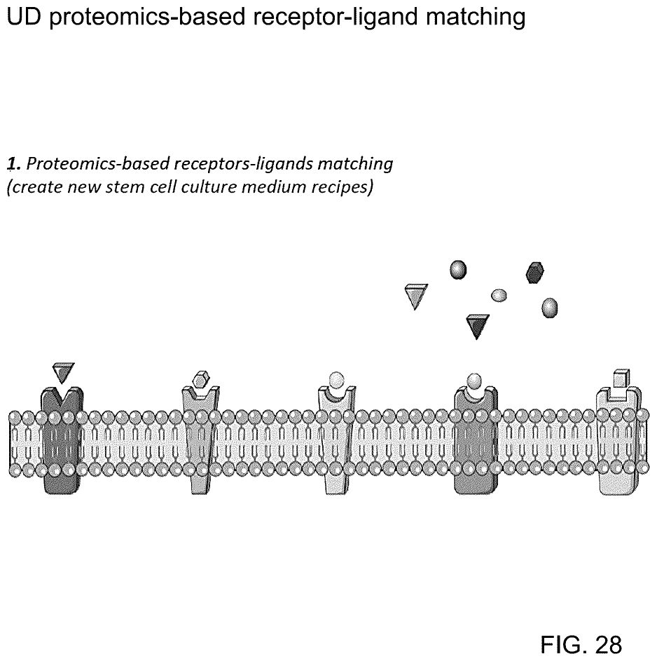

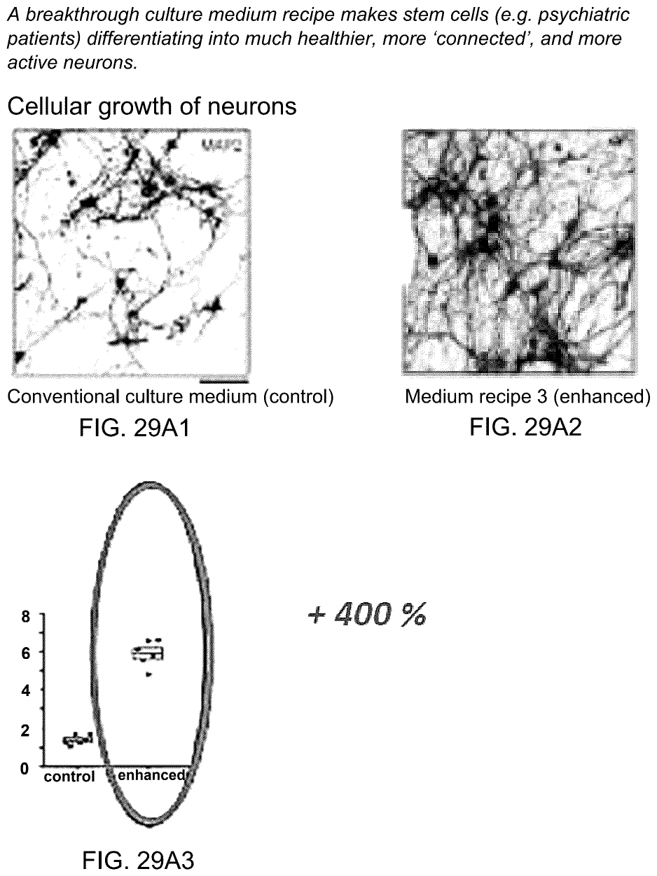

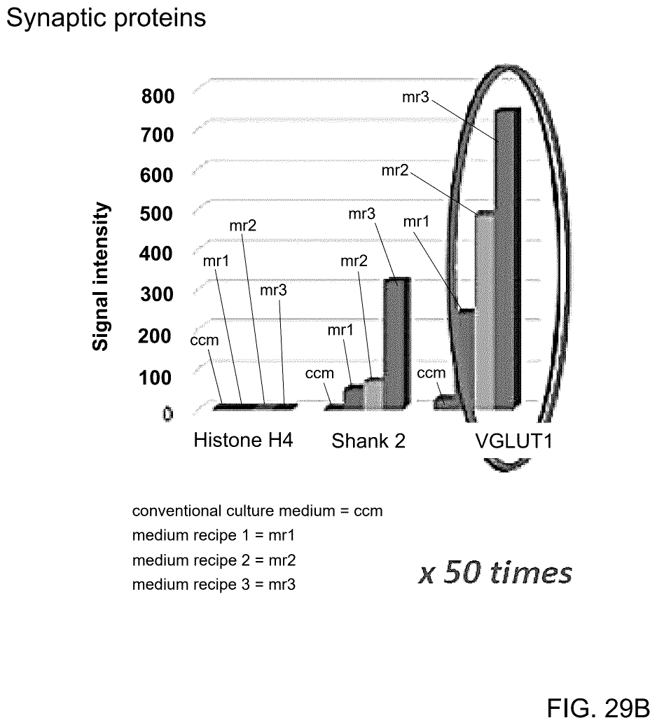

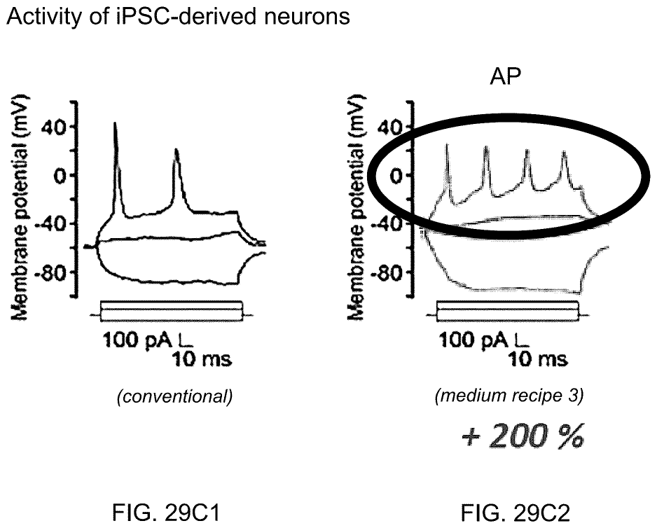

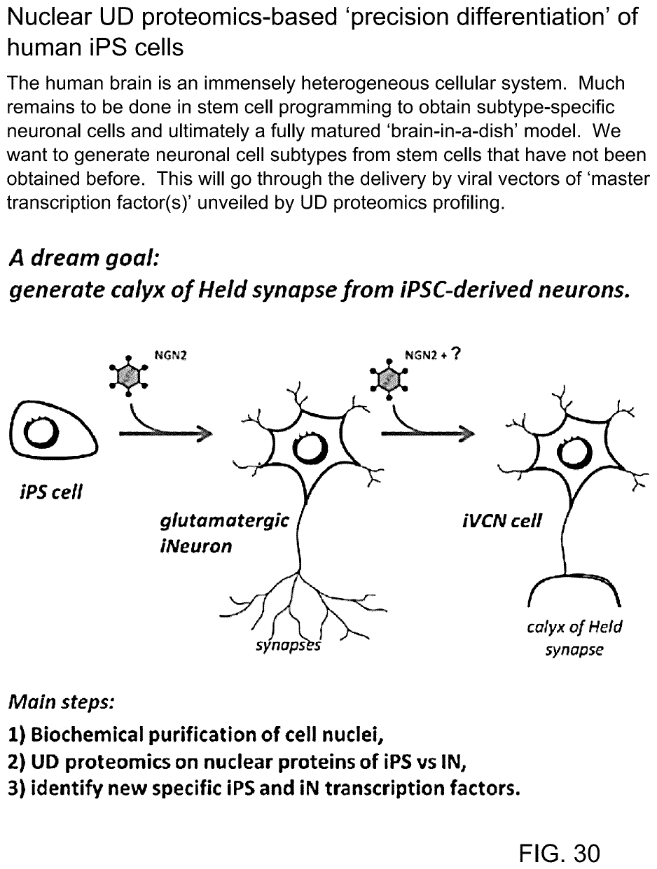

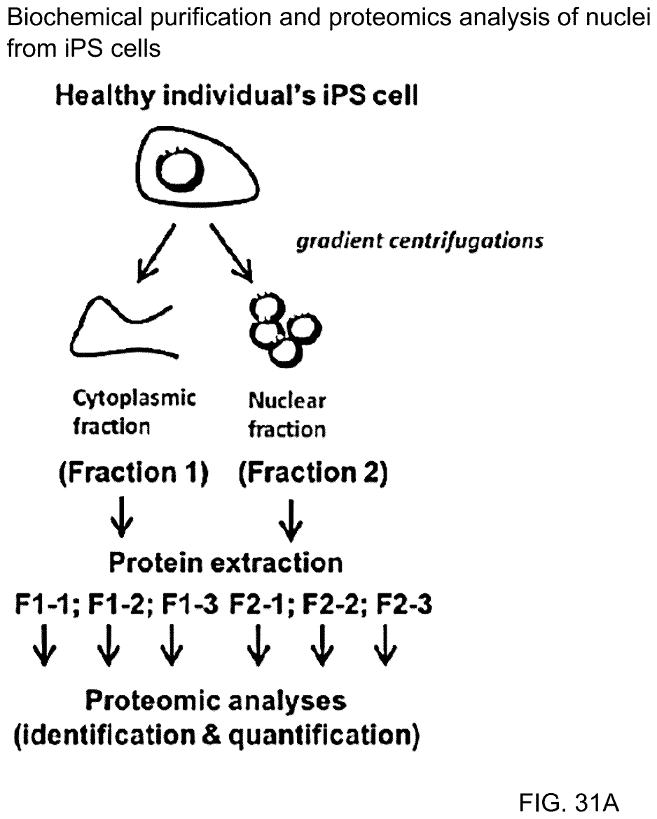

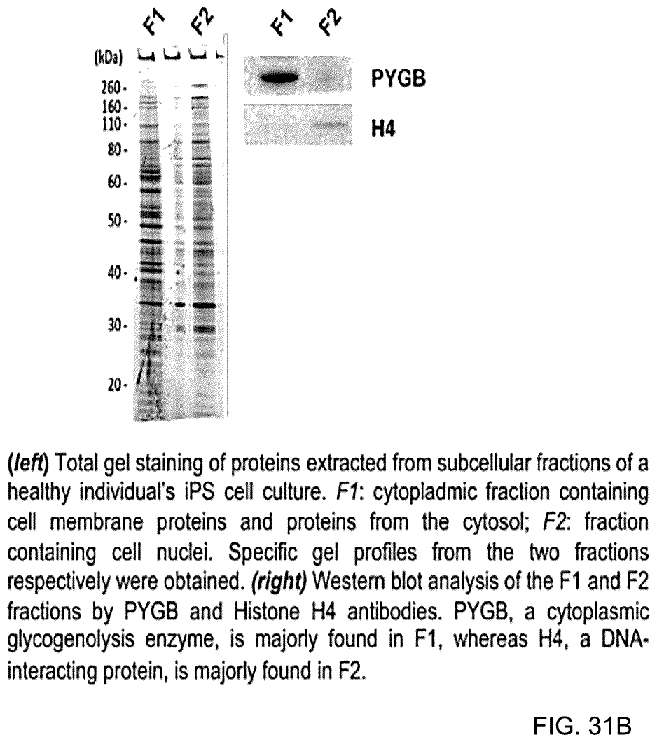

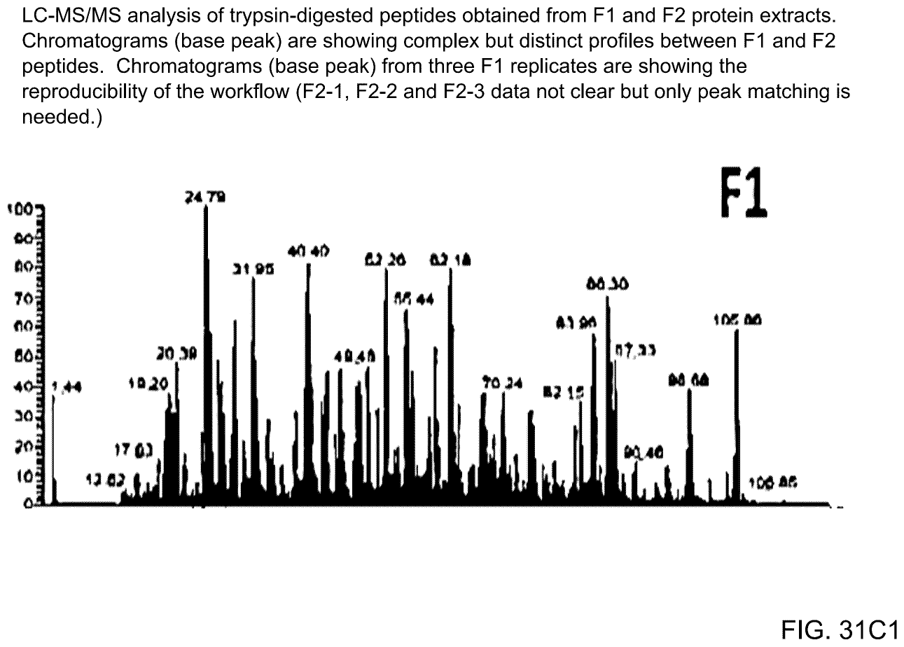

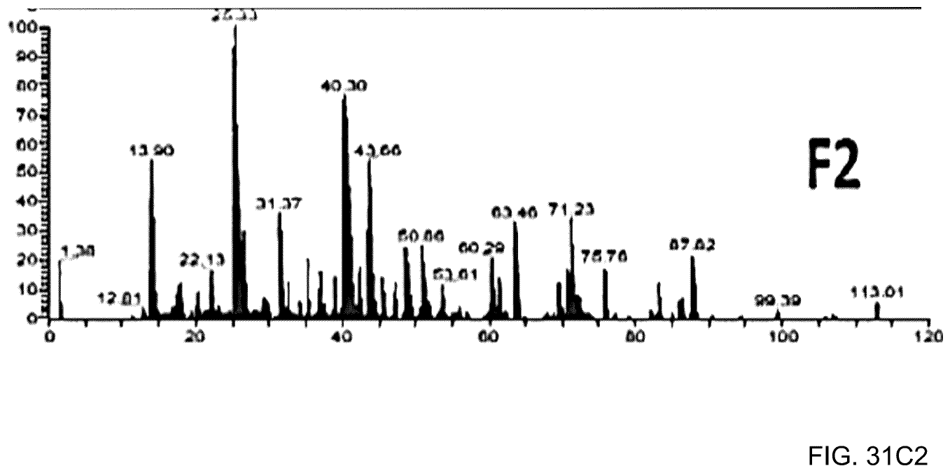

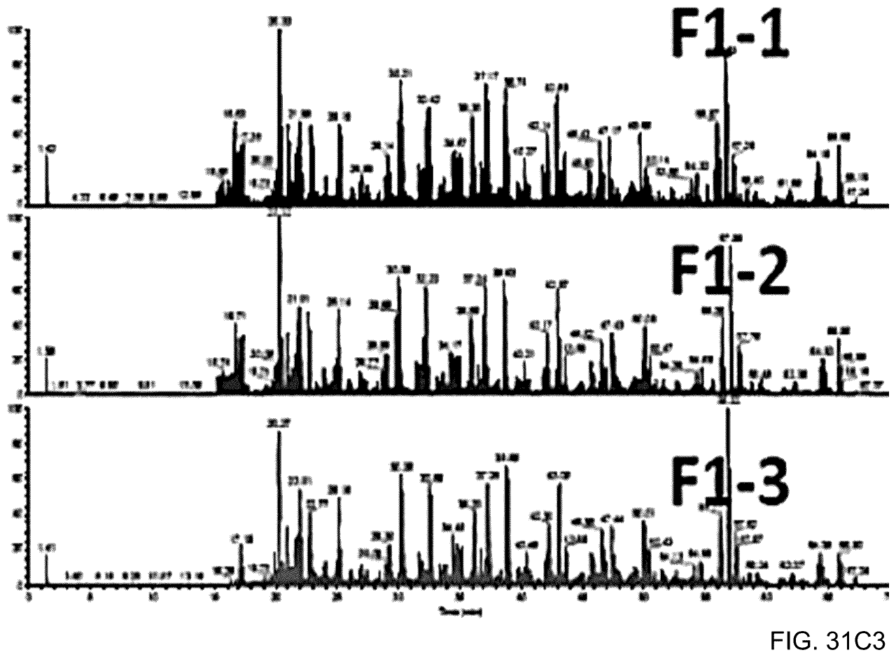

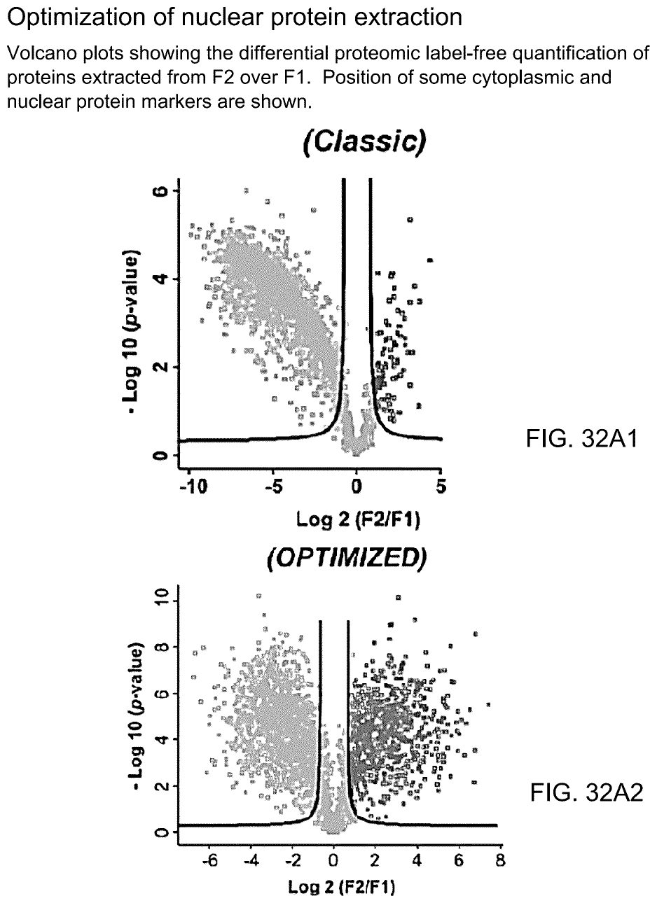

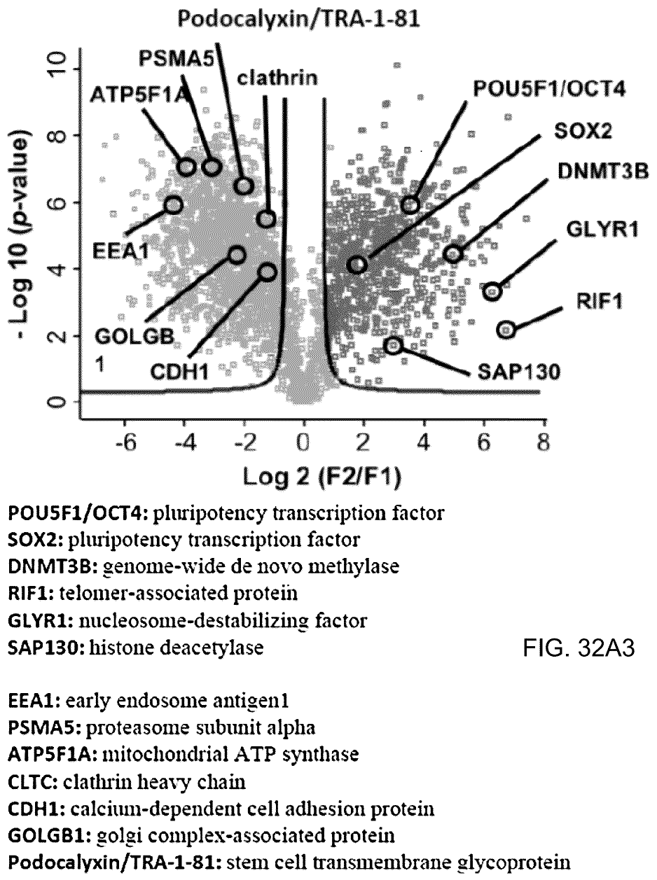

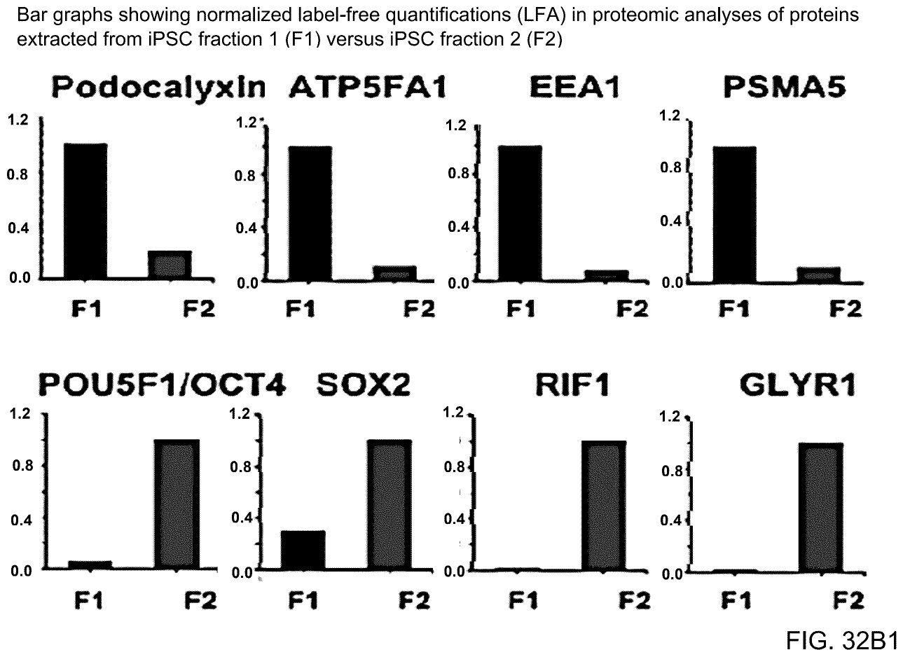

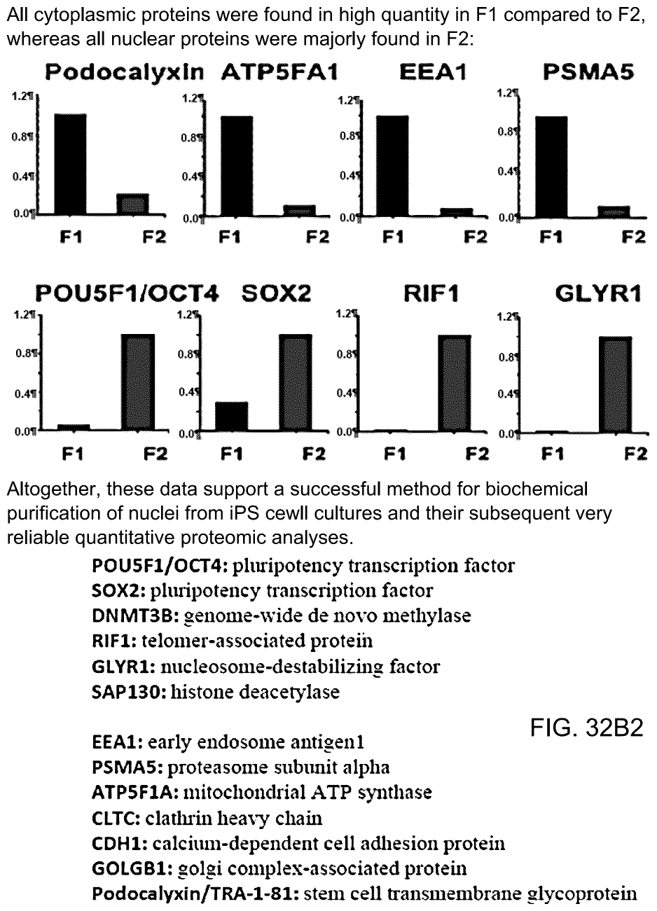

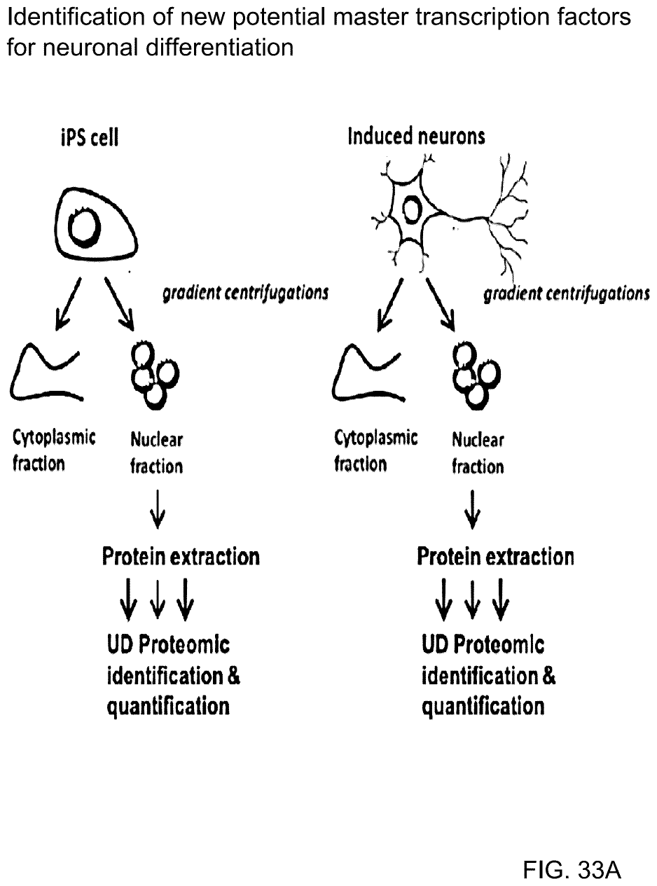

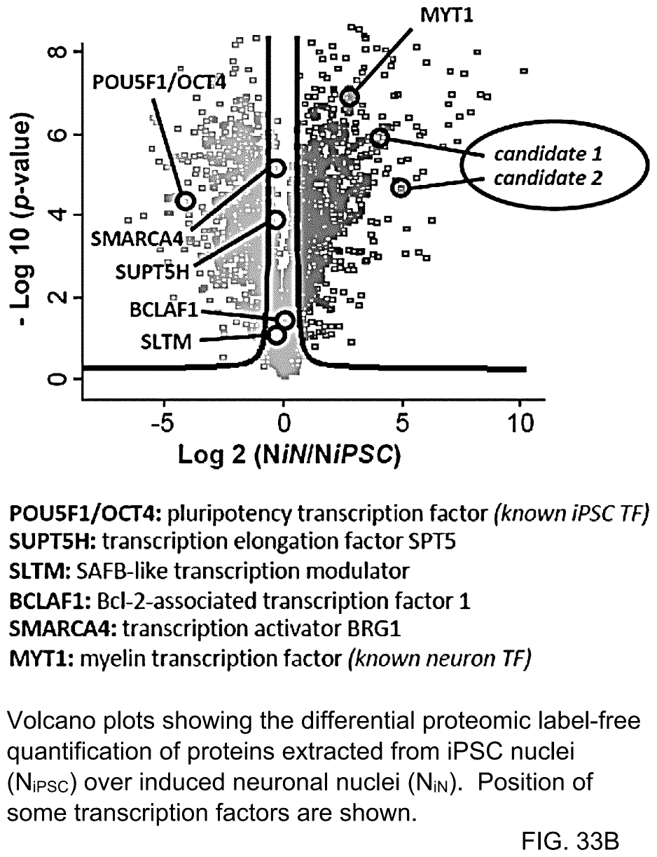

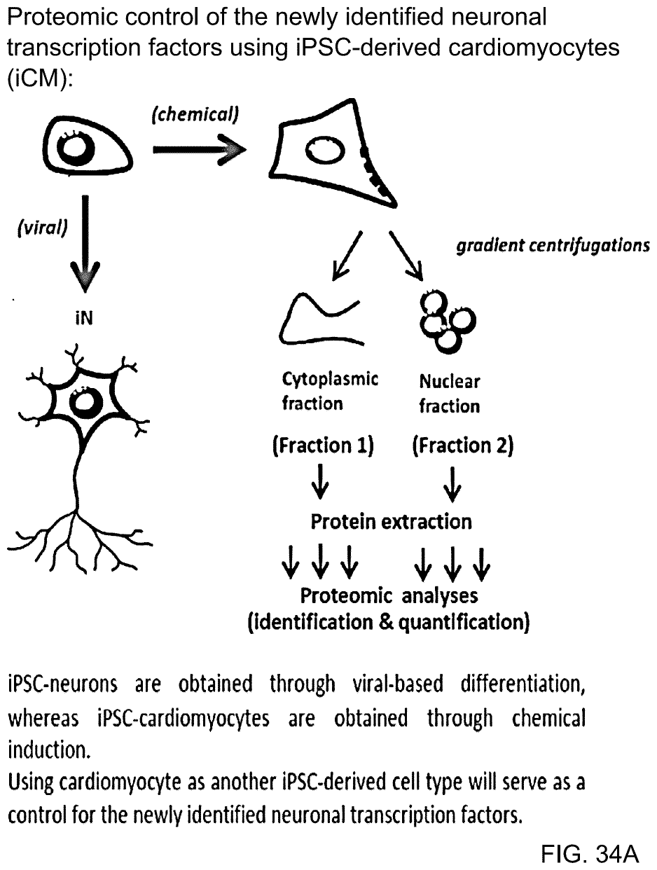

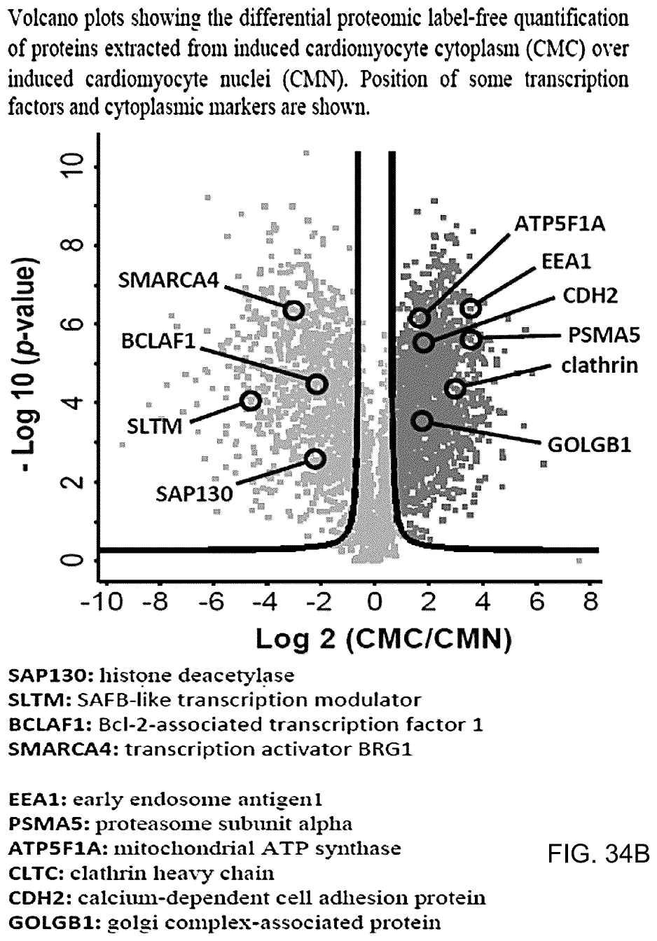

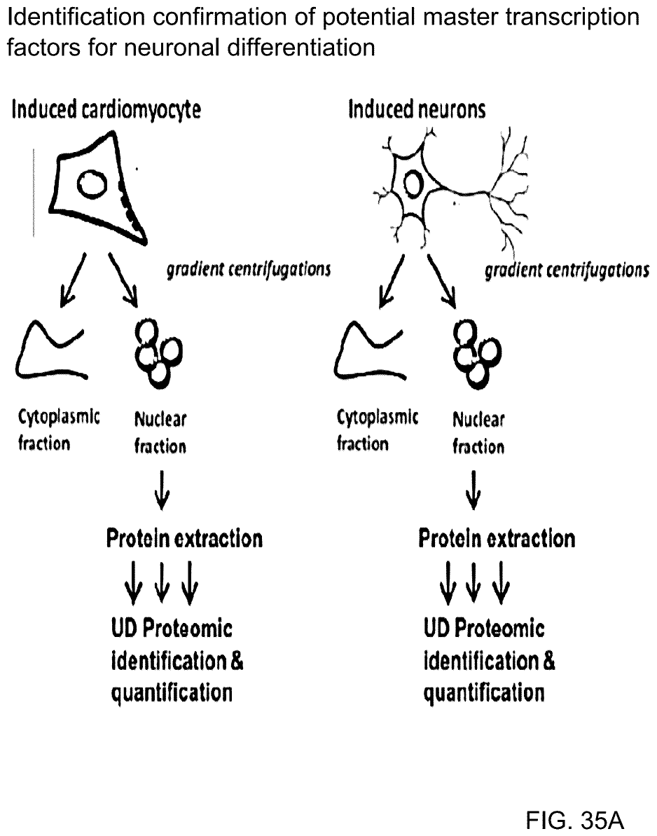

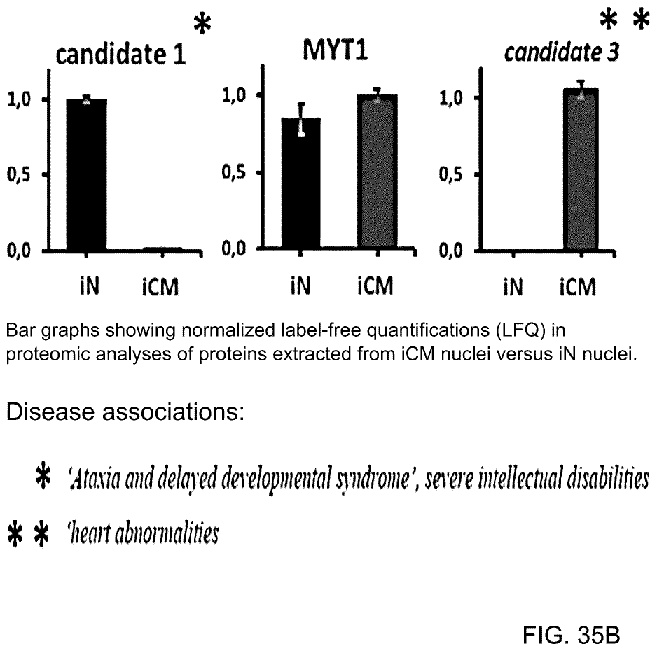

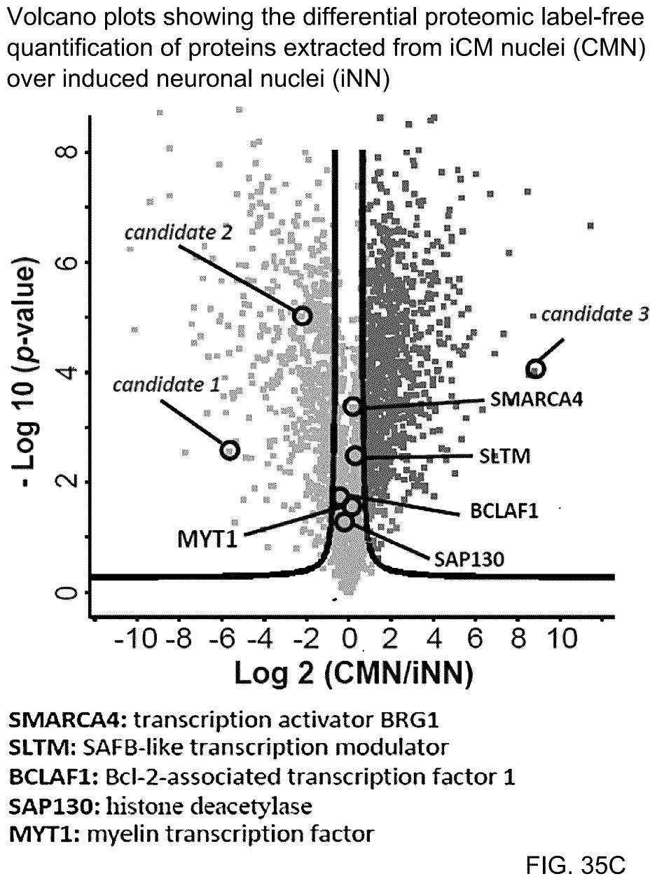

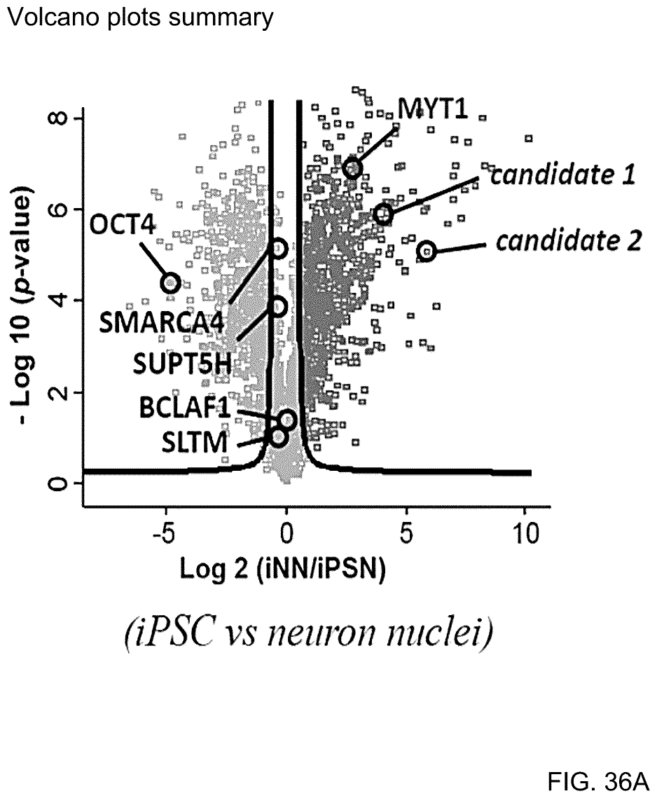

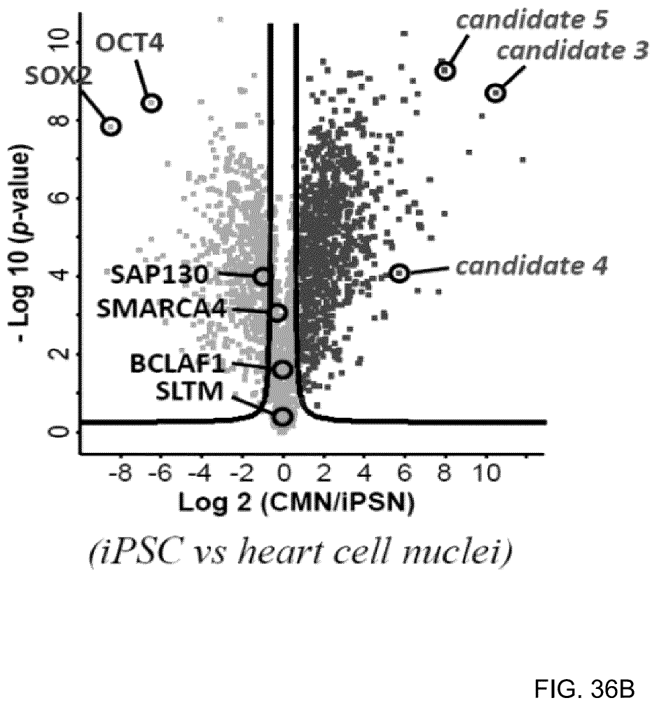

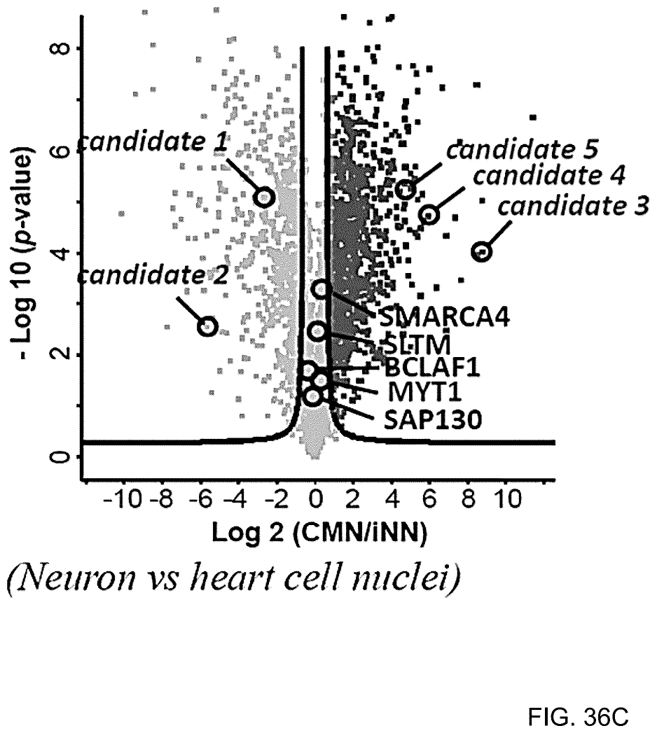

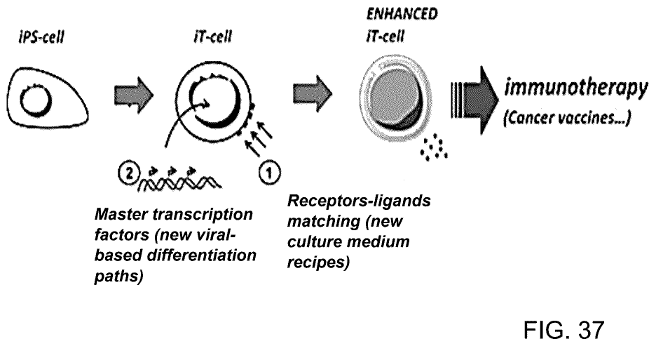

The fourth example is a newly discovered protein in the SV fraction (FIG. 4). In databanks, this protein is known as RGD1305455 (SEQ ID NO: 18; Uniprot ID: A0A0G2KAX2), or as ‘uncharacterized protein C7orf43 homolog’ and ‘similar-to-hypothetical protein FLJ10925’. Nothing is known regarding its tissue expression, developmental profile, or subcellular localization. Six unique peptides from RGD1305455 were detected only in UD experiments (FIG. 4A). RGD1305455 was found as an SV-resident protein (SV/P2′ ratio=3) of low abundance (rank 407; copy number/SV~0.04) (FIGS. 4A, 4B). It harbors a conserved DUF domain (DUF4707) and lacks a predicted transmembrane domain. Database searches revealed that the protein is highly conserved among mammals (>97% amino acid identity, FIG. 4C, FIG. 9). To confirm its presence in the SV fraction, we employed a targeted proteomic strategy. The UD unique peptide VLVVEPVK (SEQ ID NO: 19; FIG. 4A) was chemically synthesized using a ‘heavy’ C-terminal lysine (¹³C₆ and ¹⁵N₂) and mixed with a digested SV protein sample. A parallel reaction monitoring (PRM) assay based on elution time, ionization and fragmentation of the heavy peptide detected a matched VLVVEPVK peptide (SEQ ID NO: 19) in the SV sample (FIG. 4D). Close comparison between observed and expected peptide fragments (Table S4) indicated that mass errors of native fragments fell within 0.02 Dalton (FIG. 4D) confirming with high precision that protein RGD1305455 indeed exists in the SV fraction. Likewise, in PRM assays using 15 other heavy peptides for newly identified SV-resident proteins (Table S5), the presence of all the tested proteins in the SV fraction was confirmed.

### Functional Characterization of an SV-Associated Kinase Protein, Aak1

The SV fraction contained numerous non-transmembrane proteins, some of which reside with SVs within the synaptic compartment. These proteins might play a regulatory role in neurotransmission. To address this, we focused our analyses on protein kinases, which are mostly soluble cytoplasmic proteins. We identified AP2-associated protein kinase 1 (Aak1) as an abundant and SV-resident kinase. The copy number of Aak1 was calculated as 1.5/SV (See FIG. 7), suggesting ubiquitous presence among SVs in central synapses. The enriched profile of Aak1 in purified SV fraction was confirmed by western blot, contrasting with other cytoplasmic kinases found in P2′, such as MARK2 or TNiK. In cultured hippocampal neurons, strong co-localization of exogenously expressed Aak1 (TagRFP-Aak1) with a SV marker, synaptophysin was observed.

We employed both genetic and pharmacological approaches to clarify the functional role of Aak1, using shRNA knockdown (KD) of Aak1 expression in cultured hippocampal neurons, and by infusing an Aak1-specific inhibitor LP-935509 (32) directly into calyx of Held presynaptic terminals in brainstem slices of rats at postnatal day (P) 13-15. For Aak1-KD, we applied a lentivirus targeting Aak1 at day 11 in vitro (DIV11), when synaptophysin became detectable in western blot. At DIV15, the knockdown effect became maximal, reducing Aak1 expression below 5%. In hippocampal culture at DIV15, EPSCs in Aak1-KD neurons underwent a rapid short-term depression (STD) during stimulation at 20 Hz. The magnitude of STD was significantly greater than that in controls (p<0.05, n=7). Consistently, at the calyx of Held loaded with LP-935509, EPSCs underwent stronger STD during a 100 Hz train compared to controls (0.3 s, p<0.05, n=7). Cumulative histograms of EPSC amplitudes provided the pool size of readily releasable SVs and release probability, indicating that both Aak1-KD and Aak1 inhibitor reduced the pool size without affecting the release probability. Furthermore, the recovery from STD was prolonged, both at the Ca²⁺-dependent fast component (33) and Ca²⁺-independent slow component at the calyx of Held. These results together suggest that Aak1 normally facilitates SV recycling, thereby maintaining the releasable SV pool.

To further investigate whether Aak1 is involved in exo-endocytosis of SVs, we performed pHluorin assays in cultured hippocampal neurons and capacitance measurements at the calyceal terminal. In pHluorin assays, endocytic fluorescence half-decay time was prolonged by 2-fold (p<0.005, n=51) compared to controls (n=20). Likewise, in capacitance measurements, LP-935509 (1 or 10 UM) significantly prolonged the endocytic capacitance change. Capacitance measurements did not indicate a significant reduction of exocytosis. Thus, both at hippocampal and brainstem synapses, Aak1 likely plays an accelerating role in SV endocytosis.

Since the above results suggest involvements of Aak1 in the SV recycling pathway, we further investigated whether Aak1 might have a physiological role in the maintenance of neurotransmission. Simultaneous recordings of presynaptic and postsynaptic APs indicated that the Aak1 inhibitor (1 μM) significantly impaired the fidelity of neurotransmission, assayed as a ratio of postsynaptic APs generated in response to presynaptic APs (p<0.01, n=6). Altogether, our data indicate that Aak1 is a canonical SV-resident protein with an essential functional role in maintenance of neurotransmission, particularly at high frequency.

### Many Low Abundance SV Proteins are Linked to a Diverse Range of Physiological Functions Neurological Disorders

Many proteins were newly detected by UD proteomics in both high- and low-abundance ranges of the SV fraction proteome, with >80% found in lower ranges. Even though expressed at low abundance, SV proteins may play important physiological roles. We investigated this possibility using functional and disease annotations in our database, by classifying SV proteins into 17 functional categories with 26 subcategories (FIG. 5A). Our dataset contains trafficking proteins including SNAREs involved in various membrane fusions (#26) (2, 6). It also contains many types of Rab GTPases (#40) and membrane-tethering Trapp complexes (#14). Many of these proteins are identified as SV-resident, suggesting that SVs may be equipped with proteins for various trafficking routes toward other presynaptic organelles. Other major categories included proteins involved in signaling (e.g. kinases, phosphatases), signal transduction, and transport of small molecules. UD proteomics detected a high number of metabolic enzymes (#179) including those involved in neurotransmitter metabolism (#13), cellular energy production (#35), lipid regulation (#75), and cyclic nucleotide second messengers (#12). These data suggest the occurrence of metabolic reactions on SVs in crowded presynaptic terminals (6). UD proteomics also detected SV proteins categorized as autophagy-related proteins (#40). The presence of both SV-resident (e.g. snap29, atg9a, trappc8, pik3c3) and SV-visitor autophagy-related proteins (e.g. beclin-1, uvrag, map1lc3a, cisd2) in our SV proteome suggests that autophagic degradation may participate to the maintenance of SV population size within presynaptic terminals.

To examine pathological implications of our UD proteomics data, we searched for genetic information on SV proteins regarding their associations with neurological diseases (see ‘Diseases in the SV proteome’) and marked them in ranked abundance plots of SV (FIG. 5B) and P2′ fraction proteomes. We found that 236 different brain diseases are associated with 210 high- and low-abundance proteins of the SV proteome, of which 159 (76%) are newly revealed by the UD method. Likewise, 55% of these SV proteins was found in low abundance ranges of the P2′ proteome. These results indicate pathological significance of SV proteins irrespective of their abundance. SV protein-associated diseases include many motor (#145), cognitive (#135), and sensory system phenotypes, such as visual (#33) and auditory (#14) phenotypes. The database also indicates SV proteins associated with phenocopy diseases, such as mental retardation (#28), epilepsy (#25), Parkinson's disease (#13), amyotrophic lateral sclerosis (#4), Alzheimer's disease (#4), and cerebellar ataxia (#10). Our UD cross-analyses between functions and diseases indicate that phenocopies may involve proteins from both SV-resident and SV-visitor repertoires, from both high- and low-abundance ranges, and from functionally distinct proteins in the SV life cycle. For example, Parkinson's disease can be linked to mutations in SV-resident proteins such as renin-receptor (121st rank), involved in SV acidification, dnajc13 (318th rank, SV endocytosis) and sv2c (97th rank, SV trafficking), or in SV-visitor proteins, such as synaptojanin-1 (351st rank, SV endocytosis) or pla2g6 (653rd rank, lipid composition). Altogether, our data analyses illustrate the complexity and physiological importance of low-abundance SV protein repertoires newly detected by UD proteomics.

New Generation Proteomics with Improved Peptide Preparation Techniques have Revealed 3 Times Larger SV Proteome

To achieve a comprehensive SV proteome, maximal visibility of protein-specific sequences or ‘unique peptides’ prior to mass spectrometry (MS) identification seemed essential. Hence, we elaborated sequential enzymatic protein digestions by introducing LysC treatments before and during trypsin digestion to increase the number of accessible cleavage sites (FIGS. 2A and 6A). To maximally separate cleaved peptides, we utilized electrostatic repulsion-hydrophilic interaction chromatography (ERLIC) in addition to conventional reverse-phase chromatography (RPC). RPC separation of peptides is based upon hydrophobicity profiles, whereas ERLIC enables separations based on multiple biophysical properties of amino acids including their charges, polarities, isoelectric pH, post-translational modifications and structural orientations (Alpert, 2008; Alpert et al., 2010).

Due to technological advancement in the last decade, the resolution and sensitivity of mass spectrometers have increased greatly. To evaluate how much the machine development contributes to expand SV proteomics, we utilized a traditional peptide preparation protocol prior to MS analysis with a modern mass spectrometer (Q-Extrative Plus, FIG. 6A). We called this the ‘high-definition’ (HD) method (Gallien et al., 2012). In distinction, we named our new protocol of sample preparation and MS analysis as an ‘ultra-definition’ (UD) method (FIGS. 2B and 6). For MS analysis, the UD method enabled detecting of a larger number of unique peptide (e.g. 116 unique peptides for active zone protein Piccolo) than traditional methods (e.g. one unique peptide in (Takamori et al., 2006) and 14 unique peptides with the HD method, FIG. 2B). Remarkably, the UD method has revealed ~1500 proteins in the SV fraction purified from adult rat whole brain, tripling the number previously reported for SV proteome (Takamori et al., 2006). It also doubled the number of proteins revealed by the HD method (FIG. 2C).

The UD method increased not only the size of SV proteome, but also the number of isoforms identified within protein families, such as synaptotagmin family (FIG. 2D), in which only 5 isoforms were previously identified (Takamori et al., 2006). The HD method identified only one additional isoform, whereas the UD method revealed 9 additional isoforms, including synaptotagmin 6 of yet uncharacterized function (Chen and Jonas, 2017) and synaptotagmin 7, recently shown to be involved in multiple modes of neurotransmitter release (Liu et al., 2014; Jackman et al., 2016; Luo and Sudhof, 2017; Turecek et al., 2017).

In parallel, we also applied HD and UD methods for analyses of purified synaptosomal fraction (P2′) from adult rat whole brain and found that UD method markedly increases the number of proteins and their unique peptide coverages (FIG. 6A). For the quality control prior to our mass spectrometry experiments, we submitted our SV and P2′ samples to electron microscopy (EM) analysis, where we found uniform small SV structures in SV samples and well preserved subsynaptic structures in P2′ samples (FIG. 2E). We also examined protein profiles of SV and P2′ samples in SDS-PAGE (FIG. 2F) and western blot analyses.

Improved quantitative profiling of proteins in the subsynaptic compartments In quantitative mass spectrometry, protein abundance can be determined from unique peptide intensity using intensity-based absolute quantification (iBAQ), a label-free approach in which summed intensities of all unique peptides of a protein is divided by the total number of unique peptides detected. In this method, large number of unique peptides obtained by the UD method compared with the HD method should, in theory, increase the reliability of the protein quantification.

To test whether this is the case, we performed western blot analyses for 43 proteins in both the P2′ and SV fractions and compared the profiles between the HD and UD methods. As expected, proteins known to reside at the postsynaptic cell membrane or in the synaptic cleft were found in the P2′ but not in the SV fraction, both in western and MS analyses. Proteins known to reside on SVs were found at higher levels in the SV fraction than the P2′ fraction, in both western and UD analyses. In contrast, the HD method failed to detect some SV proteins, such as synaptogyrin 3, CSPa, SV31 and vMAT2, in the P2′ fraction. Similar inconsistencies between western profiles and HD iBAQ data were found for active zone (AZ) proteins, presynaptic membrane proteins, or cytoplasmic proteins. Via the UD method, the proteins in AZ, presynaptic membrane, cytosol, or in mitochondria were consistently detected with western profiles of subcellular fractions as reported previously (Takamori et al., 2006; Ahmed et al., 2013; Boyken et al., 2013). Altogether, mismatches between western blot profiles and iBAQ data were 30% (13/43) using the HD method, but none with the UD method, indicating that UD proteomics is the most reliable quantitative method at present for the synaptic proteomes.

UD quantification revealed spatial features and diversity of the SV proteome In subcellular fractionation experiments, western blotting is conventionally utilized for individual synaptic protein profiling. To gain a large-scale comprehensive distribution profile of SV-related proteins in the presynaptic compartment, we conducted UD quantitative cross analyses on equal amount of protein extracted from the P2′ and SV fractions. We identified 4,424 proteins in the P2′ and 1,466 proteins in SV fractions. A large group of 3,005 proteins were detected only in P2′, whereas a small group of 47 SV proteins were detected only in SV fraction (FIG. 3A). The former proteins can be defined as synaptosomal proteins having no direct interaction with SVs, which included post-synaptic and mitochondrial proteins such as PSD-95, gephyrin and cox-4. The latter 47 proteins, which included vGluT3, are likely proteins concentrated in a subset of SVs revealed by their purification, but presumably in too low abundance or peptide resolution to be detected in P2′ purified from total brain. 1,419 proteins were detected in both the P2′ and SV fractions.

We further characterized their distribution profile by quantifying their abundance from an SV/P2′ ratio using a volcano plot (FIG. 3B). We found that 134 proteins had SV/P2′ abundance ratios significantly higher than 2. We defined this group as the ‘SV-resident protein repertoire’ as it comprised all previously established proteins residing in SVs, such as synaptophysin, synaptotagmin-1, vacuolar-ATPase-related proteins, SV2, Scamp1, CSP-a, synapsin-1 and mover (Jahn and Sudhof, 1994; Morciano et al., 2005; Burre et al., 2006; Takamori et al., 2006; Ahmed et al., 2013). On the other hand, a majority of the 1419 proteins had a SV/P2′ abundance ratio lower than 1, suggesting that these reside mostly in other synaptic compartments, but also occasionally interact with SVs. We defined them as the ‘SV-interacting protein’ repertoire, as it comprised (i) cytosolic proteins, such as calmodulin, actin, and tubulin, (ii) active zone proteins, such as piccolo and bassoon, and (iii) plasma membrane proteins such as synaptojanin-1 and syntaxin 1, all of which are known to interact transiently with SVs (Hirokawa et al., 1989; McPherson et al., 1996; Igarashi and Watanabe, 2007; Jahn and Fasshauer, 2012; Boyken et al., 2013). Thus, UD quantitative proteomics can distinguish SVresident and SV-interacting synaptic proteins. These data provide spatial and functional bases for the numerous newly identified proteins in SV fraction by UD approach.

Proteins detected in either one of these repertoires may not co-exist on SVs in the same quantities. To address this issue, we used UD quantitative data to further analyze the amount and diversity of the 1,466 proteins detected in the SV fraction in a ranked abundance plot (FIG. 3C). As previously reported (Takamori et al., 2006), the transmembrane proteins synaptophysin, synaptotagmin 1, synaptobrevin-2, SV2A, vGluT1, the vATPase complex proteins and the lipid-anchored proteins synapsin-1 and rab3A, were highly abundant (word cloud chart in FIG. 3C) with 180 such proteins accounting for 90% of the total SV protein mass (ranked abundance plot in FIG. 3C). These SV proteins have iBAQ values >1.2×108 (1.2E8), whereas iBAQ of other 1,286 proteins ranged from E5 to E8. Copy numbers per SV are previously estimated for the highly abundant SV proteins, to build up an ‘average SV model’ (Takamori et al., 2006). We extended this estimate to less abundant SV proteins as well as to abundant but previously unresolved SV proteins, using isotope-labeled peptides. We utilized previously determined copy number of synaptotagmin 1 (15, (Takamori et al., 2006)) as a standard. Using this new method, we re-estimated the copy number of rab3A to be 10.5 (10 in (Takamori et al., 2006)), and newly estimated those of Aak1 (1.5), mover (0.94), Atg9A (0.04) and git1 (0.006) (Table S1). These results indicate that the copy numbers of many SV proteins are below 1, suggesting that they are present only in subpopulations of SVs (FIG. 3C). Although ~10% of SV proteins occupies 90% of SV protein mass (‘canonical repertoire’), less abundant numerous SV anchoring proteins seem to contribute to functional diversity of SVs.

In conventional EM imaging, purified SVs appear as uniform membrane structures of 40-50 nm in diameter (FIG. 2E, 3D). In conventional EM preparations, contrast enhancing reagents such as uranyl acetate are utilized. These reagents mainly bind membrane phospholipids, thereby making structures around SVs invisible. To visualize structures anchoring SVs, we used high-angle, annular, dark field scanning electron microscopy (HAADF-STEM, (Krivanek et al., 2010)) that has near-atomic imaging resolution without staining samples. In HAADF-STEM, purified 40 nm SVs were surrounded by massive and heterogeneous structures polarized on one side of the SVs (FIG. 3E, schematized in FIG. 3F). High magnification pictures indicated protein-like atomic structures. Adapter protein complex-2 (AP-2) is a presynaptic membrane protein involved in clathrin coating of SVs during endocytosis, and it may anchor various proteins to SVs. UD proteomics newly identified AP-2 as an abundant SV-resident adapter protein. Hence, based on our UD proteomics findings, we propose a new model for SV architecture that comprises SV-anchored proteins in addition to previously described core proteins (Takamori et al., 2006) (FIG. 3F).

### Power of UD Proteomics Method for Detecting Hidden SV Proteins

To explore hidden aspects of the SV proteome, we tabulated all proteins detected in the SV fraction by UD proteomics with annotations (see Supplementary excel database). Our new UD-SV proteomics data are compared with those reported by Takamori et al (2006), and can be filtered by gene families, abundance rank, and by structural information, such as transmembrane, lipid-anchored or soluble proteins and with cell functional categories, such as metabolic enzyme, small GTPases-related proteins, or autophagy-related proteins.

In the dataset, we first examined the rab subfamily of small GTPases, using the functional keyword ‘rabs’. Rab proteins play central roles in docking and targeting of transporting vesicles to specific subcellular organelles and membranes (Stenmark, 2009). Rabs are evolutionally conserved displaying 75-95% amino acid sequence identity (Diekmann et al., 2011). Such a high homology may complicate their proteomics detection. Using UD proteomics, we detected 40 rabs in the SV fraction, of which 32 are already documented, but without abundance quantification (Takamori et al., 2006). We found most rabs (29/40) in the SV-resident protein repertoire and in high abundance ranges, indicating that SVs may have intensive and variable trafficking routes within the presynaptic compartment other than endo-exocytosis. Rab11A and rab11B are some of the most identical isoforms with 91% of amino acid sequence identity (FIG. 4A). However, they are involved in opposite endosomal-sorting routes (Grimsey et al., 2016). We found 14 peptides common to both rab11A and B. However, only UD proteomics could detect a unique peptide of rab11A in the C-terminal hypervariable region (FIG. 4A). Thus, the UD method with improved peptide coverage can reveal highly homologous but functionally distinct proteins.

We next searched for subunits of vacuolar (v) ATPase protein complex (gene family: atp6, functional keyword in the dataset: ‘vATPase’). VATPases function as ATP-driven proton pumps for vesicle acidification, which is required for neurotransmitter uptake by synaptic vesicles. The vATPase complex is composed of two domains; a peripheral domain ‘V1’ composed of eight different proteins (A, B, C, D, E, F, G, H), and a membraneembedded domain ‘V0’ that is assembled with four different proteins (a, c, d, e) (Forgac, 2007) (FIG. 4B). Although previous proteomics studies estimated the copy number of vATPase as more than one per SV, they failed to identify the complete set of vATPase proteins in the SV fraction (Morciano et al., 2005; Burre et al., 2006; Takamori et al., 2006; Boyken et al., 2013). Here using UD proteomics, we identified all components of vATPase complex that were detected mostly in the high abundance range of the SV proteome (FIG. 4B, Supplementary excel database). Additionally, in lower abundance ranges, we identified previously detected vATPase accessory proteins such as Wdr7 and renin receptor (atp6ap2) as well as newly detected proteins such as Dmxl1 and Dmxl2 (Merkulova et al., 2015) (FIG. 4B). These low abundant accessory proteins likely regulate vATPase complex functions through transient interactions and/or in restricted subpopulation of SVs. Thus, the UD method may reveal complete inventories of protein complexes.

We next performed UD analyses of SV-resident transporter proteins. Solute carrier (‘slc’) transporters are transmembrane proteins that control the movement of soluble molecules across cellular membranes. At present, more than 400 slc genes are identified in mammals, of which ~40% are still uncharacterized with respect to their expression profile and/or function(s) (Cesar-Razquin et al., 2015). In our UD analysis, transporters detected in SV fraction were either SV-resident (i.e. iBAQ score in SV>P2′) or plasma membrane synaptic proteins (i.e. iBAQ score in SV<P2′), with the latter most likely being ‘caught into the traffic’ of vesicles during cycles of exo- and endocytosis (FIG. 4C). The SV-resident transporters include vGluT1 (slc17a7) and vGluT2 (slc17a6) for glutamate neurotransmitter uptake, and vGAT (slc32a1) for GABA and glycine uptake, which define molecular identities of the major SV populations in the brain (Takamori et al., 2000a; Takamori et al., 2000b), therefore they occur in high abundance in the SV proteome (FIG. 4C). UD proteomics also detected at lower abundance range, established SV-resident transporters that were missing in previous SV proteomics studies. These include vMAT2 (slc18a2, (Nirenberg et al., 1995)), ChT1 (slc5a7, (Ferguson et al., 2003)), vAchT (slc18a3, (Weihe et al., 1996)), and SVOP (atypical slc subfamily, (Janz et al., 1998)), involved in uptake of monoamine neurotransmitters (dopamine, serotonin, norepinephrine, histamine) or acetylcholine into relatively minor SV populations.

In addition to these well-known transporters, UD analyses further identified 9 new SV-resident transporters (FIG. 4C), of which Slc10a4 reportedly uptake bile acid into SVs to modulate dopamine activity (Larhammar et al., 2015). The remaining 8 transporters are entirely new and unknown in function (Table S2). Taken together, our data demonstrate that UD proteomics have unveiled hidden proteins of both high and low abundance in the SV proteome, present in canonical and subpopulations of SVs within the brain.

Identification and Confirmation of a Novel Mammalian SV Protein Discovered with UD Proteomics

We discovered an entirely new protein in SV fraction from adult rat brain. This protein of unknown function is named RGD1305455 (Uniprot ID: A0A0G2KAX2), ‘uncharacterized protein C7orf43 homolog’ or ‘similar-to-hypothetical protein FLJ10925’ in databanks. Six unique peptides generated from RGD1305455 were detected in LC-MS/MS analyses in the UD method, whereas none were detected in any of the SV and P2′ HD experiments. Our UD analyses indicated that the product of this uncharacterized gene is an SVresident protein, likely present in a subset of vesicles or synapses within the brain. At present, there is no information available from gene databanks regarding the gene expression in tissue, its developmental profile, or subcellular localization of the protein. RGD1305455 is a non-transmembrane protein having a conserved DUF domain (DUF4707) in eukaryotes. By comparing homologs reported in other vertebrates, we found a high conservation of the amino acid sequence (>97% identity) among mammals (FIG. 9), suggesting that the structure and function of RGD1305455 were evolutionarily conserved among animals with highly integrative brain functions.

In complex biological mixtures, a total of 500,000 peptides on average may be detected and sequenced by mass spectrometry. Identification artefact resulting from overlapping peptide spectra and false positive protein assignment may occur in LC-MS/MS analyses (Chen et al., 2009; Bogdanow et al., 2016). To exclude the possibility that RDG1305455 detected in UD proteomics is an artefact, we employed a targeted proteomics strategy using synthetic stable isotope-labeled peptide to confirm the presence of native peptides composing this protein in digested SV protein samples.

The unique peptide sequence VLVVEPVK (SEQ ID NO: 19) detected in UD proteomics was chemically synthesized incorporating a fully labeled (13C6 and 15N2) at the C-terminal lysine (K), resulting in a mass shift of +8 Da (‘heavy peptide’). This heavy peptide was then analyzed by LC-MS/MS prior to its mixing with a digested SV protein sample, to obtained information on its elution time, ionization and fragmentation patterns. A parallel reaction monitoring (PRM) assay (Peterson et al., 2012) based on these parameters detected a matched native VLVVEPVK peptide (SEQ ID NO: 19) in the SV sample. Close comparison between observed and expected peptide fragments (Table S4) indicated that mass errors of the native fragments fell within 0.02 Dalton confirming the existence of RGD1305455 protein in the SV fraction with high precision. We also performed PRM assays using 15 other heavy peptides for newly identified SV-resident proteins (Table S5). The presence of all the tested proteins in the SV fraction was confirmed. These results provide strong evidence for a hitherto ‘hidden SV proteome’ with potentially important functions.

### Functional Identification of an SV-Associated Kinase Protein, Aak1

The SV fraction contained numerous transmembrane or lipid-anchored proteins as well as ‘soluble accessory’ proteins that may stay attached to SVs within the synaptic compartment (see Supplementary excel database). These SV accessory proteins may play significant regulatory role in neurotransmission. To address this possibility, we focused our UD data analyses on protein kinases, which are mostly soluble cytoplasmic proteins. Using the keywords: ‘signaling’ and ‘kinase’, we identified Aak1 from our database as the kinase most strongly attached to purified SVs (FIG. 5A). The copy number of Aak1 was calculated as 1.5/SV (FIG. 7), suggesting its ubiquitous presence among SVs in central synapses (FIG. 5B). The highly enriched profile of Aak1 in purified SV relative to P2′ fractions was confirmed by western blot, contrasting with profiles of other cytoplasmic kinases found in synapses (P2′) such as MARK2 and TNiK. In cultured hippocampal neurons, using confocal fluorescence microscopy, we observed a strong co-localization of both exogenously expressed Aak1 (TagRFP-Aak1) and major SV component synaptophysin (sypHy) in neuronal terminals (FIG. 5C). These data support the notion that Aak1, although it is a soluble kinase protein, localizes to synaptic vesicles within the presynaptic compartment.

We employed both molecular/genetic and pharmacological approaches to clarify the functional role of Aak1, using shRNA knockdown (KD) of Aak1 expression in cultured hippocampal neurons, and an Aak1-specific inhibitor LP-935509 (Kostich et al., 2016) directly loaded into the calyx of Held presynaptic terminals in brainstem slices of rats at postnatal day (P) 13-15. For shRNA knockdown of Aak1, we applied lentivirus targeting Aak1's exon 11 (Ultanir et al., 2012) at Day 11 in vitro (DIV11), when synaptophysin became detectable in western blot. We used a non-efficient shRNA targeting Aak1's exon 20 as a negative control (FIG. 5D). At DIV15, the knockdown effect became maximal, reducing Aak1 expression below 5% in western blot analysis. We recorded EPSCs from hippocampal neurons at DIV15 and induced a short-term depression (STD) using a train of 20 stimulations at 20 Hz. Compared with controls, EPSCs in Aak1-KD neurons showed a more rapid and stronger STD, noticeable within a couple of stimulations, and their amplitude declined to a significantly lower level (p<0.05, n=7, FIG. 5E). Likewise, at the calyces of Held loaded with LP-935509, EPSCs underwent a stronger STD during a 100-Hz train (0.3 s, p<0.05, n=7). At the calyx of Held, recovery after STD follows bi-exponential time course, with a Ca²⁺-dependent fast component and a Ca²⁺-independent slow component (Wang and Kaczmarek, 1998). The Aak1 inhibitor significantly prolonged time constants of both components (FIG. 5F). These results together suggest that Aak1 normally plays a facilitatory role for recycling and reuse of SVs in presynaptic terminals.

To further investigate whether Aak1 is involved in exo-endocytosis of SVs, we expressed the pH-sensitive fluorescent marker pHluorin coupled with synaptophysin (sypHy) in cultured hippocampal neurons and measured intra-vesicular pH changes associated with SV exocytosis and endocytosis (Miesenbock et al., 1998). In cultured hippocampal neurons with Aak1-KD, the magnitude of exocytosis, assayed via SV pH neutralization, was unchanged, but endocytic re-acidification time was prolonged by 2-fold in half-decay time compared to control cells (p<0.01, n=51 in Aak1-KD, n=20 in control) (FIG. 5G). At the calyx of Held, exo-endocytosis can be directly monitored using membrane capacitance measurements (Sun and Wu, 2001; Yamashita et al., 2005). In the presence of LP-935509 (1 or 10 UM) in presynaptic terminals the endocytic capacitance change was significantly prolonged (p<0.05, n=6), whereas presynaptic Ca²⁺ current charge (Q_{Ca}) or exocytic capacitance change (ΔCₘ) remained unchanged (FIG. 5H). Thus, consistent results at hippocampal and brainstem synapses suggest that Aak1 plays an accelerating role in SV endocytosis and recycling.

The above results indicate that reduced expression or inhibition of Aak1 has no direct effect on exocytosis, but that it impairs the recycling reuse of SVs. We then addressed whether Aak1 might play a physiological role in the maintenance of high-frequency neurotransmission. To this end, we recorded APs simultaneously from a presynaptic terminal (input) and a postsynaptic neuron (output) and evaluated the fidelity of neurotransmission (output/input AP ratio) at the calyx of Held (FIG. 5I). During 100-Hz stimulation, presynaptic APs did not fail at all (data not shown), but postsynaptic APs gradually failed. In the presence of LP-935509 (1 μM) in presynaptic terminals, postsynaptic APs started to fail earlier than those in controls and transmission fidelity declined to a significant lower level (~50%) within 40 s (p<0.01, n=6). Altogether, our data indicate that Aak1 is a canonical SV-resident protein within synapses, and that it plays an essential functional role for maintenance of high-frequency neurotransmission.

Mostly hidden (deep) proteomes may underlie brain diseases phenocopies Many proteins were newly detected by UD proteomics in both high and lower abundance ranges of the SV proteome, with most of them (~80%) being found in lower ranges (beyond 410th rank, iBAQ<2.7E7, see Supplementary excel database). Low abundance proteins that may not be ubiquitously present among SVs in central synapses may potentially play important physiological roles. To address this possibility, we examined functional annotations and diseases associations reported in our dataset. The ~1500 SV fraction proteins can be involved in a wide variety of cellular functions.

We classified the proteins into 17 categories with 26 subcategories. Our dataset contains a high number of trafficking proteins including diverse sets of SNAREs involved in various membrane fusions (26), as previously reported (Takamori et al., 2006; Burre and Volknandt, 2007; Wilhelm et al., 2014). It also contains a multitude of rab GTPases (40) and membrane tethering complexes trappc (14). Most of these proteins were identified in our UD proteomics as SVresident, suggesting that SVs are equipped for various trafficking routes and communications with other presynaptic organelles. Other major categories included proteins involved in signaling (e.g. kinases, phosphatases), signal transduction (e.g. trimeric GTPases) and in transport of small molecules. UD proteomics detected a high number of metabolic enzymes (179) including those involved in neurotransmitters metabolism (13), cellular energy production (35), (phospho) lipids regulation (75), as well as cyclic nucleotides (12).

These data suggest that the metabolic reactions in crowded presynaptic terminals (Wilhelm et al., 2014) likely happen locally in direct interactions with SVs. UD proteomics also detected SV proteins categorized as autophagy-related proteins (40). Some of these proteins are reportedly associated with neuropsychiatric diseases such as schizophrenia or Alzheimer's disease (Salminen et al., 2013; Vijayan and Verstehen, 2017). The presence of both SV-resident (e.g. snap29, atg9a, trappc8, pik3c3) and transiently interacting SV proteins (e.g. beclin-1, uvrag, map1lc3a, cisd2) from all ranges in our SV proteome (see Supplementary excel database), indicates that control of SV population size by autophagy, may have an important physiological role at synapses.

To reveal connections to pathologies in our UD proteomics data, we searched for proteins detected in the SV fraction having reported neurological disease(s) caused by mutation(s) in those genes (see Supplementary excel database for ‘Diseases in the SV proteome’) and marked them in the ranked abundance plot. We found 236 brain diseases associated with 210 proteins detected in both high and low abundance ranges of the SV proteome. Of 210 SV proteins, 159 (76%) proteins were newly revealed by the UD method. SV protein-associated diseases comprised many motor (145) and/or cognitive (135) diseases, and a relatively smaller number of sensory diseases, including visual (33) and auditory (14) dysfunctions.

The database also includes SV proteins linked to a high number of phenocopy diseases, such as mental retardation (28), epilepsy (25), Parkinson's syndromes (13), amyotrophic lateral sclerosis (4), Alzheimer's disease variants (4), and ataxia (10). Our UD cross-analyses between functions and diseases indicate that phenocopies may be caused by proteins from both SV-resident and SV transiently-interacting repertoires, from high and low abundance ranges and from proteins involved in distinct functions of the SV life cycle. For example, Parkinson's syndromes may be linked to mutations in SV-resident proteins such as renin-receptor (121st rank, SV acidification, (Korvatska et al., 2013)), dnajc13 (318th rank, SV endocytosis, (Vilarino-Guell et al., 2014)) and sv2c (97th rank, SV trafficking, (Hill-Burns et al., 2013)), or in SV transiently-interacting proteins, such as synaptojanin-1 (351st rank, SV endocytosis, (Quadri et al., 2013)) and pla2g6 (653rd rank, SV lipid composition, (PaisanRuiz et al., 2009)) (see Supplementary excel database for ‘Diseases in the SV proteome’). Altogether, our data analyses illustrate the complexity and functional importance of protein repertoires newly detected by UD proteomics in the low abundance range of the SV proteome.

### EXAMPLES

Hereinafter, the present invention will be described more specifically based on the following examples. It should be noted that this embodiment does not limit the present invention.

### Materials and Methods

All animal experiments were performed in accordance with guidelines of the Physiological Society of Japan, the German Animal Welfare Act, and regulations at the Okinawa Institute of Science and Technology, at the Max-Planck Institute for Biophysical Chemistry, and at Doshisha University.

### Brain Synaptosomes and Synaptic Vesicles Purifications:

Synaptosomes (P2′) and synaptic vesicles (SV) were purified from whole brain of 4-6-week-old Sprague Dawley rats following the same protocols used in (2) and previously described in (34) for SV, and in (4) for P2′. The quality of all P2′ and SV purification procedures was controlled by western blots of synaptic protein markers and by electron microscopy. Extended descriptions of biochemical, imaging and electrophysiological procedures and analyses are provided in SI Materials and Methods.

### UD Proteomics Sample Preparation and Mass Spectrometry:

### Sequential Protein Digestion:

Fifty micrograms of proteins extracted from P2′ or SV were resuspended into 200 μL of buffer containing 8 M urea, 100 mM Tris-HCl (pH=8) (‘urea buffer’) and placed onto a Pall Nanosep (Registered trademark) 10K Omega filter (Sigma). After shaking 1 min at 850 rpm at room temperature (Eppendorf Thermomixer), samples were centrifuged during 13 min at 6,400×g (conditions that were optimal to remove all the liquid from the upper chamber using a TOMY Kintaro KT-24 centrifuge). After repeating these steps two times, proteins were resuspended with 200 μL of urea buffer containing 50 mM iodoacetamide and incubated in darkness for 1 hr at room temperature. The alkylation was then stopped by centrifuging as above and by resuspending protein samples with 200 μL of urea buffer containing 25 mM DTT. Unfolded proteins were subsequently washed with 20 mM ammonium bicarbonate. Proteolytic enzymes were used in a ratio of 1:50 with proteins. To generate peptides, a first digestion step (‘trimming’) was performed using endoproteinase lys-C(Promega) for 6 hrs at 37° C., followed by a second digestion step overnight (16-18 hrs) at 37° C. using a trypsin/lys-C combination (Promega). After centrifugation as above, digested peptides were acidified with 1% TFA, concentrated and dried using an EZ-2 Elite evaporator (SP Scientific).

### Orthogonal Peptide Separations:

To separate peptides, off-line electrostatic repulsion-hydrophilic interaction chromatography or ERLIC-based separation was performed (Alpert et al., 2010). The following conditions were adapted and optimized to obtain the highest number of identified proteins from P2′ and SV samples. Mobile phase solvents preparation: solvents were freshly prepared for each experiment using LC/MS Grade acetonitrile (ACN), formic acid (FA) and water from Thermo Fisher Chemicals. Solvent A: 90% ACN, 0.1% FA. Ammonium hydroxide (NH₄OH, 25% w/w in water, Fluka) was then added to adjust pH at 4.5. Solvent B: 30% ACN, 0.1% FA. The digested peptide mixture was resuspended with 20 μl of solvent A, and injected into on a weak anion exchange PolyWax LP column (PolyLC Inc.; 1 mm inner diameter×150 mm, 5 mm particle size, 300 A pore size) using a PAL HTC autosampler (CTC Analytics) for automatic injection and fractions collection, using a gradient mode (3 min solvent A, to 10% B in 7 min, 10% B to 25% B in 24 min, 25% B to 70% B in 16 min, 70% B to 81% B in 6 min, 81% B to 100% B in 3 min, with final wash at 100% B for 6 min and re-equilibration at 100% A for 20 min) at a flow rate of 40 μL/min. Twenty four fractions were collected every 3 min between 0 and 72 min, and subsequently concentrated to dryness using speed vacuum Genevac EZ-2 Elite (SP Scientific).

### Mass Spectrometry:

Dried peptides were resuspended in 30 μl of 0.1% formic acid and analyzed using a Q-Exactive Plus Orbitrap hybrid mass spectrometer (Thermo Scientific) equipped with an Ultimate 3000 nano-high-pressure liquid chromatography (nano-HPLC) system (Dionex), HTC-PAL autosampler (CTC Analytics), and nanoelectrospray ion source. Five microliters of each sample were injected into a Zorbax 300SB C18 capillary column (0.3×150 mm, Agilent Technologies) and heated at 40° C. A one-hour HPLC gradient was employed (1% B to 32% B in 45 min, 32% B to 45% B in 15 min, with final wash at 75% B for 5 min and re-equilibration at 1% B for 10 min.) using 0.1% formic acid in distilled water as solvent A, and 0.1% formic acid in acetonitrile as solvent B. A flow rate of 3.5 L/min was used for peptide separation. Temperature of the heated capillary was 300° C., and 1.9 kV spray voltage was applied to all samples. The mass spectrometer settings were as follow: full MS scan range 350 to 1500 m/z with a mass resolution of 70,000, 30 μs scan time, and automatic gain control set to 1.0E6 ions, and fragmentation MS2 of the 20 most intense ions.

### Protein Identification:

Protein identification was done using Proteome Discoverer software v2.1 (Thermo Scientific), and Mascot 2.6 (Matrix Science) as a search engine. A database downloaded from UniprotKB Rattus norvegicus (Proteome ID: UP000002494) was used with search parameters as follow: trypsin enzyme, up to two miscleavages, with precursor and fragment mass tolerance set to 10 ppm and 0.02 Da respectively. Cysteine carbamidomethylation, methionine oxidation, asparagine and glutamine deamidation, and N-terminal protein acetylation were set as variable modifications. The results were filtered using a false discovery rate of <1% as a cutoff threshold, determined by the Percolator algorithm in Proteome Discoverer software.

### Quantitative Proteomic Data Statistical Analyses:

For the volcano plot, intensity-based absolute quantification (iBAQ) data obtained from Proteome Discoverer were used for statistical analysis using R software version 3.2.5 (R Project for Statistical Computing). Quasi-Poisson generalized linear models were generated (y~1, y~ treat) and compared using analysis of deviance for generalized linear model fits (Anova) to obtain p-values, using an F-test, and adjusted with Benhamin-Hochberg method.

### Data Availability.

Proteomic raw data files data have been deposited in the Japan Proteome Standard Repository Database (accession no. JPST000968). All other study data are included in the article, Dataset S1, and SI Appendix.

### Supplementary Information Text

### SI Materials and Methods

Purification of Synapses and Synaptic Vesicles from Rat Brain:

Synaptosomes (P2′) and synaptic vesicles (SV) were purified from 4-6 weeks old rat brains. All steps were performed at 4° C. Nine brains were dissected out and homogenized in 60 mL of ice-cold sucrose buffer (0.32 M sucrose, 4 mM HEPES NaOH, pH 7.4, supplemented with 1 μg/mL pepstatin and 0.2 mM PMSF protease inhibitors) in a glass-teflon homogenizer using 9 up-and-down strokes at 900 rpm. The brain homogenate (BH) was centrifuged 10 min at 2,700 rpm 4° C. (Sorvall SS34 rotor). The resulting pellet (P1: cell debris, nuclei) was discarded, while the supernatant (S1) was collected and centrifuged for 15 min at 10,000 rpm 4° C. (Sorvall SS34 rotor) to obtain the cytosolic fraction (S2) and the crude synaptosomal fraction (P2). A Ficoll gradient was prepared in sucrose buffer with the following layers (from bottom to top): 4 mL of 13% Ficoll, 1 mL of 9% Ficoll, and 4 mL of 6% Ficoll. The P2 fraction (3 mL in each tube) was layered over the Ficoll gradient and centrifuged for 35 min at 22,500 rpm 4° C. (SW41 rotor, Beckman). The fraction at the interface between the 13% and the 9% Ficoll layers was collected, diluted in sucrose buffer and centrifuged for 12 min at 11,000 rpm 4° C. (Sorvall SS34 rotor).

The pellet was then resuspended in sucrose buffer to obtain the synaptosomes fraction (P2′). For SV purification, P2 suspension was additionally centrifuged for 15 min at 10,500 rpm 4° C. (Sorvall SS34 rotor), and pellet was resuspended in 13 mL of sucrose buffer. This suspension, referred to as ‘well-washed’ crude synaptosomal fraction, was then transferred to a glass-teflon homogenizer. Osmotic lysis was immediately performed by adding 117 mL of ice-cold water and 3 up-and-down strokes at 3,000 rpm. The resulting synaptosomal lysate was buffered with 1 mL of 1 M HEPES-NaOH (pH 7.4) and centrifuged 20 min at 16,500 rpm 4° C. (Sorvall SS34 rotor) to yield a lysate pellet (LP1, synaptic membranes-enriched fraction) and a lysate supernatant (LS1, cytoplasmic content of synapses). LS1 was then collected, transferred to 12 10-mL polycarbonate tubes and centrifuged for 2 hrs at 50,000 rpm 4° C. (50Ti rotor, Beckman).

The supernatants (LS2) were removed, and the pellets (LP2, ‘crude synaptic vesicles’ fraction) were resuspended in 3 mL of 40 mM sucrose. The suspension was then layered on top of a continuous 2%-22% (w/v) sucrose gradient in 5 mM HEPES pH 8.0 (generated using an automatic gradient mixer, Biocomp Instruments), and centrifuged for 4 hrs at 25,000 rpm 4° C. (SW28 rotor, Beckman). The fractions corresponding to synaptic vesicles-enriched sucrose regions (=material banding at 7%-13% sucrose) were collected, pooled, and layered on top of a controlled-pore glass chromatography column (2 cm internal diameter×150 cm) (glass beads: mean pore diameter of 300 nm, 74-125 μm (120/200 mesh) in size). The size exclusion chromatography was performed overnight in glycine buffer (0.3 M glycine, 5 mM HEPES-NaOH pH 7.2, 0.02% sodium azide) at a flow rate of 40 mL/hr. Fractions containing predominantly heterogeneous membranes with diameters exceeding 100 nm were excluded. Fractions containing uniformly shaped small vesicles with 40-45 nm diameter were then collected and centrifuged for 1 hr at 50,000 rpm 4° C. (SW50.1 rotor, Beckman) to obtain the ‘pure synaptic vesicles’ fraction (SV). The quality of all P2′ and SV purification procedures was controlled by western blots of synaptic protein markers and by electron microscopy (see ‘Western blotting characterization’ and ‘Electron microscopy imaging’).

### Protein Extraction and Immunoblotting Characterization:

Proteins were extracted using a lysis buffer containing 100 mM Tris-HCl (pH=8), 4% SDS, 100 mM DTT, and protease inhibitor cocktail (Sigma). Protein concentrations were analyzed by using both NanoDrop (Trademark) 2000 (Thermo Scientific) and Direct Detect (Registered trademark) (Millipore) spectrophotometers. Proteins from each fraction were loaded equally (10 μg in each lane) onto 4-12% Bis-Tris SDS gel (NuPAGE(Trademark), Thermo Scientific), and transferred to a supported nitrocellulose membrane (Bio-Rad). The transferred membrane was blocked with 5% Skim Milk TBST for 1 hr at room temperature. All primary antibodies (see Antibodies Table) were used in 1% Skim Milk TBST at dilution 1:1000 and incubated with membranes overnight at 4° C. After three washes in TBST, secondary antibody-HRP conjugate was used at dilution 1:2000. After washes, blots were developed using Clarity (Trademark) Western ECL Substrate (Bio-Rad Laboratories) and imaged on ChemiDoc (Trademark) XRS+ system with Image Lab (Trademark) Software (Bio-Rad Laboratories).

### Targeted Proteomics Analyses:

### Peptide Selection Criterions

We selected peptide sequences for targeted proteomics according to the following criterions: 1) unique peptide from synaptic protein detected by HD and/or UD proteomics; 2) peptide which did not include any amino acid modification such as acetylation or carbamidomethylation; 3) peptide with less than 16-amino-acid-length which allowed rapid synthesis at high purity without additional liquid chromatography purification steps.

### Peptide Synthesis

The peptides were synthesized through conventional 9-fluorenylmethyloxycarbonyl (Fmoc) solid-phase peptide synthesis (SPPS), onto preloaded Fmoc-¹³C₆¹⁵N₂lysine or Fmoc-¹³C₆ ¹⁵N₄ arginine TCP-resins (Intavis Bioanalytical Instruments), on a 1 μmole scale in 96-well plate and using a high-throughput automated peptide synthesizer ResPep SL (Intavis Bioanalytical Instruments). All Fmoc-amino acids were purchased from Watanabe Chemical Industries and prepared at 0.5 M in N-methyl pyrrolidone (NMP, Wako Pure Chemical Industries). For the automated synthesis, the following reagents (Wako Pure Chemical Industries) were dissolved in N, N-dimethylformamide (DMF) and used according to Intavis Bioanalytical Instruments SPPS protocol: 0.5 M HBTU coupling reagent, 44% N-methyl-morpholine (NMM) base, 5% acetic anhydride capping reagent, and 20% piperidine deprotecting reagent. After synthesis, peptides were cleaved with (v/v/v) 92.5% TFA, 5% TIPS and 2.5% water for 2 hrs, precipitated using t-butyl-methyl-ether at −30° C., pelleted and resuspended in water before lyophilization (EYELA FDS-1000) overnight. Before use, weighted peptides were re-dissolved in water and their concentration was confirmed using a Direct Detect (Registered trademark) infrared spectrophotometer (Millipore), the calibration curve of which was generated with peptide standards (6×5 LC-MS/MS Peptide Reference Mix, Promega). All synthesized peptide purity and sequence were then confirmed by LC-MS/MS (see ‘Mass spectrometry’) and information on their elution time, m/z value, major charge state, and fragmentation information were collected using parallel reaction monitoring (PRM).

De Novo Sequencing and Correlation with Isotope-Labeled Reference

To confirm the presence of novel SV proteins in samples, peptide de novo sequencing analysis results from MS/MS data and correlation with the synthetic peptides (spectrum annotations, ion match tables, plotted mass errors) were performed using PEAKS software (v 7.0, Bioinformatics Inc).

### Targeted Proteomics Analyses

The PRM acquisition method combined two scan events corresponding to one full scan and one PRM event targeting the doubly and triply charged precursor ions of the synthesized peptides (154 reaction masses). The full scan event employed a mass range from 350-1200 m/z, an orbitrap resolution of 70,000, a target automatic gain control (AGC) value of 5E5, and a maximum injection time of 30 ms in profile mode. The full scan event was followed by a PRM with 3 multiplexed scan events, which employed an orbitrap resolution of 17,500, a target AGC value of 1E6 and a maximum injection time of 50 ms.

The precursor ion of each targeted peptide was isolated using a 1.6-m/z unit window and a positive offset of 0.4-m/z. Fragmentation was performed with stepped collision energy of 15, 22, 27 and MS/MS scans were acquired with a starting mass of 300 m/z, the ending mass being automatically defined by the charge state of the precursor ion. The generated MS/MS scan libraries were uploaded into Skyline software (version 3.6.0.10493) (1) and all the assigned fragment ions were extracted. For each SRM chromatogram, the automatic peak integrations and the native over heavy abundance ratio within peak boundaries were calculated using the software.

### Absolute Quantification

Absolute quantification was performed by mixing known quantities of synthetic heavy peptides (0.1 or 0.05 μg) with 50 μg of digested SV proteins. The absolute quantities of the native peptides were calculated using the abundance signal from their corresponding heavy peptides. The absolute quantity (μg)=(0.1 or 0.05)×(native/heavy abundance ratio). Average copy number of a protein per SV was estimated using synaptotagmin 1 (Syt1), a transmembrane SV-resident protein, as a reference (15 Syt1 per SV, estimated by Takamori et al): Protein #/SV=absolute quantity/Molecular Weight×[(Syt1 #/SV)×(Syt1 Molecular Weight)/(Syt1 absolute quantity)].

### Protein Annotations in the SV Proteome Dataset:

For each protein identified in the SV fraction, a search in the scientific literature (PubMed, Google Scholar) and the databases (UniprotKB, NCBI) was performed manually. The most significant biological function(s) were reported, and functional keyword(s) were assigned for data filtering purpose. The structural annotations related to protein-membrane interactions were made using UniprotKB. Proteins were referred to as having transmembrane domain(s) (TM) or not, and for membrane-anchored proteins, the type of lipidation was indicated. A comparison between our data and the previously described SV proteome by Takamori et al was performed.

As multiple names designating a given protein are frequently encountered in protein taxonomy and in the scientific literature (e.g. the tumor protein p63-regulated gene 1-like protein (Tprg1l) is also known as being Mossy fiber terminal-associated vertebrate-specific presynaptic protein (Mover) and Family with sequence similarity 79 member A protein (Fam79A)), we proceeded to rigorous cross-comparisons through amino acid sequence retrieval from the listed gi numbers and NCBI RefSeq by Takamori et al and the Uniprot ID-associated amino acid sequence and gene symbol from our SV protein data set. Proteins were then referred to as having been previously detected in Takamori et al or not. The disease annotations were made using the Uniprot and GeneCards databases for human diseases.

Proteins detected in the SV fraction having ‘disease(s) caused by mutation(s) affecting the gene represented in the entry’ were indicated. The descriptions of syndromes were used to categorize the reported diseases into cognitive, motor and/or sensory neurological disorders. Additionally, we searched in the recently published Synaptic Gene Ontologies resource SynGO (2) for potential contaminations by postsynaptic-specific proteins in the SV fraction. Proteins were then referred to as being reported in SynGO or not and SynGO cellular components were indicated.

### ‘Word Cloud’ Representation:

A script was used in Python to generate a list of iBAQ values and Uniprot names of the 400 most abundant proteins in SV-1 experiment, each normalized by the iBAQ value of synaptophysin (rank 1st in SV-1). The list was uploaded into the web interface of an online word cloud generator WordArt (formerly TagUI) to generate the SV proteome word cloud image.

### Amino Acid Sequences Alignment:

The amino acid sequences of proteins were retrieved from Ensembl and aligned using the ClustalW2 program at EMBL-EBI. Alignment output and shading of the amino acids were processed using the Boxshade program (ExPASy Bioinformatics Resource Portal).

### Electron Microscopy Imaging:

### Synaptosome (P2′) Samples

Purified synapses were resuspended and fixed for 30 min at room temperature in 2.5% glutaraldehyde, 0.1 M cacodylate buffer. After three times washes with 0.1 M cacodylate buffer and centrifugations for 5 min 13,000 rpm (Eppendorf Centrifuge 5418), synapses were stained with 1% osmium 0.1 M cacodylate buffer for 30 min and centrifuged 5 min 3,000 rpm (Eppendorf Centrifuge 5810 R). Synapses were then washed three times with 2 ml of pure water (Otsuka Distilled Water), and centrifuged 5 min 3,000 rpm, before successive dehydration steps in ethanol 70%, 80%, 90%, 95%, and three times in ethanol 100%. EPON resin solution was prepared on the day prior to use by mixing for 12 hrs TAAB 812, DDSA, MNA and DMP30 (TAAB Laboratories) in a ratio of 20:10:10:1, respectively. The resin solution was then mixed in a 1:1 ratio with 100% ethanol and used to resuspend and incubate synapses for 30 min at room temperature. After centrifugation as above, synapses were resuspended in the resin solution and mixed thoroughly for 10 min. A centrifugation for 1 min 1,000 rpm and incubations in a low vacuum chamber for 1 hr and overnight on bench were performed to remove micro-air-bubbles. Samples were then centrifuged for 30 min 5,000 rpm to collect a maximum of synapses on one edge of the resin, and put into oven for 2 days at 60° C. (Electron Microscope Oven TD-700, Dosaka EM co. Ltd.). Sections of samples (50 nm thickness) were performed using a Leica UC6 ultramicrotome. Synapse slices were mounted to copper grids (HF34 Maxtaform Grids 200 mesh, Nisshin EM co. Ltd.) pretreated with 100% acetone. Slices were then stained for 30 min with 4% uranium acetate, washed four times with pure water (Otsuka Distilled Water), and stained for 5 min with a lead solution (lead nitrate (II) 1%, lead acetate trihydrate 1%, lead citrate n-hydrate 1%, from Wako Pure Chemical Industries Ltd, pH 7 adjusted with NaOH) using Nalgene (Registered trademark) 171-0045 syringe filters and washed four times as above. Synapses images were collected on a JEM-1230R electron microscope (JEOL) operated at 100 keV and processed with Digital Micrograph software (Gatan).

### Synaptic Vesicle (SV) Samples

Carbon-copper grids were prepared as follow: a 5-15 nm carbon film (resistance of 4 ohm/cm, purity of 99.9999%, Nisshin EM co. Ltd.) was put on copper grids (HF34 Maxtaform Grids 200 mesh, Nisshin EM co. Ltd.) using a JEOL IB-29510VET device and pretreated by glow discharge in an ion coater (DII-29020HD, JEOL) to render hydrophilic and prevent particle agglomeration during drying. Purified synaptic vesicles (3 μl, of 20 times diluted from the original sample solution, in order to observe individual vesicles) were then deposited on grids, immediately dried with paper and stained with 1% uranium acetate (phospholipids contrast agent). SV images were collected on a JEM-1230R electron microscope (JEOL) operated at 100 keV and processed with Digital Micrograph software (Gatan).

### Fluorescence Microscopy Imaging:

### SypHy and Aak1 Cloning

SypHy-P2A-TagRFP and SypHy-P2A-TagRFP-Aak1 were expressed in a neuron specific manner by using lentivirus-based vectors, in combination with Tet-Off system (3). Two vectors were used, a ‘regulator’ vector expressing an advanced tetracycline transactivator (tTAad) under the control of human synapsin1 promoter (STB), and a “response” vector (TGB) that expressed SypHy-P2A-TagRFP or SypHy-P2A-TagRFP-Aak1 under the control of a modified tetracycline-response element (TRE) composite promoter. To construct SypHy-P2A-TagRFP-Aak1, a full-length mouse Aak1 (accession no. NM_001040106) was amplified by PCR and subcloned into a StuI site of pCR-Blunt vector (Thermo Fisher Scientific) according to manufacturer instruction and the sequence was verified. The full-length of Aak1 was excised by BamHI/EcoRI double digestion and cloned into a BgIII/EcoRI site of pTagRFP-C vector (Evrogen) in frame. Independently, a DNA fragment encoding sypHy lacking a stop codon (4) and a DNA fragment of a self-cleaving P2A peptide (5) were amplified by PCR and cloned into TGB vector by using In-Fusion cloning kit (Clontech) according to manufacturer instruction. Finally, a fragment encoding SypHy-P2A and that encoding TagRFP-Aak1 were PCR amplified and cloned into TGB vector by using In-Fusion cloning kit. To generate SypHy-P2A-TagRFP, essentially the same procedure was conducted except a TagRFP fragment amplified by PCR was conjugated with SypHy-P2A by using In-Fusion cloning kit.

### Lentiviral-Mediated Expression of SypHy and Aak1

Lentivirus were produced from HEK293T cells transfected with 3.4 μg of lentiviral backbone vector (either STB or TGB with SypHy-P2A-TagRFP/TagRFP-Aak1) and helper plasmids (pCAG-kGP1 2 μg, pCAG4-RTR2 1 μg and pCAG-VSVG 1 μg) (3) using a calcium phosphate transfection method (6). Cultures were infected with STB-lentivirus at 0-1 DIV and TGB-lentivirus at 7 DIV, and subjected to experiments at 14-16 DIV.

### Image Analysis

Live imaging was carried out at room temperature (~24° C.) on an inverted microscope (Olympus) equipped with a 60× (1.35 NA) oil immersion objective and 75 W Xenon lamp. Images (1024×1024 pixels) were acquired with a cMOS camera (ORCA-Flash 4.0, Hamamatsu Photonics) with 100 ms exposure time under the control of MetaMorph software (Molecular Devices). SypHy was imaged with 470/22 nm excitation and 514/30 nm emission filters, whereas TagRFP fluorescence was imaged with 556/20 nm excitation and 600/50 nm emission filters. Acquired images were analyzed using MetaMorph software. For quantifying TagRFP and SypHy fluorescence, line scan function in MetaMorph software was used. A Line with 5 pixels width was drawn along an axon manually, and fluorescence signals were normalized by the highest signals of either TagRFP or SypHy fluorescence in the selected region.

pHluorin-Based Live Imaging:

### Cloning

For pHluorin assay with knockdown of Aak1 expression, GFP in pLVTHM was replaced by the red fluorescent protein FusionRed (FusRed) through restriction-ligation cloning. FusRed was amplified from pCAG-FusRed template by PCR using KOD-plus-Neo DNA polymerase kit (Toyobo) adding the restriction sites MauB1 and Spe1 to the following primers: 5′-TCGACGCGCGCGGCCACCATGGTGAGCGAGCTG-3′ (forward; SEQ ID NO: 35), 5′-TATGACTAGTAT TTACCTCCATCACCAG-3′ (reverse; SEQ ID NO: 36). Amplified DNA fragment was run on agarose gel before purification with Monarch DNA Gel Extraction Kit (New England BioLabs), ligated to MauB1-Spe1-digested pLVTHM (T4 DNA ligase, New England BioLabs), and cloned in HST08 E. coli strain (Stellar (Trademark) competent cells, Clontech). Confirmed by sequencing plasmids were purified from E. coli using Plasmid Maxi kit (Qiagen). pLVTHM-FusRed-shRNA Lentiviruses were then prepared as described in ‘Lentivirus preparation and titration’.

### Phluorin Assay

Dissociated hippocampal neurons were transfected at DIVO with 0.8 μg of pCAG-SypHy2× plasmid by electroporation (one pulse at 1360V, 24 ms, for a 10 μL suspension of 100,000 cells) using Neon Transfection System (Invitrogen). After growing, cells were infected at DIV11-12 with pLVTHM-FusRed lentiviruses expressing the shRNA (see ‘shRNA cloning and knockdown assay’). Before imaging, cells cultured on a glass coverslip (DIV15-19) were placed on the built-in imaging chamber of the confocal microscope and continuously perfused with standard extracellular solution at 25° C. containing (in mM): 140 NaCl, 2.4 KCl, 10 HEPES, 10 glucose, 2 CaCl₂), 1 MgCl₂, 0.01 CNQX (pH 7.4). SypHy imaging was performed on a Zeiss LSM780 confocal microscope with a C-Apochromat 40×/1.2 W Korr M27 objective. Neurons were identified using SypHy resting fluorescence (neurons expressing synaptophluorin) and FusRed fluorescence (neurons expressing shRNA) with GFP band pass filter 488 nm excitation and 493-586 nm emission, and mCherry band pass filter 561 nm excitation and emission 578-697 nm, respectively. A concentric bipolar electrode (FHC), placed 80-100 μm away from the neuron, delivered a train of pulses (1 ms, 8V) at 10 Hz for 10 s. Image acquisition was carried out on a portion of axon of the stimulated neuron, in time-lapse mode, at 1-2 frames per second, through Zen software v2.1 (Zeiss). Baseline fluorescence was recorded for 1 minute before each stimulation.

### Image Analysis

Image analysis was performed using ImageJ (National Institutes of Health), and OriginPro2017 (OriginLab Corporation). In ImageJ, square regions of interest of 1.6 μm side were positioned manually at the centre of fluorescence puncta, and the corresponding fluorescence data was extracted to Origin. Fluorescence time course of raw traces were corrected for photo-bleaching with the fitted baseline fluorescence intensity F₀ as (F-F₀)/F₀ at each time point by OriginPro2017. Half-decay time was measured as the time required by the signal to reach half of the peak intensity after stimulation. The average half decay time data from 51 boutons of 16 neurons over 5 independent experiments for Aak1 knockdown was compared with average half decay tine data from 20 boutons of 10 neurons over 4 independent experiments.

### Electrophysiological Assays:

### Brain Slice Preparation and Solutions

Wistar rats (postnatal day 13-15) of either sex were killed by decapitation under isoflurane anesthesia. Transverse brainstem slices (175-200 μm in thickness) containing the medial nucleus of the trapezoid body (MNTB) were cut in ice-cold solution containing (in mM): 200 sucrose, 2.5 KCl, 26 NaHCO₃, 1.25 NaH₂PO₄, 6 MgCl₂, 10 glucose, 3 myo-inositol, 2 sodium pyruvate, and 0.5 sodium ascorbate (pH 7.4 when bubbled with 95% O₂ and 5% CO₂, 310-320 mOsm) by using vibroslicer (VT1200S, Leica). Before recording, slices were incubated for 1 h at 37° C. in standard aCSF solution containing (in mM): 125 NaCl, 2 KCl, 26 NaHCO₃, 1.25 NaH₂PO₄, 2 CaCl₂), 1 MgCl₂, 10 glucose, 3 myo-inositol, 2 sodium pyruvate, and 0.5 sodium ascorbate (pH 7.4 when bubbled with 95% O₂ and 5% CO₂, 310-320 mOsm), and maintained thereafter at room temperature (24-26° C.). MNTB principal neurons and calyx of Held presynaptic terminals were visually identified using a ×40 water immersion objective attached to an upright microscope (BX51WI, Olympus).

### Membrane Capacitance Measurement

Membrane capacitance measurement from the calyx of Held presynaptic terminals, in whole-cell configurations, were made at room temperature (RT, 26-27° C.). Data were acquired at a sampling rate of 50 KHz, using an EPC-10 patch-clamp amplifier controlled by PatchMaster software (HEKA) after on-line filtering at 5 KHz. Calyx of Held terminals were voltage-clamped at a holding potential of −80 mV and a sinusoidal voltage command with a peak-to-peak voltage of 60 mV was applied at 1 KHz. Aak1 inhibitor LP935509 (Axon MedChem) was dissolved in DMSO (0.1%), which was also included in pipette solution, and infused from whole-cell pipettes into calyceal terminals by diffusion. Care was taken to keep the access resistance below 14 M f ¶ to allow diffusion of the drug into the terminal within 5 min after whole-cell rupture. To isolate presynaptic voltage-gated calcium charge transfer (Q_{Ca}), the aCSF contained 10 mM tetraethylammonium chloride (TEA, Tokyo Chemical Industry Ltd.), 0.5 mM 4-aminopyridine (4-AP, Nacalai Tesque), 1 mM tetrodotoxin (TTX, Nacalai Tesque), 10 mM bicuculline methiodide (Santa Cruz Biotechnology) and 0.5 mM strychnine hydrochloride (Tokyo Chemical Industry Ltd.). Intracellular solution for presynaptic terminals contained (in mM): 125 Cs-methanesulfonate, 30 CsCl, 10 HEPES, 0.5 EGTA, 12 Na₂-phosphocreatine, 3 MgATP, 1 MgCl₂, 0.3 Na₂GTP (315-320 mOsm, pH 7.3 adjusted with CsOH). Tips of recording pipettes were coated with dental wax (GC Corporation) to reduce stray capacitance (4-6 pF).

Single-pulse step depolarization to +10 mV for 20 ms was used to induce presynaptic Q_{Ca}. Membrane capacitance (Cₘ) changes within 450 ms after square-pulse stimulation were excluded from analysis to avoid contamination with conductance-dependent capacitance artifacts. Data were obtained within 20 min after whole-cell rupture. The amplitude of exocytic Cm change (ΔCₘ) was measured as the difference of Cm values between the baseline and those at 450-500 ms after depolarization. Sample Cm records are shown as average values of each 50-data point (for 50 ms) plotted every 50 ms (for shorter time scale) or every 500 ms (for longer time scale). The half decay time of endocytosis was measured from the midpoint of ΔCₘ decay.

### EPSC Recording

For recording of evoked EPSCs, simultaneous pre- and postsynaptic whole-cell recordings were made from a calyceal nerve terminal and postsynaptic cell. Throughout the experiments, presynaptic recordings were made in current-clamp mode, whereas postsynaptic recordings were made in voltage-clamp mode at a holding potential of −70 mV. Pipette solution for recording of presynaptic action potentials (APs) contained (mM): 110 K-gluconate, 10 L-glutamate, 30 KCl, 10 HEPES, 0.5 EGTA, 12 Na₂-phosphocreatine, 3 MgATP, 1 MgCl₂, 0.3 Na₂GTP (315 mOsm, pH 7.3 adjusted with KOH), and that for postsynaptic recording contained (mM): 110 CsF, 30 CsCl, 10 HEPES, 5 EGTA, 1 MgCl₂, 5 QX314-CI (300 mOsm, pH 7.3 adjusted with CsOH). EPSCs were evoked by current injection (0.5-1 nA, 1 ms) into the presynaptic terminal via a recording glass electrode, in the presence of bicuculline methiodide (10 UM) and strychnine hydrochloride (0.5 μM).

### Action Potential Recordings

For recording postsynaptic action potentials (APs), simultaneous pre- and postsynaptic whole-cell recordings were made from calyceal terminals and postsynaptic MNTB principal neurons, both in current-clamp mode, in the presence of bicuculline methiodide (10 UM) and strychnine hydrochloride (0.5 μM). Pipette solution for postsynaptic AP recording contained (mM): 120 K-gluconate, 30 KCl, 5 EGTA, 12 Na₂-phosphocreatine, 3 MgATP, 1 L-arginine, 1 MgCl₂, 0.3 Na₂GTP (315 mOsm, pH 7.3 adjusted with KOH). Presynaptic APs were elicited by a square pulse current injection into calyces in current-clamp mode, via recording glass electrodes filled with K-gluconate-based internal solution (as above).

### Data Statistical Analysis

Data were analyzed using IGOR Pro 6 (WaveMetrics), Excel 2011 (Microsoft) and SigmaPlot 12 (Systat Software Inc.). All values are given as mean±S.E.M., and p<0.05 was taken as a significant difference in Student's t-test, one-way ANOVA with the Bonferroni post-hoc test.

### Hippocampal Cell Culture Electrophysiology

Whole-cell patch-clamp recordings were made from dissociated hippocampal cultures at DIV15 (earliest time for a complete Aak1 knockdown in neuronal cells infected by lentiviruses at DIV11-12, and culture developmental stage from which endogenous expression of major SV protein synaptophysin is detected). The pipette solution contained (in mM): 110 CsF, 30 CsCl, 10 HEPES, 5 EGTA, 1 MgCl₂, 5 QX314-CI (300 mOsm, pH 7.3 adjusted with CsOH). Cells were continuously perfused with standard aCSF solution containing (in mM): 125 NaCl, 2 KCl, 26 NaHCO₃, 1.25 NaH₂PO₄, 2 CaCl₂), 1 MgCl₂, 10 glucose, 3 myo-inositol, 2 sodium pyruvate, and 0.5 sodium ascorbate (pH 7.4 when bubbled with 95% O₂ and 5% CO₂, 310-320 mOsm) with 0.05 D-AP5, 0.01 bicuculline methiodide. The stimulating bipolar electrode was positioned close to the afferent neuron ~80-100 μm distant from the target neuron visualized with GFP under fluorescence microscope. Neurons were voltage clamped at −70 mV with an EPC-10 amplifier (HEKA Electronics, Germany). Only cells with series resistances of <15 milliohm, with 70-80% of this resistance compensated, were analyzed. Currents were acquired using PATCHMASTER software (HEKA Electronics), filtered at 5 kHz, and digitized at 10 kHz. Data were analyzed using AxographX (Axograph Inc., USA), and IgorPro (WaveMetrics Inc., USA). All experiments were carried out at room temperature. All values are given as mean±S.E.M., and p<0.05 was taken as a significant difference in Student's t-test, paired t-test, one-way ANOVA with the Bonferroni post-hoc.

### Dissociated Hippocampal Cell Culture:

Primary hippocampal cell cultures were performed following the description from (7). Neonatal pups (P1) of mice (ICR CD-1, Charles River Laboratories) were sacrificed by decapitation and hippocampi were dissected out at 4° C. in filter-sterilized HBSS buffer containing 0.1% glucose (Thermo Scientific), 1 mM sodium pyruvate (Thermo Scientific), and 10 mM HEPES (Sigma). Hippocampal tissue was then digested using the papain-based Neuron Dissociation Solutions S kit (Wako Pure Chemical Industries Ltd). Harvested cells were plated on poly-L-lysine coated μ-Dish 35 mm low (Ibidi) with Basal Medium Eagle (Thermo Scientific) containing 0.45% glucose, 1 mM sodium pyruvate, 2 mM L-glutamine (Thermo Scientific), and supplemented with 10% (v/v) fetal bovine serum (Thermo Scientific) and placed at 37° C. under 5% CO₂. After 3 hrs, the plating medium was replaced by maintenance medium: Neurobasal (Trademark) Medium (Thermo Scientific) containing 2 mM L-glutamine, and 2% (v/v) B27 (Trademark) Supplement (Thermo Scientific). At 4 days in vitro, half of the culture medium was replaced by freshly prepared maintenance medium and replaced similarly every 3 or 4 days.

### HEK293T Cells:

Human embryonic kidney (HEK) 293T cells (Lenti-X (Trademark) 293T Cell Line, Clontech) were seeded at 25% confluence on 100 mm BioCoat (Trademark) Collagen I pre-coated culture dishes (Corning).and cultured in DMEM high glucose (Thermo Scientific) containing 1 mM sodium pyruvate (Thermo Scientific) and 10% (v/v) fetal bovine serum (Biological Industries) at 37° C. under 5% CO₂. Cells were passaged at 90% confluence by trypsinization and reseeded.

### Lentivirus Preparation and Titration:

When reaching 90% confluence, HEK293T cell dishes were each transfected with 7.3 μg of pLVTHM (transfer plasmid containing the shRNA of interest and a GFP reporter gene (8), 5 μg of psPAX2, and 2.3 μg of pMD2.G (lentivirus packaging plasmids, gifts from Didier Trono, Addgene plasmids #12247, #12260, and #12259 respectively), using 75 μg of Polyethylenimine Max 40K (Polysciences Inc.) in 1 mL Opti-MEM (Trademark) (Thermo Scientific) added to the culture dish. Cells were incubated for 7 hrs at 37° C. under 5% CO₂, then media were replaced with 8 mL of fresh culture media. Cell culture supernatants (containing lentiviruses) were collected after 48 hrs, and filtered through 0.45 μm syringe filter, before ultracentrifugation for 2 hrs 87,000 g at 4° C. (JS-24.15 rotor, Beckman Coulter). Lentiviral pellets were then resuspended with PBS, placed on ice for 2 hrs, aliquoted (3×5 μL of lentiviral suspensions from each HEK293T cell culture dish), and stored at −80° C.

To titrate the pLVTHM-based lentiviruses, primary hippocampal cultures at days in vitro (DIV) 6 were used and infected with serial dilutions (1:1,000 to 1:500,000) in fresh maintenance medium of a frozen lentiviral aliquot. After 24 hrs, the medium containing lentiviruses was replaced by fresh maintenance medium. Infected cultures dishes were then observed at DIV11-12 with a confocal microscope (LSM 780, Zeiss). Images were acquired and analyzed with brightness and contrast set at the limit of appearance of autofluorescence on the control uninfected dish. The total number of neurons and GFP positive neurons were counted to calculate the biological titer (BT) in transducing units (TU) per mL according to the formula described in (9): BT=(F×N×D)/V; where F is the percentage of GFP positive cells, N is the number of cells counted in the dish, D is the dilution factor, and V is the volume in mL of dilution. All lentiviruses used in the experiments presented a biological titer above 3×10⁹ TU/mL at dilution 1:1,000 with a GFP positive infection rate >95%.

shRNA Cloning and Knockdown Assay:

RNA interference knockdown was performed by plasmid-based short hairpin RNA (shRNA) using the sequence 5′-CAGTCAACCTCTTCAGTCA-3′ (SEQ ID NO: 37), which targets efficiently mouse Aak1 at nucleotide position 1808-1826 (Exon 11, previously used and described in (10). The sequence 5′-ACCCTATTCCTGTACTAATTA-3′ (SEQ ID NO: 38) targeting a region in exon 20 of Aak1 and which did not show any interference effect on Aak1 expression in our western blot experiments was used as a negative control (shRNA-control). The specific forward and reverse shRNA oligonucleotides, flanked by restriction sites Mlu1 and Cla1, were designed to contain the sense strand of 19 or 21 nucleotide target sequence, followed by a short spacer (TTCAAGAGA) (SEQ ID NO: 39), and the reverse complement of the sense strand. Five thymidines were added at the end of the oligonucleotide as RNA transcriptional stop signal (see table for full custom sequences). Customized oligos (Fasmac) were annealed at 2 M in annealing buffer (OriGene) at 95° C. for 5 min, followed by incubation at 70° C. for 10 min and slow cooling to room temperature. Annealed oligos were then inserted into pLVTHM lentiviral vector digested by Mlu1 and Cla1, downstream to the H1 promoter. All constructs were confirmed by sequencing service from Fasmac. Hippocampal neuron cultures at DIV11-12 were infected with GFP and shRNA-expressing lentiviruses (see ‘Lentivirus preparation and titration’). At DIV15, neuronal cells were scraped, proteins extracted, and the expressions of GFP (lentiviral infection reporter) and aak1 (RNAi target) were monitored by western blot (see ‘Western blot characterization’).

### Proteomic Data Repository

The proteomic raw data files of this study are available through the Japan Proteome Standard Repository Database (11). The accession numbers are PXD021549 for ProteomeXchange and JPST000968 for jPOST.

The quality of all P2′ and SV purification procedures was controlled by western blots of synaptic protein markers and by electron microscopy (see ‘Western blotting characterization’ and ‘Electron microscopy imaging’).

### Proteomics Sample Preparation and Mass Spectrometry

### Sequential Protein Digestion

Fifty micrograms of proteins extracted from P2′ or SV (in a volume <30 μl) were resuspended into 200 μl of buffer containing 8 M urea, 100 mM Tris-HCl (pH=8) (‘urea buffer’) and placed onto a Pall Nanosep (Registered trademark) 10K Omega filter (Sigma). After shaking 1 min 850 rpm at room temperature (Eppendorf Thermomixer), samples were centrifuged during 13 min at 6,400×g (conditions that were optimal to remove all the liquid from the upper chamber using a TOMY Kintaro KT-24 centrifuge). After repeating these steps two times, proteins were resuspended with 200 μl of urea buffer containing 50 mM iodoacetamide and incubated in darkness for 1 hr at room temperature. The alkylation was then stopped by centrifuging as above and by resuspending protein samples with 200 μl of urea buffer containing 25 mM DTT. Unfolded proteins were subsequently washed and centrifuged as above three times with 20 mM ammonium bicarbonate. Proteolytic enzymes were used in a ratio of 1:50 with proteins.

In order to generate as many unique peptides as possible, a first digestion step (‘trimming’) was performed using endoproteinase lys-C(Promega) for 6 hrs at 37° C., followed by a second digestion step overnight (16-18 hrs) at 37° C. using a trypsin/lys-C combination (Promega). After centrifugation as above, digested peptides were acidified with 1% TFA, concentrated and dried using a EZ-2 Elite evaporator (SP Scientific).

### Orthogonal Peptide Separations

In order to separate as many generated peptides as possible, an electrostatic repulsion-hydrophilic interaction chromatography or ERLIC-based separation was performed (Alpert et al Anal. Chem. 2010). The following conditions were adapted and optimized to obtain the highest number of identified proteins from P2′ and SV samples. Mobile phase solvents preparation: solvents were freshly prepared for each experiment using Optima® LC/MS Grade acetonitrile (ACN), formic acid (FA) and water from Fisher Chemicals. Solvent A: 90% ACN, 0.1% FA. Ammonium hydroxide (NH₄OH, 25% w/w in water, Fluka) was then added to adjust pH at 4.5. Solvent B: 30% ACN, 0.1% FA. The digested peptide mixture was fractionated on a weak anion exchange PolyWax LP™ column (PolyLC Inc.; 1 mm inner diameter×150 mm, 5 mm particle size, 300 A pore size) using a PAL HTC autosampler (CTC Analytics) for automatic injection and fractions collection, in a gradient mode (3 min solvent A, to 10% B in 7 min, 10% B to 25% B in 24 min, 25% B to 70% B in 16 min, 70% B to 81% B in 6 min, 81% B to 100% B in 3 min, with final wash at 100% B for 6 min and re-equilibration at 100% A for 20 min) at a flow rate of 40 μL/min. Twenty four fractions were collected every 3 min between 0 and 72 min, and subsequently concentrated and dried using a EZ-2 Elite evaporator (SP Scientific).

### Mass Spectrometry Detection (Includes the RPC LC-MS C18-Based Second Separation)

Peptide samples were resuspended in 30 μl of 0.1% formic acid and analyzed using a Q-Exactive Plus Orbitrap hybrid mass spectrometer (Thermo Scientific) equipped with Ultimate 3000 nano-HPLC system (Dionex), HTC-PAL autosampler (CTC Analytics), and nanoelectrospray ion source. Five microliters of each sample were injected into a Zorbax 300SB C18 capillary column (0.3×150 mm, Agilent Technologies) and heated at 40° C. A one-hour HPLC gradient was employed (1% B to 32% B in 45 min, 32% B to 45% B in 15 min, with final wash at 75% B for 5 min and re-equilibration at 1% B for 10 min.) using 0.1% formic acid in distilled water as solvent A, and 0.1% formic acid in acetonitrile as solvent B. A flow rate of 3.5 μL/min was used for peptide separation. Temperature of the heated capillary was 300° C., and 1.9 kV spray voltage was applied to all samples. The mass spectrometer settings were as follow: full MS scan range 350 to 1500 m/z with a mass resolution of 70,000, 30 μs scan time, and automatic gain control set to 1×E6 ions, and fragmentation MS2 of the 20 most intense ions.

### Protein Identification

Protein identification was done using Proteome Discoverer Software v2.1 (Thermo Scientific), and Mascot 2.6 (Matrix Science) as a search engine. A database downloaded from UniprotKB Rattus norvegicus (Proteome ID: UP000002494) was used with search parameters as follow: trypsin enzyme, up to two miscleavages, with precursor and fragment mass tolerance set to 10 ppm and 0.02 Da respectively. Cysteine carbamidomethylation, methionine oxidation, asparagine and glutamine deamidation, and N-terminal protein acetylation were set as variable modifications. The results were filtered using a false discovery rate of <1% as a cutoff threshold, determined by the Percolator algorithm in Proteome Discoverer software.

### Analysis of Proteomics Data (Excel Tables and Volcano Plot)

Results obtained from Proteome Discoverer, peak area scores and peptide spectrum match counting (PSMs), were used for statistical analysis using R software version 3.2.5 (R Project for Statistical Computing). Quasi-Poisson generalized linear models were generated (y~1, y~ treat) and compared using analysis of deviance for generalized linear model fits (Anova) to obtain p-value, using an F-test, and adjusted with Benhamin-Hochberg method.

### DISCUSSION

In this study, we have used SVs purified from rodent brain, as a model for identifying and quantifying the ‘deep proteome’ applying a newly established proteomic workflow. SVs isolated from mammalian brain are morphologically homogeneous (34) and share a set of common proteins, with more than 90% containing the major SV protein synaptophysin (2). Yet, they are heterogeneous with respect to synapse types and neurotransmitter content. With the new proteomic workflow introduced here, we identified ~1,500 proteins in SVs, more than three times as many as previously reported (2, 4, 7). Of these, we found 134 SV-resident proteins, of which 86 are of low abundance (<1 copy per SV). These proteins may therefore be restricted to SV subsets, deduced from the findings that they include previously missed vesicular transporters for monoamines and acetylcholine, present in only a small percentage of brain synapses. Of the ~1,500 SV-fraction proteins, more than 200 have genetic associations with CNS diseases, highlighting the importance of this deep diverse and previously hidden proteome for proper brain functions. A resource database was constructed to include all data on identification, quantitative distribution, and structural and functional annotations for each protein detected in the SV fraction.

The increased peptide coverage of the “UD workflow” is based on two major improvements in combination: (i) enhanced cleavage using proteases in sequence and (ii) the introduction of an off-line orthogonal peptide separation prior to reversed phase LC-MS/MS. These steps resulted in a remarkable increase in unique peptide detection and have greatly expanded the protein inventory of SVs, including highly homologous proteins within families. For example, 40 Rab proteins, having high sequence homology (75-95%), but distinct trafficking functions (20), were identified. Likewise, functionally characterized, but hidden synaptotagmins such as Syt7 (8-10) were detected, together with other family members of unknown functions. Moreover, the high peptide yield of UD proteomics allows unprecedented label-free and highly reliable quantification of most proteins in the dataset. For the first time, we were able to evaluate the copy numbers of many hundreds of proteins, thereby providing a quantitative scope of the whole SV proteome organization. When compared to previous quantitative studies (2, 6), the results largely confirm copy numbers on average per vesicle, except for three proteins, SNAP29, vti1a, and CIC3, that have abundance scores too low to be further considered as major SV proteins. On the other hand, most detected proteins had copy numbers less than 1 per SV on average, revealing much greater SV heterogeneity than previously envisaged.

Our label-free quantification also allowed a quantitative comparison of the proteomes of isolated nerve terminals and purified synaptic vesicles. This was not only the foundation for identifying bona-fide SV residents, but also for distinguishing for the first time between SV resident and potential SV visitor proteins. Remarkably, about 50% of the SV-residents are nontransmembrane proteins, highlighting the high degree of proteome organization, despite molecular crowding at the synapse (6). For example, UD proteomics revealed that among non-transmembrane proteins, Aak1 is a major SV-resident protein, having SV/P2′ ratio of ~4 and a copy number/SV of 1.5. Our functional assays indicated that this kinase is essential to maintain high-frequency neurotransmission by accelerating SV recycling. Thus, our new classification of SV protein repertoires may facilitate functional studies and may result in the identification of major regulators of synaptic transmission.

It needs to be borne in mind that SVs, starting from enriched synaptosomes, are isolated solely based on their size and density. Therefore, heterogeneity may also be caused, at least in part, by the presence of membranes derived from different trafficking steps, such as partially clathrin-uncoated vesicles, small endosomal vesicles, or SVs from axonal compartments en route to nerve terminals. While these compartments are part of the same recycling pathway and are expected to share vesicular membrane-resident proteins, the “visitor” proteins are likely to be different. This may explain the presence of endosomal-related proteins (e.g. Stx7, AP3) or proteins of the active zone (e.g. Piccolo, Bassoon) in the SV proteome. Moreover, we could not exclude the possibility that the SV preparation is contaminated, even to a small extent, with vesicles from other sources, for instance, small vesicles artificially generated from larger membranes during homogenization, or vesicles from postsynaptic side. Indeed, analysis of the UD-SV proteome using the SynGO resource (15) has revealed a postsynaptic contamination of at least 4% based on 691proteins that were annotated in SynGO. Of the presence of contaminants in remaining 775 SV proteins in our study, we cannot make a definitive calculation as these are not annotated in the SynGO database.

A closer look at the defined SV-resident repertoire (proteins with SV/P2′ iBAQ ratios >2) provides important leads towards a better understanding of SV molecular and functional heterogeneity. Of 134 SV-resident proteins, 86 have copy numbers <1/SV. The 40 most abundant SV-resident proteins (in ranks 1-180) include all the subunits of V-ATPase, vesicular “tetraspanins” including SCAMPs, synaptophysins and synaptogyrins, Syts and SV2 proteins, as well as membrane-associated synapsins and CSPs. VGLUT1/2 and VGAT, vesicular transporters of the two major neurotransmitters in the brain, glutamate (excitatory synapses) and GABA (inhibitory synapses), are also in this list. All these proteins are likely present on SVs throughout the entire nervous system.

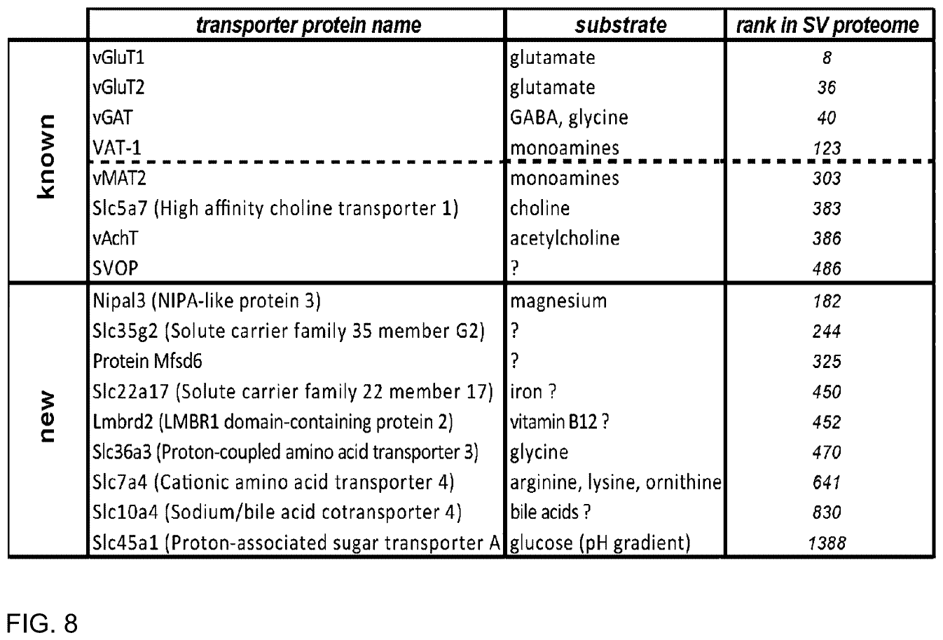

Minor SV-residents (<1 copy per SV, beyond rank 180) include proteins generally involved in membrane trafficking, such as additional SNAREs, Rab GTPases, phospholipid kinases, tethering complexes, and autophagy-related proteins. Their low abundance suggests that they reside on a subset of vesicles within synapses. For example, the copy number of the transmembrane protein, Atg9a, was 1 per 25 SVs (See FIG. 7) implying that 4% of vesicles in the synaptic compartment may be recruited to an autophagic pool. As another possibility, these proteins may be expressed specifically in a small subset of synapses in specific brain regions. Indeed, this list includes the known scarce neurotransmitter vesicular transporters VMAT2, VAChT, Slc5a7 and VGLUT3, reflecting the functional heterogeneity of synapses (FIG. 3C, FIG. 8). Interestingly, our list also includes almost a dozen novel transporter proteins.

Many SV proteins, whether classified as residents or potential visitors, may have specific functions in regulating or maintaining the performance of synapses. In fact, our UD proteomics has detected over 200 proteins in the SV fraction known to be genetically associated with neurological (mental, motor and sensory processing) disorders. Remarkably, a majority of these proteins (76%) was found in low-abundance ranges and had copy numbers <0.04/SV. These neurological disorders likely originate from various synaptic dysfunctions specific to discrete neuronal populations of the nervous system. In fact, recent evidence supports the idea of ‘synaptopathies’ as a causal polygenic mechanism for psychiatric diseases (35-38). In the process of evolution, abundant canonical proteins are often ancestral components, whereas proteins of low abundance tend to emerge for new functions (39). In this respect, the deep diversified synaptic proteome may account for mammalian- or human-specific neurological diseases. This could be a key reason why, despite technical difficulties, investigations of deep subcellular proteomes beyond ‘average models’ are necessary.

### REFERENCES

1. C. Biesemann et al., Proteomic screening of glutamatergic mouse brain synaptosomes isolated by fluorescence activated sorting. EMBO J 33, 157-170 (2014).
2. S. Takamori et al., Molecular anatomy of a trafficking organelle. Cell 127, 831-846 (2006).
3. A. Bayes et al., Comparative study of human and mouse postsynaptic proteomes finds high compositional conservation and abundance differences for key synaptic proteins. PLoS One 7, e46683 (2012).
4. J. Boyken et al., Molecular profiling of synaptic vesicle docking sites reveals novel proteins but few differences between glutamatergic and GABAergic synapses. Neuron 78, 285-297 (2013).
5. N. A. O'Rourke, N. C. Weiler, K. D. Micheva, S. J. Smith, Deep molecular diversity of mammalian synapses: why it matters and how to measure it. Nat Rev Neurosci 13, 365-379 (2012).
6. B. G. Wilhelm et al., Composition of isolated synaptic boutons reveals the amounts of vesicle trafficking proteins. Science 344, 1023-1028 (2014).
7. M. Gronborg et al., Quantitative comparison of glutamatergic and GABAergic synaptic vesicles unveils selectivity for few proteins including MAL2, a novel synaptic vesicle protein. J Neurosci 30, 2-12 (2010).
8. Y. C. Li, N. L. Chanaday, W. Xu, E. T. Kavalali, Synaptotagmin-1- and Synaptotagmin-7-Dependent Fusion Mechanisms Target Synaptic Vesicles to Kinetically Distinct Endocytic Pathways. Neuron 93, 616-631 e613 (2017).
9. S. L. Jackman, J. Turecek, J. E. Belinsky, W. G. Regehr, The calcium sensor synaptotagmin 7 is required for synaptic facilitation. Nature 529, 88-91 (2016).
10. H. Liu et al., Synaptotagmin 7 functions as a Ca²⁺-sensor for synaptic vesicle replenishment. Elife 3, e01524 (2014).
11. R. Aebersold, M. Mann, Mass-spectrometric exploration of proteome structure and function. Nature 537, 347-355 (2016).
12. P. G. Righetti, E. Boschetti, Sherlock Holmes and the proteome—a detective story. FEBS J 274, 897-905 (2007).
13. A. J. Alpert, Electrostatic repulsion hydrophilic interaction chromatography for isocratic separation of charged solutes and selective isolation of phosphopeptides. Anal Chem 80, 62-76 (2008).
14. A. J. Alpert et al., Peptide orientation affects selectivity in ion-exchange chromatography. Anal Chem 82, 5253-5259 (2010).
15. F. Koopmans et al., SynGO: An Evidence-Based, Expert-Curated Knowledge Base for the Synapse. Neuron 103, 217-234 e214 (2019).
16. R. Jahn, T. C. Sudhof, Synaptic vesicles and exocytosis. Annu Rev Neurosci 17, 219-246 (1994).
17. M. Morciano et al., Immunoisolation of two synaptic vesicle pools from synaptosomes: a proteomics analysis. J Neurochem 95, 1732-1745 (2005).
18. N. Hirokawa, K. Sobue, K. Kanda, A. Harada, H. Yorifuji, The cytoskeletal architecture of the presynaptic terminal and molecular structure of synapsin 1. J Cell Biol 108, 111-126 (1989).
19. R. Jahn, D. Fasshauer, Molecular machines governing exocytosis of synaptic vesicles. Nature 490, 201-207 (2012).
20. H. Stenmark, Rab GTPases as coordinators of vesicle traffic. Nat Rev Mol Cell Biol 10, 513-525 (2009).
21. N. J. Pavlos et al., Quantitative analysis of synaptic vesicle Rabs uncovers distinct yet overlapping roles for Rab3a and Rab27b in Ca²⁺-triggered exocytosis. J Neurosci 30, 13441-13453 (2010).
22. N. J. Grimsey, L. J. Coronel, I. C. Cordova, J. Trejo, Recycling and Endosomal Sorting of Protease-activated Receptor-1 Is Distinctly Regulated by Rab11A and Rab11B Proteins. J Biol Chem 291, 2223-2236 (2016).
23. M. Forgac, Vacuolar ATPases: rotary proton pumps in physiology and pathophysiology. Nat Rev Mol Cell Biol 8, 917-929 (2007).
24. M. Merkulova et al., Mapping the H (+) (V)-ATPase interactome: identification of proteins involved in trafficking, folding, assembly and phosphorylation. Sci Rep 5, 14827 (2015).
25. A. Cesar-Razquin et al., A Call for Systematic Research on Solute Carriers. Cell 162, 478-487 (2015).
26. S. Takamori, J. S. Rhee, C. Rosenmund, R. Jahn, Identification of a vesicular glutamate transporter that defines a glutamatergic phenotype in neurons. Nature 407, 189-194 (2000).
27. M. J. Nirenberg, Y. Liu, D. Peter, R. H. Edwards, V. M. Pickel, The vesicular monoamine transporter 2 is present in small synaptic vesicles and preferentially localizes to large dense core vesicles in rat solitary tract nuclei. Proc Natl Acad Sci USA 92, 8773-8777 (1995).
28. S. M. Ferguson et al., Vesicular localization and activity-dependent trafficking of presynaptic choline transporters. J Neurosci 23, 9697-9709 (2003).
29. E. Weihe, J. H. Tao-Cheng, M. K. Schafer, J. D. Erickson, L. E. Eiden, Visualization of the vesicular acetylcholine transporter in cholinergic nerve terminals and its targeting to a specific population of small synaptic vesicles. Proc Natl Acad Sci USA 93, 3547-3552 (1996).
30. R. Janz, K. Hofmann, T. C. Sudhof, SVOP, an evolutionarily conserved synaptic vesicle protein, suggests novel transport functions of synaptic vesicles. J Neurosci 18, 9269-9281 (1998).
31. M. Larhammar et al., SLC10A4 is a vesicular amine-associated transporter modulating dopamine homeostasis. Biol Psychiatry 77, 526-536 (2015).
32. W. Kostich et al., Inhibition of AAK1 Kinase as a Novel Therapeutic Approach to Treat Neuropathic Pain. J Pharmacol Exp Ther 358, 371-386 (2016).
33. L. Y. Wang, L. K. Kaczmarek, High-frequency firing helps replenish the readily releasable pool of synaptic vesicles. Nature 394, 384-388 (1998).
34. W. B. Huttner, W. Schiebler, P. Greengard, P. De Camilli, Synapsin I (protein I), a nerve terminal-specific phosphoprotein. III. Its association with synaptic vesicles studied in a highly purified synaptic vesicle preparation. J Cell Biol 96, 1374-1388 (1983).
35. M. Fromer et al., De novo mutations in schizophrenia implicate synaptic networks. Nature 506, 179-184 (2014).
36. R. Reig-Viader, C. Sindreu, A. Bayes, Synaptic proteomics as a means to identify the molecular basis of mental illness: Are we getting there? Prog Neuropsychopharmacol Biol Psychiatry 84, 353-361 (2018).
37. P. F. Sullivan, M. J. Daly, M. O'Donovan, Genetic architectures of psychiatric disorders: the emerging picture and its implications. Nat Rev Genet 13, 537-551 (2012).
38. A. P. Wingo et al., Large-scale proteomic analysis of human brain identifies proteins associated with cognitive trajectory in advanced age. Nat Commun 10, 1619 (2019).
39. R. D. Emes et al., Evolutionary expansion and anatomical specialization of synapse proteome complexity. Nat Neurosci 11, 799-806 (2008).

1.-22. (canceled)

23. A method for modulating a function of a cell, comprising
contacting the cell with brain-derived neurotrophic factor (BDNF), ciliary neurotrophic factor (CNTF), and glial cell derived neurotrophic factor (GDNF).

24. The method of claim 23, further comprising
contacting the cell with fibroblast growth factor 16 (FGF16) or fibroblast growth factor 22 (FGF22); or fibroblast growth factor 16 (FGF16) and fibroblast growth factor 22 (FGF22).

25. The method of claim 23, further comprising
contacting the cell with one or more ligand(s) selected from the group consisting of FGF3, FGF7, FGF10, FGF22, FGF8, FGF9, FGF16, FGF17, FGF18, FGF20, NRTN, and PSPN.

26. The method of claim 23, further comprising
contacting the cell with NRTN.

27. The method of claim 23, wherein the cell is a stem cell or a stem cell-derived neuronal cell.

28. The method of claim 23, wherein the modulating the function of the stem cell-derived cell is selected from the group consisting of enhancing synaptogenesis, improving neuronal morphogenesis, improving neuronal growth, and improving neuronal activity.

29. The method of claim 26, wherein the stem cell is an induced pluripotent stem cell (iPSC) and/or an embryonic stem cell (ESC).

30. A method for treating a disease and/or a disorder associated with a nervous system in a subject in need thereof, comprising:
obtaining a cell from the subject;
modulating the function of the cell by the method of claim 23; and
administering the cell to the subject.

31. The method of claim 30, wherein the disease and/or the disorder associated with a nervous system is selected from the group consisting of Alzheimer's disease, dementia, schizophrenia, autism, ADHD, Parkinson's, multiple sclerosis, epilepsy, Charcot-Marie-Tooth disease, Retinitis, metabolic neurodegenerative disease, dystonia, intellectual disability, deafness, spinocerebellar ataxia, sleep disorders, cortical dysplasia, dyslexia, microphthalmia, leukodystrophy, and dyskinesia.

32. A kit comprising brain-derived neurotrophic factor (BDNF), ciliary neurotrophic factor (CNTF) and glial cell-derived neurotrophic factor (GDNF); or one or more vectors comprising nucleic acid sequences encoding BDNF, CNTF and GDNF.

33. The kit of claim 32, further comprising fibroblast growth factor 16 (FGF16) or fibroblast growth factor 22 (FGF22); or fibroblast growth factor 16 (FGF16) and fibroblast growth factor 22 (FGF22); or wherein the one or more vectors comprise nucleic acid sequences encoding FGF16 or FGF22; or nucleic acid sequences encoding FGF16 and FGF22.

34. The kit of claim 32, further comprising one or more of FGF3, FGF7, FGF10, FGF22, FGF8, FGF9, FGF16, FGF17, FGF18, FGF20, NRTN and PSPN; or wherein the one or more vectors comprise nucleic acid sequences encoding one or more of FGF3, FGF7, FGF10, FGF22, FGF8, FGF9, FGF16, FGF17, FGF18, FGF20, NRTN, and PSPN.

35. The kit of claim 32, further comprising NRTN; or wherein the one or more vectors further comprises a nucleic acid sequence encoding NRTN.

36. The kit of claim 32, further comprising containers.

37. The kit of claim 32, further comprising a package insert containing instructions for use of the kit.


SEQUENCE LISTING (39 sequences)

SEQ ID NO: 1 - protein, 8 aa, Rattus norvegicus
NILTEIYR

SEQ ID NO: 2 - protein, 29 aa, Rattus norvegicus
NNLSFIETSALDSTNVEEAFKNILTEIYR

SEQ ID NO: 3 - protein, 29 aa, Rattus norvegicus
NGLSFIETSALDSTNVEAAFQTILTEIYR

SEQ ID NO: 4 - protein, 8 aa, Rattus norvegicus
AITSAYYR

SEQ ID NO: 5 - protein, 7 aa, Rattus norvegicus
SIQVDGK

SEQ ID NO: 6 - protein, 11 aa, Rattus norvegicus
VVLIGDSGVGK

SEQ ID NO: 7 - protein, 7 aa, Rattus norvegicus
SIQVDGK

SEQ ID NO: 8 - protein, 13 aa, Rattus norvegicus
GAVGALLVYDIAK

SEQ ID NO: 9 - protein, 11 aa, Rattus norvegicus
FTRNEFNLESK

SEQ ID NO: 10 - protein, 10 aa, Rattus norvegicus
STIGVEFATR

SEQ ID NO: 11 - protein, 9 aa, Rattus norvegicus
HLTYENVER

SEQ ID NO: 12 - protein, 6 aa, Rattus norvegicus
SNLLSR

SEQ ID NO: 13 - protein, 11 aa, Rattus norvegicus
AQIWDTAGQER

SEQ ID NO: 14 - protein, 15 aa, Rattus norvegicus
DHADSNIVIMLVGNK

SEQ ID NO: 15 - protein, 8 aa, Rattus norvegicus
NEFNLESK

SEQ ID NO: 16 - protein, 12 aa, Rattus norvegicus
GTRDDEYDYLFK

SEQ ID NO: 17 - protein, 8 aa, Rattus norvegicus
AVPTDEAR

SEQ ID NO: 18 - protein, 580 aa, Rattus norvegicus
MESQCDYSMYFPAVPLPPRAELAGDPGRYRALPRRNHLYLGETVRFLLVLRCRGSAGPGVGGGTGLASRGAWTELATALAALASVSAGGVLPGCGSAGDQDADPPGGGDPGGGGLFRSCSPLLTHGPGPATSGGATTLPMEEPIVSTDEVIFPLTVSLDRLPPGTPKAKIVVTVWKREVEAPEVRDQGYLRLLQTRSPGETFRGEQSAFKAQVSTLLTLLPPPVLKCRQFTVAGKHLTVLKVLNSSSQEEISIWDIRILPNFNASYLPVMPDGSVLLVDNVCHQSGEVSMGSFCRLPGTSGYFPCPLSALEEHNFLFQLRGGEQPPPGAKEGLEVPLIAVVQWSTPKLPFTQSIYTHYRLPSVRLDRPCFVMTASCESPVRTYERFTVTYTLLNNLQDFLAVRLVWTPEHAQAGKQLCEEERRAMQAALDSIVCHTPLNNLGFSRKGSALTFSVAFQALRTGLFELSQHMKLKLQFTASVSHPPPEARPLSRKSSPSSPAVRDLVERHQASLGRSQSFSHQQPSRSHLMRSGSVMERRAITPPVASPVGRPLYLPPDKAVLSLDKIAKRECKVLVVEPVK

SEQ ID NO: 19 - protein, 8 aa, Rattus norvegicus
VLVVEPVK

SEQ ID NO: 20 - protein, 8 aa, Rattus norvegicus
EVEAPEVR

SEQ ID NO: 21 - protein, 12 aa, Rattus norvegicus
LVWTPEHAQAGK

SEQ ID NO: 22 - protein, 11 aa, Rattus norvegicus
RNHLYLGETVR

SEQ ID NO: 23 - protein, 11 aa, Rattus norvegicus
GKEMEIPQVER

SEQ ID NO: 24 - protein, 15 aa, Rattus norvegicus
EALDEDDDTDISYKK

SEQ ID NO: 25 - protein, 7 aa, Rattus norvegicus
YGSFISR

SEQ ID NO: 26 - protein, 12 aa, Rattus norvegicus
QSLTLSSTFTNK

SEQ ID NO: 27 - protein, 12 aa, Rattus norvegicus
TLFGTMDTQPPR

SEQ ID NO: 28 - protein, 9 aa, Rattus norvegicus
GQFQSFAEK

SEQ ID NO: 29 - protein, 8 aa, Rattus norvegicus
STPGVPYR

SEQ ID NO: 30 - protein, 9 aa, Rattus norvegicus
GMTVQPDLK

SEQ ID NO: 31 - protein, 9 aa, Rattus norvegicus
SGGFSITGK

SEQ ID NO: 32 - protein, 7 aa, Rattus norvegicus
LLPEVLR

SEQ ID NO: 33 - protein, 10 aa, Rattus norvegicus
QIEEAQSVLR

SEQ ID NO: 34 - protein, 15 aa, Rattus norvegicus
LKPLEESSTETSLRR

SEQ ID NO: 35 - DNA, 33 bp, synthetic construct
  source 1..33: mol_type = other DNA
tcgacgcgcgcggccaccatggtgagcgagctg

SEQ ID NO: 36 - DNA, 28 bp, synthetic construct
  source 1..28: mol_type = other DNA
tatgactagtatttacctccatcaccag

SEQ ID NO: 37 - RNA, 19 nt, synthetic construct
  source 1..19: mol_type = other RNA
cagtcaacctcttcagtca

SEQ ID NO: 38 - RNA, 21 nt, synthetic construct
  source 1..21: mol_type = other RNA
accctattcctgtactaatta

SEQ ID NO: 39 - skipped